(19) United States
(12) Patent Application Publication
(10) Pub. No.: US 2023/0181482 A1
(43) Pub. Date: Jun. 15, 2023

(54) LIPIDS FOR DELIVERY OF NUCLEIC ACID SEGMENTS

(71) Applicant: AstraZeneca AB, Sodertalje (SE)

(72) Inventors: Martin HEMMERLING, SODERTALJE (SE); Werngard CZECHTIZKY, SODERTALJE (SE); David ULKOSKI, Wilmington, DE (US); Aditya Ravindra POTE, Wilmington, DE (US); Lennart LINDFORS, Sodertalje (SE)

(21) Appl. No.: 18/056,367

(22) Filed: Nov. 17, 2022

Related U.S. Application Data
(60) Provisional application No. 63/264,263, filed on Nov. 18, 2021.
(60) Provisional application No. 63/374,756, filed on Sep. 7, 2022.

(51) Int. Cl. A61K 9/51; A61K 48/00; C07D 305/14; C07D 309/14; C07D 307/14; C07D 309/04; C07D 305/06; C07D 319/12; C07D 311/96; C07D 307/94

(52) U.S. Cl. CPC A61K 9/5123; A61K 9/5146; A61K 48/0033; C07D 305/14; C07D 309/14; C07D 307/14; C07D 309/04; C07D 305/06; C07D 319/12; C07D 311/96; C07D 307/94

(57) ABSTRACT

Disclosed herein are compounds of Formula (I), Formula (III) or Formula (IIIa), or pharmaceutically acceptable salts thereof, wherein A, L, X¹, X², a, b, R¹ and R² are as defined herein. Also disclosed are lipid nanoparticles comprising a compound of Formula (I), Formula (III) or Formula (IIIa), or a pharmaceutically acceptable salt thereof; pharmaceutical compositions comprising a plurality of lipid nanoparticles comprising a compound of Formula (I), Formula (III) or Formula (IIIa), or a pharmaceutically acceptable salt thereof and a nucleic acid segment; as well as methods for delivering a nucleic acid segment comprising administering a plurality of lipid nanoparticles comprising a compound of Formula (I), Formula (III) or Formula (IIIa), or a pharmaceutically acceptable salt thereof, and a nucleic acid segment.

### CROSS-REFERENCE TO RELATED APPLICATIONS

This application claims the benefit of priority to U.S. Provisional Application No. 63/264,263, filed on Nov. 18, 2021; and 63/374,756 filed on Sep. 7, 2022. Each of the above listed applications is incorporated by reference herein in its entirety for all purpose.

### BACKGROUND

Nucleic acid segments such as oligonucleotides (e.g., RNA, for example, messenger RNAs [mRNAs] and small interfering RNAs [siRNAs], antisense oligonucleotides [ASOs] and DNA) have broad potential as new therapeutic treatments for a variety of diseases and disorders. However, challenges remain in administering oligonucleotide therapeutics. Typical formulations include encapsulating the oligonucleotide into a lipid nanoparticle (LNP). LNP formulations usually include (a) an ionizable or cationic lipid or polymeric material bearing a tertiary or quaternary amine to encapsulate the polyanionic mRNA; (b) a zwitterionic lipid that resembles the lipids in the cell membrane; (c) cholesterol to stabilize the lipid bilayer of the LNP; and (d) a polyethylene glycol (PEG)-lipid to give the nanoparticle a hydrating layer, improve colloidal stability and reduce protein absorption. (see Kowalski et al., Molecular Therapy, 27 (4), (2019), 710-728).

In 2018, the FDA approved the first RNA interference therapy, PATISIRAN, for the treatment of polyneuropathy in people with hereditary transthyretin-mediated amyloidosis, which is intravenously delivered using an LNP that incorporates an ionizable lipid (DLin-MC3-DMA, [MC3]). MC3, however, might not be suitable for all delivery systems, depending on the targeted organ, intended delivery route and required therapeutic window. Dose-limiting toxicities were reported from studies in two toxicology-relevant test species, rat and monkey, that were related to MC3-based LNP formulation rather than the delivered cargo. (see Sedic et al., Vet. Pathol. 55 (2), (2018), 341-354). Recently, lipid nanoparticle technology has also successfully been applied to generate the first approved mRNA products for prophylactic vaccination against SARS-COV-2 virus (see e.g. Shoenmaker et al, International Journal of Pharmaceutics, 601, (2021), 120586). However, there remains a need to develop new ionizable lipids for use in lipid nanoparticle formulations for delivery of oligonucleotide therapeutics.

### SUMMARY

In some embodiments, disclosed is a compound of Formula (I):

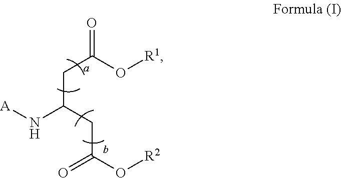

or a pharmaceutically acceptable salt thereof, wherein

A is

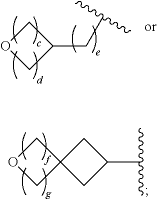

a and b are each independently 6, 7 or 8;

c, d, f and g are each independently 1 or 2;

e is 0, 1 or 2;

R¹ and R² are each independently

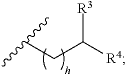

h is 0, 1, 2 or 3;

R³ and R⁴ are each independently —(CH₂)ᵢCH₃; and

i is 3, 4, 5, 6 or 7.

In some embodiments, disclosed is a compound of Formula (III):

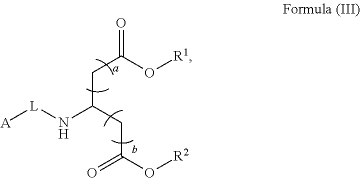

or a pharmaceutically acceptable salt thereof; wherein

A is 4- to 6-membered monocyclic oxacyclyl or 6- to 10-membered bicyclic oxacyclyl;

L is a covalent bond or C1-C3 alkylene;

a and b are each independently 5, 6, 7 or 8; and

R¹ and R² are each independently C₇-C₁₁ straight chain alkyl or C₉-C₁₉ branched alkyl; provided that R¹ and R² is are not both C₇-C₁₁ straight chain alkyl.

In some embodiments, disclosed is a compound of Formula (IIIa):

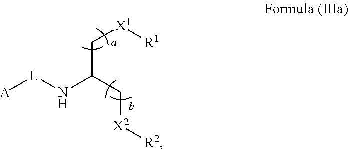

or a pharmaceutically acceptable salt thereof; wherein

A is 4- to 6-membered monocyclic oxacyclyl or 6- to 10-membered bicyclic oxacyclyl;

L is a covalent bond or C₁-C₃ alkylene;

X¹ and X² are each independently

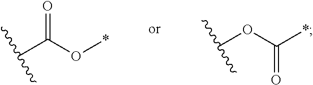

* indicates the attachment point to R¹;

a and b are each independently 4, 5, 6, 7, 8 or 9; provided that when one of a and b is 4 or 5, then the other is 6, 7, 8 or 9;

R¹ and R² are each independently C₇-C₁₁ straight chain alkyl, C₇-C₁₉ branched alkyl, or C₇-C₁₉ alkylene-cyclopropylene-alkyl; provided that R¹ and R² are not both C₇-C₁₁ straight chain alkyl or both C₇-C₁₉ alkylene-cyclopropylene-alkyl.

In some embodiments, disclosed is a lipid nanoparticle comprising a compound of Formula (I), Formula (III) or Formula (IIIa), or a pharmaceutically acceptable salt thereof.

In some embodiments, disclosed is a pharmaceutical composition comprising a plurality of lipid nanoparticles comprising a compound of Formula (I), Formula (III) or Formula (IIIa), or a pharmaceutically acceptable salt thereof; and a nucleic acid segment.

In some embodiments, disclosed is a method of treating a disease or disorder in a subject, comprising administering to the subject a therapeutically effective amount of a pharmaceutical composition as described herein.

In some embodiments, disclosed is a pharmaceutical composition as described herein for use in the treatment of a disease or disorder.

### BRIEF DESCRIPTION OF THE DRAWINGS

FIG. 1 illustrates the expression of eGFP in rat lung homogenate at 24 hours after intratracheal administration of LNP formulations comprising Compound 1 and MC3.

FIG. 2 illustrates the BALF neutrophil concentration in rat BALF at 24 hours after intratracheal administration of LNP formulations comprising Compound 1 and MC3.

FIG. 3 illustrates the expression of eGFP in rat heart at 24 hours after intracardiac administration of LNP formulations comprising Compound 1 and MOD5.

FIG. 4 illustrates the ratio of eGFP expressed in rat liver/rat heart at 24 hours after intracardiac administration of LNP formulations comprising Compound 1 and MOD5.

FIG. 5 illustrates the level of plasma KC in rat at 24 hours after intracardiac administration of LNP formulations comprising Compound 1 and MOD5.

FIG. 6 illustrates the level of plasma IL-6 in rat at 24 hours after intracardiac administration of LNP formulations comprising Compound 1 and MOD5.

FIG. 7 illustrates the level of plasma IP-10 in rat at 24 hours after intracardiac administration of LNP formulations comprising Compound 1 and MOD5.

FIG. 8 illustrates the level of plasma MCP-1 in rat at 24 hours after intracardiac administration of LNP formulations comprising Compound 1 and MOD5.

FIG. 9 illustrates the expression of eGFP in mouse liver at 24 hours after intravenous administration of LNP formulations comprising Compound 1, Compound 2, Compound 3, Compound 4, Compound 5, and MC3.

FIG. 10 illustrates the expression of eGFP in mouse muscle at 24 hours after intramuscular administration of LNP formulations comprising Compound 1, Compound 4 and MOD8.

FIG. 11 illustrates the expression of eGFP in mouse liver at 24 hours after intramuscular administration of LNP formulations comprising Compound 1, Compound 4 and MOD8.

### DETAILED DESCRIPTION

In some embodiments, disclosed is a compound of Formula (I):

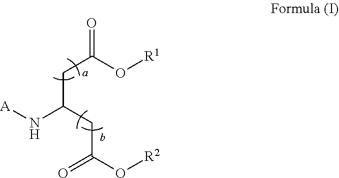

or a pharmaceutically acceptable salt thereof, wherein

### A is

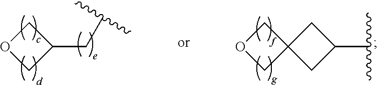

a and b are each independently 6, 7 or 8;
c, d, f and g are each independently 1 or 2;
e is 0, 1 or 2;
R¹ and R² are each independently

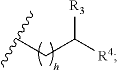

h is 0, 1, 2 or 3;
R³ and R⁴ are each independently —(CH₂)ᵢCH₃; and
i is 3, 4, 5, 6 or 7.

In some embodiments of the compound of Formula (I),

### A is

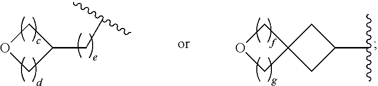

wherein
c, d, f and g are each independently 1 or 2; and
e is 0, 1 or 2.

In some embodiments of the compound of Formula (I), e is 0 or 1.

In some embodiments of the compound of Formula (I), e is 0.

In some embodiments of the compound of Formula (I), e is 1.

In some embodiments of the compound of Formula (I),

A is selected from:

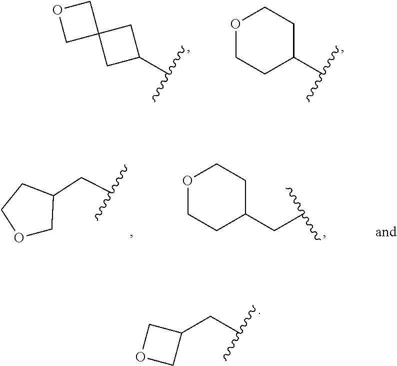 [SMILES: *C1CC2(COC2)C1 ; *C1CCOCC1 ; *CC1CCOC1 ; *CC1CCOCC1 ; *CC1COC1]

In some embodiments of the compound of Formula (I), a and b are each independently 6, 7 or 8;

In some embodiments of the compound of Formula (I), a is 7.

In some embodiments of the compound of Formula (I), b is 7.

In some embodiments of the compound of Formula (I),

R¹ and R² are each independently

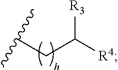

wherein
h is 1 or 2;
R³ and R⁴ are each independently —(CH₂)ᵢCH₃; and
i is 3, 4, 5, 6 or 7.

In some embodiments of the compound of Formula (I), h is 2.

In some embodiments of the compound of Formula (I), i is 3, 4, or 5.

In some embodiments of the compound of Formula (I), i is 4.

In some embodiments of the compound of Formula (I), R³ is —(CH₂)₄CH₃.

In some embodiments of the compound of Formula (I), R⁴ is —(CH₂)₄CH₃.

In some embodiments of the compound of Formula (I), R³ and R⁴ are each —(CH₂)₄CH₃.

In some embodiments, disclosed is a compound of Formula (III):

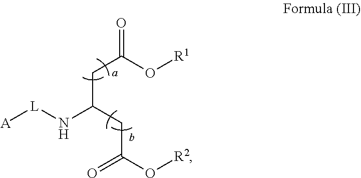

or a pharmaceutically acceptable salt thereof; wherein
A is 4- to 6-membered monocyclic oxacyclyl or 6- to 10-membered bicyclic oxacyclyl;
L is a covalent bond or C₁-C₃ alkylene;
a and b are each independently 5, 6, 7 or 8; and
R¹ and R² are each independently C₇-C₁₁ straight chain alkyl or C₉-C₁₉ branched alkyl; provided that R¹ and R² is are not both C₇-C₁₁ straight chain alkyl.

In one embodiment of the compound of Formula (III), R¹ and R² together contain not more than 31 carbon atoms. In another embodiment of the compound of Formula (III), R¹ and R² together contain not less than 22 carbon atoms. In another embodiment of the compound of Formula (III), R¹ and R² together contain 24 to 30 carbon atoms.

In some embodiments, disclosed is a compound of Formula (IIIa):

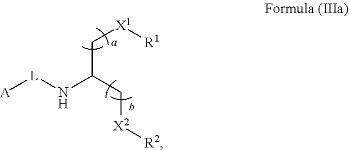

or a pharmaceutically acceptable salt thereof; wherein
A is 4- to 6-membered monocyclic oxacyclyl or 6- to 10-membered bicyclic oxacyclyl;
L is a covalent bond or C₁-C₃ alkylene;
X¹ and X² are each independently

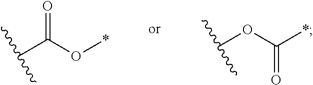

* indicates the attachment point to R¹;
a and b are each independently 4, 5, 6, 7, 8 or 9; provided that when one of a and b is 4 or 5, then the other is 6, 7, 8 or 9; that is, when a is 4 or 5, then b is 6, 7, 8 or 9; and when b is 4 or 5, then a is 6, 7, 8 or 9; and
R¹ and R² are each independently C₇-C₁₁ straight chain alkyl, C₇-C₁₉ branched alkyl, or C₇-C₁₉ alkylene-cyclopropylene-alkyl; provided that (a) R¹ and R² are not both C₇-C₁₁ straight chain alkyl, and (b) R¹ and R² are not both C₇-C₁₉ alkylene-cyclopropylene-alkyl. In some embodiments, R¹ and R² have same number of carbon atoms, wherein R¹ and R² may have the same or different chemical structures. In other embodiments, R¹ and R² have different number of carbon atoms.

In one embodiment of the compound of Formula (IIIa), R¹ and R² together contain not more than 31 carbon atoms. In another embodiment of the compound of Formula (IIIa), R¹ and R² together contain not less than 22 carbon atoms. In another embodiment of the compound of Formula (IIIa), R¹ and R² together contain 24 to 30 carbon atoms.

In some embodiments of the compound of Formula (IIIa), X¹ and X² are both

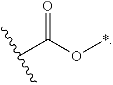

In some embodiments of the compound of Formula (IIIa), X¹ is

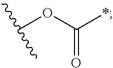

### and X² is

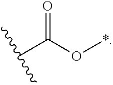

In some embodiments of the compound of Formula (III) or Formula (IIIa),

### A is

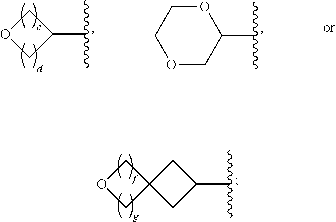

c is 0, 1 or 2;
d is 1, 2 or 3; provided that the sum of c and d is from 2 to 4;
f is 0, 1 or 2; and
g is 1, 2 or 3; provided that the sum of f and g is from 2 to 4.

In some embodiments of the compound of Formula (III) or Formula (IIIa), L is a covalent bond, —CH₂—, or —CH₂CH₂—.

In some embodiments of the compound of Formula (III), R¹ and R² are both C₉-C₁₉ branched alkyl and contain the same number of carbon atoms. In some embodiments, R¹ and R² are two identical C₉-C₁₉ branched alkyl groups.

In some embodiments of the compound of Formula (III), R¹ is C₇-C₁₁ straight chain alkyl or C₉-C₁₉ branched alkyl; R² is C₉-C₁₉ branched alkyl; and provided that R¹ and R² are not identical. In some embodiments, R¹ and R² do not contain the same number of carbon atoms.

In some embodiments of the compound of Formula (IIIa), R¹ is C₇-C₁₁ straight chain alkyl; and R² is C₇-C₁₉ branched alkyl. In some embodiments, R¹ is C₇-C₁₁ straight chain alkyl; and R² is C₁₁-C₁₉ branched alkyl. In another embodiment, R¹ is C₇-C₁₁ straight chain alkyl; and R² is C₁₃-C₁₉ branched alkyl.

In some embodiments of the compound of Formula (IIIa), R¹ is C₇-C₁₉ branched alkyl or C₇-C₁₉ alkylene-cyclopropylene-alkyl; R² is C₇-C₁₉ branched alkyl; and provided that R¹ and R² are not identical. That is, when R¹ and R² are both C₇-C₁₉ branched alkyl, R¹ and R² do not have the same chemical structure regardless of whether R¹ and R² may have the same number of carbon atoms. In some embodiments, R¹ is C₇-C₁₅ branched alkyl or C₇-C₁₅ alkylene-cyclopropylene-alkyl; and R² is C₁₃-C₁₉ branched alkyl. In some embodiment, R¹ is C₇-C₁₃ branched alkyl; and R² is C₁₃-C₁₉ branched alkyl.

In some embodiments of the compound of Formula (IIIa), a and b are the same and are both 6, 7, or 8.

In some embodiments of the compound of Formula (IIIa), a and b are not the same and are each independently 4 to 9, provided that (a) when one of a and b is 4 or 5, then the other is 6, 7, 8 or 9; and (b) the sum of a and b is 12 to 16.

In some embodiments of the compound of Formula (III),

R¹ is —(CH₂)ₘ—CH₃ or

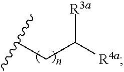

### R² is

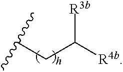

m is 7, 8, or 9;
n and h are each independently 0, 1, 2, or 3;
R^{3a} and R^{4a} are each independently —(CH₂)ₚCH₃;
R^{3b} and R^{4b} are each independently —(CH₂)_{q}CH₃;
p and q are each independently 0, 1, 2, 3, 4, 5, 6, 7, 8, or 9; and provided that R^{3a} and R^{4a}, together with the carbon atom to which they are attached, contain at least 9 carbon atoms and R^{3b} and R^{4b}, together with the carbon atom to which they are attached, contain at least 9 carbon atoms.

In some embodiments of the compound of Formula (III), R¹ and R² together contain no more than 19 carbon atoms.

In some embodiments of the compound of Formula (IIIa),

R¹ is —(CH₂)ₘ—CH₃,

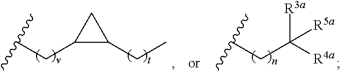

### R² is

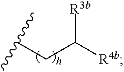

m is 6, 7, 8, or 9;
v is 1, 2 or 3;
t is 3, 4, 5, 6, 7, or 8;
n and h are each independently 0, 1, 2, or 3;
R^{3a} and R^{4a} are each independently —(CH₂)ₚCH₃;
R^{3b} and R^{4b} are each independently —(CH₂)_{q}CH₃;
R^{5a} is hydrogen or methyl;
p and q are each independently 0, 1, 2, 3, 4, 5, 6, 7, 8, or 9; and provided that R^{3a}, R^{4a} and R^{5a}, together with the carbon atom to which they are attached, contain at least 7 carbon atoms and R^{3b} and R^{4b}, together with the carbon atom to which they are attached, contain at least 9 carbon atoms.

In some embodiments of the compound of Formula (IIIa), R^{3a} is —(CH₂)ₚCH₃, wherein p is 0, 1, 2 or 3; and R^{4a} is —(CH₂)_{q}CH₃, wherein p is 4, 5, 6, 7, 8 or 9.

In some embodiments of the compound of Formula (IIIa), R^{3b} and R^{4b} are both —(CH₂)_{q}CH₃, wherein q is 5, 6, 7 or 8.

In some embodiments of the compound of Formula (III), the compound is represented by Formula (IV):

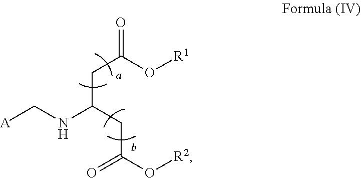

or a pharmaceutically acceptable salt thereof; wherein

### A is

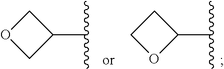

a and b are each independently 5, 6, 7 or 8;
R¹ is C₇-C₁₁ straight chain alkyl or C₉-C₁₉ branched alkyl; and
R² is C₉-C₁₉ branched alkyl.

In some embodiments of the compound of Formula (IV), R¹ and R² are both C₁₀-C₁₇ branched alkyl and contain the same number of carbon atoms.

In some embodiments of the compound of Formula (IV), R¹ is C₇-C₁₁ straight chain alkyl or C₉-C₁₃ branched alkyl; R² is C₁₃-C₁₉ branched alkyl; and provided that R¹ and R² are not both C₁₃ branched alkyl.

In some embodiments of the compound of Formula (IV), a and b are both 5, 6, 7 or 8.

In some embodiments of the compound of Formula (IIIa), the compound is represented by Formula (IVa):

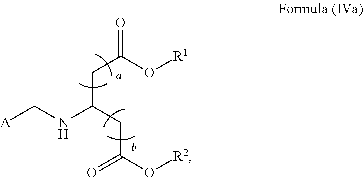

or a pharmaceutically acceptable salt thereof; wherein

### A is

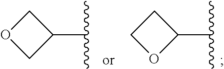

a and b are each independently 5, 6, 7 or 8;
R¹ is C₇-C₁₁ straight chain alkyl, C₇-C₁₅ branched alkyl or C₇-C₁₅ alkylene-cyclopropylene-alkyl; and
R² is C₁₅-C₁₉ branched alkyl.

In some embodiments of Formula (IVa), a and b are same and are both 5, 6, 7 or 8.

In some embodiments of the compound of Formula (III), the compound is represented by Formula (V):

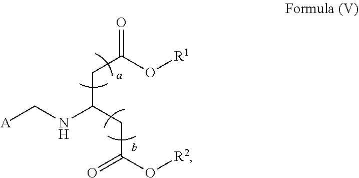

or a pharmaceutically acceptable salt thereof; wherein

### A is

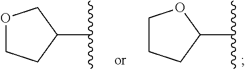 [SMILES: *C1CCOC1 ; *C1CCCO1]

a and b are each independently 5, 6, 7 or 8;
R¹ is C₇-C₁₁ straight chain alkyl or C₉-C₁₉ branched alkyl; and
R² is C₉-C₁₉ branched alkyl.

In some embodiments of the compound of Formula (V), R¹ and R² are both C₁₀-C₁₇ branched alkyl and contain the same number of carbon atoms.

In some embodiments of the compound of Formula (V), R¹ is C₇-C₁₁ straight chain alkyl or C₉-C₁₃ branched alkyl; R² is C₁₃-C₁₉ branched alkyl; and provided that R¹ and R² are not both C₁₃ branched alkyl.

In some embodiments of the compound of Formula (V), a and b are both 5, 6, 7 or 8.

In some embodiments of the compound of Formula (IIIa), the is represented by Formula (Va):

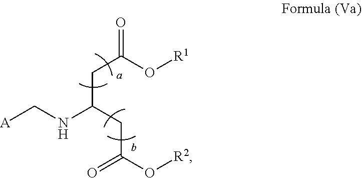

or a pharmaceutically acceptable salt thereof; wherein

### A is

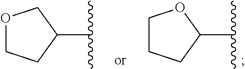 [SMILES: *C1CCOC1 ; *C1CCCO1]

a and b are each independently 5, 6, 7 or 8;
R¹ is C₇-C₁₅ branched alkyl or C₇-C₁₅ alkylene-cyclopropylene-alkyl; and
R² is C₁₅-C₁₉ branched alkyl.

In some embodiments of Formula (Va), a and b are same and are both 5, 6, 7 or 8.

In some embodiments of the compound of Formula (III), the compound is represented by Formula (VI):

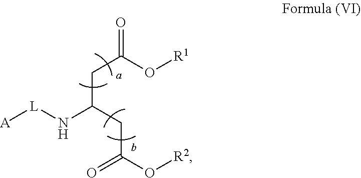

or a pharmaceutically acceptable salt thereof; wherein
A is selected from:

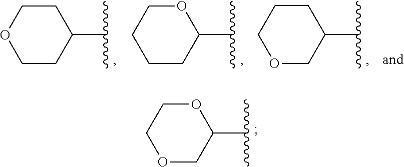

L is a covalent bond, —CH₂—, or —CH₂CH₂—;
a and b are each independently 5, 6, 7 or 8;
R¹ is C₇-C₁₁ straight chain alkyl or C₉-C₁₉ branched alkyl; and
R² is C₉-C₁₉ branched alkyl.

In some embodiments of the compound of Formula (VI), R¹ and R² are both C₁₀-C₁₇ branched alkyl and contain the same number of carbon atoms.

In some embodiments of the compound of Formula (VI), R¹ is C₇-C₁₁ straight chain alkyl or C₉-C₁₃ branched alkyl; R² is C₁₃-C₁₉ branched alkyl; and provided that R¹ and R² are not both C₁₃ branched alkyl.

In some embodiments of the compound of Formula (VI), a and b are both 5, 6, 7 or 8.

In some embodiments of the compound of Formula (IIIa), the compound is represented by Formula (VIa):

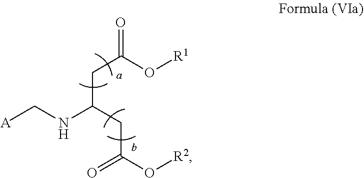

or a pharmaceutically acceptable salt thereof; wherein
A is selected from:

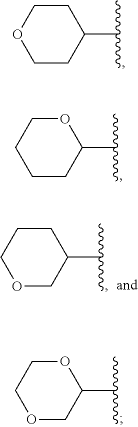 [SMILES: *C1CCOCC1 ; *C1CCCCO1 ; *C1CCCOC1 ; *C1COCCO1]

a and b are each independently 5, 6, 7 or 8;
R¹ is C₇-C₁₅ branched alkyl or C₇-C₁₅ alkylene-cyclopropylene-alkyl; and
R² is C₁₅-C₁₉ branched alkyl.

In some embodiments of Formula (VIa), a and b are same and are both 5, 6, 7 or 8.

In some embodiments of the compound of Formula (III), the compound is represented by Formula (VII):

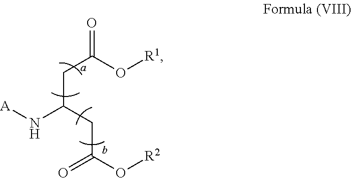

or a pharmaceutically acceptable salt thereof; wherein

### A is

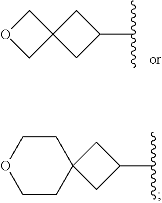 [SMILES: *C1CC2(COC2)C1 ; *C1CC2(CCOCC2)C1]

a and b are each independently 5, 6, 7 or 8;
R¹ is C₇-C₁₁ straight chain alkyl or C₉-C₁₉ branched alkyl; and
R² is C₉-C₁₉ branched alkyl.

In some embodiments of the compound of Formula (VII), R¹ and R² are both C₁₀-C₁₇ branched alkyl and contain the same number of carbon atoms.

In some embodiments of the compound of Formula (VII), R¹ is C₇-C₁₁ straight chain alkyl or C₉-C₁₃ branched alkyl; R² is C₁₃-C₁₉ branched alkyl; and provided that R¹ and R² are not both C₁₃ branched alkyl.

In some embodiments of the compound of Formula (VII), a and b are both 5, 6, 7 or 8.

In some embodiments of the compound of Formula (IIIa), the compound is represented by Formula (VIIa):

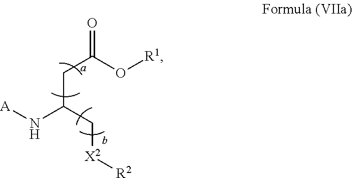

or a pharmaceutically acceptable salt thereof; wherein

### A is

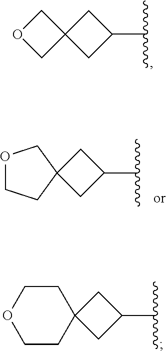 [SMILES: *C1CC2(COC2)C1 ; *C1CC2(CCOC2)C1 ; *C1CC2(CCOCC2)C1]

a and b are each independently 4, 5, 6, 7 or 8; provided that when one of a and b is 4 or 5, then the other is 6, 7, 8 or 9; that is, when a is 4 or 5, then b is 6, 7, 8 or 9; and when b is 4 or 5, then a is 6, 7, 8 or 9;
X¹ and X² are each independently

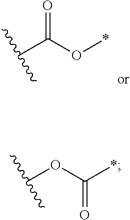

* indicates the attachment point to R¹;
R¹ is C₇-C₁₁ straight chain alkyl or C₇-C₁₅ branched alkyl; and
R² is C₁₅-C₁₉ branched alkyl.

In some embodiments of the compound of Formula (Vila), a and b are both 6, 7, or 8; or alternatively, a and b are each independently 4 to 9; provided that (a) when one of a and b is 4 or 5, then the other is 6, 7, 8 or 9; and (b) the sum of a and b is 12 to 16.

In some embodiments of the compound of Formula (Vila), X¹ and X² are both

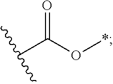

or alternatively, X¹ is

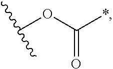

### and X² is

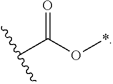

In some embodiments of the compound of Formula (III), Formula (IV), Formula (V), Formula (VI) or Formula (VII), R¹ is selected from

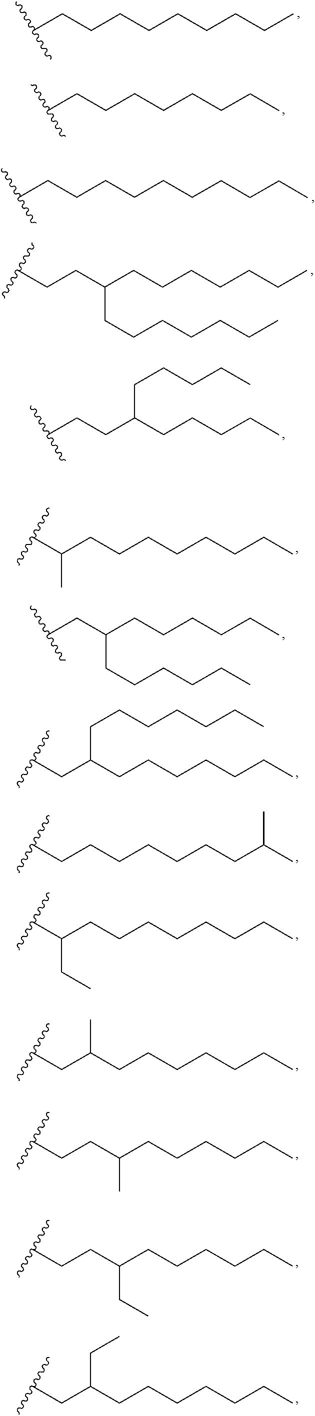

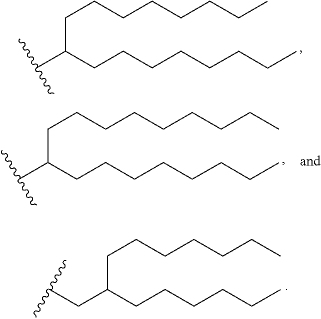 [SMILES: *C(CCCCCCCC)CCCCCCCC ; *C(CCCCCCCC)CCCCCCCCC ; *CC(CCCCCC)CCCCCCC]

In some embodiments of the compound of Formula (IIIa), Formula (IVa), Formula (Va), Formula (VIa) or Formula (VIIa), R¹ is selected from

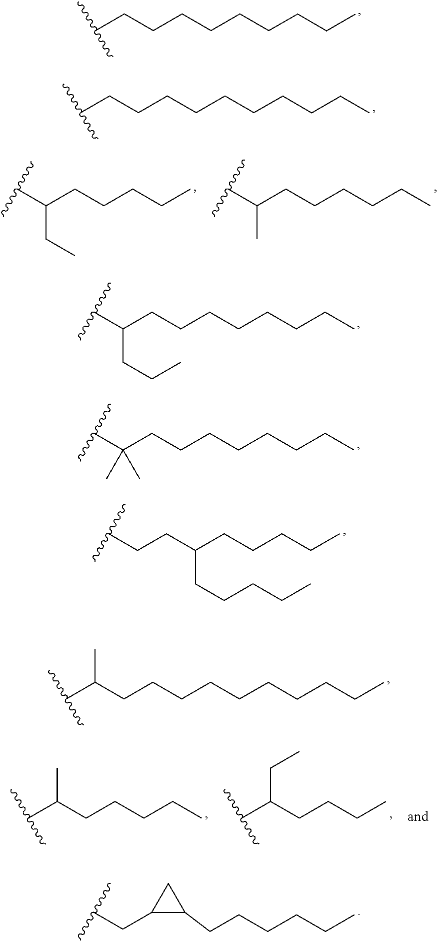

In some embodiments of the compound of Formula (III), Formula (IV), Formula (V), Formula (VI) or Formula (VII), R² is selected from

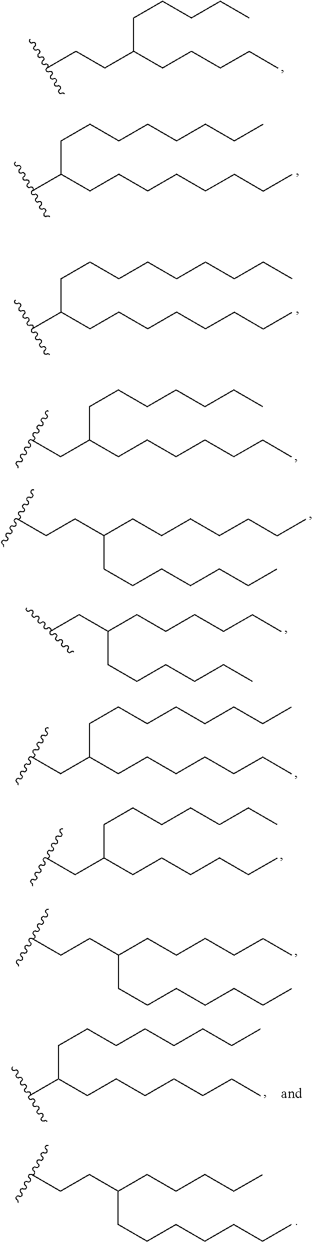

In some embodiments of the compound of Formula (IIIa), Formula (IVa), Formula (Va), Formula (VIa) or Formula (VIIa), R² is selected from

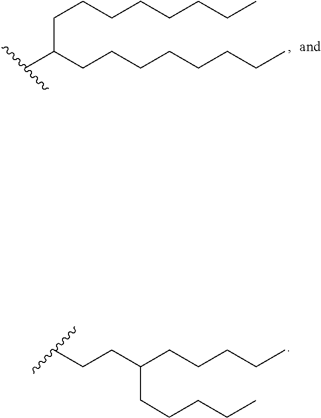 [SMILES: *C(CCCCCCCC)CCCCCCCC ; *CCC(CCCCC)CCCCC]

In some embodiments, the compound of Formula (I) is a compound of Formula (II):

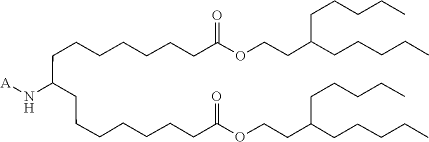

or a pharmaceutically acceptable salt thereof, wherein A is as defined for Formula (I).

In some embodiments, the compound of Formula (I) is bis(3-pentyloctyl) 9-((2-oxaspiro[3.3]heptan-6-yl)amino)heptadecanedioate, or a pharmaceutically acceptable salt thereof.

In some embodiments, the compound of Formula (I) is bis(3-pentyloctyl) 9-((tetrahydro-2H-pyran-4-yl)amino)heptadecanedioate, or a pharmaceutically acceptable salt thereof.

In some embodiments, the compound of Formula (I) bis(3-pentyloctyl) 9-(((tetrahydrofuran-3-yl)methyl)amino)heptadecanedioate, or a pharmaceutically acceptable salt thereof.

In some embodiments, the compound of Formula (I) is bis(3-pentyloctyl) 9-(((tetrahydro-2H-pyran-4-yl)methyl)amino)heptadecanedioate, or a pharmaceutically acceptable salt thereof.

In some embodiments, the compound of Formula (I) is bis(3-pentyloctyl) 9-((oxetan-3-ylmethyl)amino)heptadecanedioate, or a pharmaceutically acceptable salt thereof.

In some embodiments, the compound of Formula (I) is bis(3-pentyloctyl) 9-((2-oxaspiro[3.3]heptan-6-yl)amino)heptadecanedioate.

In some embodiments, the compound of Formula (I) is bis(3-pentyloctyl) 9-((tetrahydro-2H-pyran-4-yl)amino)heptadecanedioate.

In some embodiments, the compound of Formula (I) is bis(3-pentyloctyl) 9-(((tetrahydrofuran-3-yl)methyl)amino)heptadecanedioate.

In some embodiments, the compound of Formula (I) is bis(3-pentyloctyl) 9-(((tetrahydro-2H-pyran-4-yl)methyl)amino)heptadecanedioate.

In some embodiments, the compound of Formula (I) is bis(3-pentyloctyl) 9-((oxetan-3-ylmethyl)amino)heptadecanedioate.

In some embodiments, the compound of Formula (III) or Formula (IIIa) is selected from the exemplified compounds in this disclosure, such as Compounds 6 to 57 as described herein, or a pharmaceutically acceptable salt thereof.

The compounds of formula (I), (II), (111), (IIIa), (IV), (IVa), (V), (Va), (VI), (VIa), (VII) and (VIIa), including any subgenera or species thereof, may have different isomeric forms. The language “optical isomer,” “stereoisomer” or “diastereoisomer” refers to any of the various stereoisomeric configurations which may exist for a given compound of formula (I), (II), (111), (IIIa), (IV), (IVa), (V), (Va), (VI), (VIa), (VII) and (VIIa), including any subgenera or species thereof. It is understood that a substituent may be attached at a chiral center of a carbon atom and, therefore, the disclosed compounds include enantiomers, diastereomers and racemates. The term “enantiomer” includes pairs of stereoisomers that are non-superimposable mirror images of each other. A 1:1 mixture of a pair of enantiomers is a racemic mixture. The term is used to designate a racemic mixture where appropriate. The terms “diastereomers” or “diastereoisomers” include stereoisomers that have at least two asymmetric atoms, but which are not mirror images of each other. The absolute stereochemistry is specified according to the Cahn-Ingold-Prelog R-S system. When a compound is a pure enantiomer, the stereochemistry at each chiral center may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds of formula (I), (II), (III), (IIIa), (IV), (IVa), (V), (Va), (VI), (VIa), (VII) and (VIIa), including any subgenera or species thereof, contain one or more asymmetric centers or axes and may thus give rise to enantiomers, diastereomers or other stereoisomeric forms that may be defined, in terms of absolute stereochemistry, as (R)- or (S)-. The present disclosure is meant to include all such possible isomers, including racemic mixtures, optically pure forms and intermediate mixtures. Optically active (R)- and (S)-isomers may be prepared using chiral synthons or chiral reagents, or resolved using conventional techniques well known in the art, such as chiral HPLC.

As used herein, the term “alkyl” refers to monovalent saturated hydrocarbon radicals having the specified number of carbon atoms. The alkyl includes both straight chain alkyl and branched alkyl. As used herein, the term “alkylene” refers to bivalent saturated hydrocarbon radicals having the specified number of carbon atoms. The alkylene includes both straight chain alkylene and branched alkylene. In this specification the prefix Cx-y, as used in terms such as “Cx-y alkyl” or “Cx-y alkylene” where x and y are integers, indicates the numerical range of carbon atoms that are present in the group. For example, C₇-C₁₁ alkyl refers to an alkyl radical having 7 to 11 carbon atoms. As used herein, the term “cyclopropylene” refers to a bivalent saturated hydrocarbon radical derived from cyclopropane, for example, a chemical moiety having the following structure:

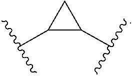 [SMILES: *C1CC1*]

As used herein, the term “oxacyclyl” refers to a heterocyclyl having one or more oxygen atoms in the ring. In one embodiment, the oxacyclyl refers to a ring moiety formed by carbon, oxygen and hydrogen atoms. The oxacyclyl includes both monocyclic and bicyclic oxacyclyl. The bicyclic oxacyclyl includes spiro-bicyclic, wherein the two rings share only one single carbon atom, i.e., the spiro atom; fused or condensed bicyclic, wherein the two rings share two adjacent atoms; and bridged bicyclic, wherein the two rings share three or more atoms, separating the two bridgehead atoms by a bridge containing at least one atom. In one embodiment, the bicyclic oxacyclyl is a spiro-bicyclic oxacyclyl. In this specification the prefix X- to Y-membered, as used in terms such as “X- to Y-membered oxacyclyl” and the like where X and Y are integers, indicates the numerical range of atoms (i.e. carbon atoms and heteroatoms) that are present in the ring and form the ring structure. For example, 4- to 6-membered oxacyclyl refers to an oxacyclyl having 4 to 6 ring atoms.

In some embodiments, disclosed is a compound of Formula (I), Formula (III), or any subgenus or species thereof. In some embodiments, disclosed is a pharmaceutically acceptable salt of the compound of Formula (I), Formula (III), or any subgenus or species thereof. The term “pharmaceutically acceptable salt” includes acid addition salts that retain the biological effectiveness and properties of the compound of Formula (I), Formula (III), or any subgenus or species thereof, and which typically are not biologically or otherwise undesirable. Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethanedisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulfate, naphthoate, napsylate, nicotinate, nitrate, octadecanoate, oleate, oxalate, palmitate, palmoate, phosphate/hydrogen phosphate/dihydrogen phosphate, polygalacturonate, propionate, stearate, succinate, subsalicylate, sulfate/hydrogensulfate, tartrate, tosylate and trifluoroacetate salts. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, trifluoroacetic acid, sulfosalicylic acid, and the like. In some embodiments, disclosed is a compound of Formula (II). In some embodiments, disclosed is a pharmaceutically acceptable salt of the compound of Formula (II).

In some embodiments, disclosed are lipid nanoparticles (LNPs) comprising the compound of Formula (I), Formula (III) or any subgenus or species thereof, or a pharmaceutically acceptable salt thereof. In some embodiments, disclosed are lipid nanoparticles (LNPs) comprising the compound of Formula (I), Formula (III), or any subgenus or species thereof. The term “lipid nanoparticle” includes an electron dense nanostructural core produced by microfluidic mixing of lipid-containing solutions in ethanol with aqueous solutions. The lipid nanoparticles disclosed herein may be constructed from any materials used in conventional nanoparticle technology, for example, ionizable lipids, neutral lipids, sterols and polymer-conjugated lipids, provided that the net charge of the nanoparticle is about zero.

In some embodiments, the compound of Formula (I), Formula (III), or any subgenus or species thereof, is the ionizable lipid. Other non-limiting examples of ionizable lipids that may be combined with the compound of Formula (I), Formula (III), or any subgenus or species thereof in a lipid nanoparticle include, for instance, lipids containing a positive charge at the acidic scale of physiological pH range, for example 1,2-dilinoleyloxy-3-dimethylaminopropane (DLin-DMA), dilinoleylmethyl-4-dimethylaminobutyrate (DLin-MC3-DMA, (see e.g., U.S. Pat. No. 8,158,601), 2-dilinoleyl-4-dimethylaminoethyl-[1,3]-dioxolane (DLin-KC2-DMA), Merck-32 (see e.g., WO 2012/018754), Acuitas-5 (see e.g., WO 2015/199952), KL-10 (see e.g., U.S. Patent Application Publication 2012/0295832), C12-200 (see e.g., Love, K T et al., PNAS, 107: 1864 (2009)), and the like. The ionizable lipids may be present in an amount ranging from about 5% to about 90%, such as from about 10% to about 80%, for instance from about 25% to about 75%, for example, from about 40% to about 60%, from about 40% to about 50%, such as about 45% or about 50%, molar percent, relative to the total lipid present in the lipid nanoparticles.

The term “neutral lipid” includes lipids that have a zero-net charge at physiological pH, for example, lipids that exist in an uncharged form or neutral zwitterionic form at physiological pH, such as distearoyl phosphatidylcholine (DSPC), dioleoyl phosphatidylethanolamine (DOPE), dipalmitoyl phosphatidylcholine (DPPC), dimyristoyl phosphatidylcholine (DMPC), and the like, and combinations thereof. The neutral lipids may be present in an amount ranging from about 1% to about 50%, such as from about 5% to about 20%, for example, 7.5% to about 12.5%, for instance, about 10%, molar percent, relative to the total lipid present in the lipid nanoparticles. In some embodiments, the neutral lipid is DSPC. In some embodiments, the neutral lipid is DOPE. In some embodiments, the neutral lipid is DPPC. In some embodiments, the neutral lipid is DMPC.

The term “sterol” includes cholesterol, and the like. The sterols may be present in an amount ranging from about 10% to about 90%, such as from about 20% to about 50%, for instance, from about 35%-45%, such as about 38.5%, molar percent, relative to the total lipid present in the lipid nanoparticles. In some embodiments, the sterol is cholesterol.

The term “polymer-conjugated lipid” includes lipids that comprise a lipid portion and a polymer portion, such as pegylated lipids comprising both a lipid portion and a polyethylene glycol portion. Non-limiting examples include dimyristoyl phosphatidyl ethanolamine-poly(ethylene glycol) 2000 (DMPE-PEG2000), DPPE-PEG2000, DMG-PEG2000, DPG-PEG2000, PEG2000-c-DOMG, PEG2000-c-DOPG, and the like. The molecular weight of the poly(ethylene glycol) that may be used may range from about 500 and about 10,000 Da, or from about 1,000 to about 5,000 Da. In some embodiments, the polymer-conjugated lipid is DMPE-PEG2000. In some embodiments, the polymer-conjugated lipid is DPPE-PEG2000. In some embodiments, the polymer-conjugated lipid is DMG-PEG2000. In some embodiments, the polymer-conjugated lipid is DPG-PEG2000. In some embodiments, the polymer-conjugated lipid is PEG2000-c-DOMG. In some embodiments, the polymer-conjugated lipid is PEG2000-c-DOPG. The polymer-conjugated lipids may be present in an amount ranging from about 0% to about 20%, for example about 0.5% to about 5%, such as about 1% to about 2%, for instance, about 1.5%, molar percent, relative to the total lipid present in the lipid nanoparticles.

In at least one embodiment of the present disclosure, lipid nanoparticles may be prepared by combining multiple lipid components. For example, the lipid nanoparticles may be prepared combining the compound of Formula (I), Formula (III) or any subgenus or species thereof, or a pharmaceutically acceptable salt thereof, a sterol, a neutral lipid, and a polymer-conjugated lipid at a molar ratio of 50:40-x:10:x, with respect to the total lipids present. For example, the lipid nanoparticles may be prepared combining the compound of Formula (I), Formula (III) or any subgenus or species thereof, or a pharmaceutically acceptable salt thereof, a sterol, a neutral lipid, and a polymer-conjugated lipid at a molar ratio of 50:37:10:3 (mol/mol), or, for instance, a molar ratio of 50:38.5:10:1.5 (mol/mol), or, for example, 50:39.5:10:0.5 (mol/mol), or 50:39.75:10:0.25 (mol/mol).

In another embodiment, a lipid nanoparticle may be prepared using the compound of Formula (I), Formula (III) or any subgenus or species thereof, or a pharmaceutically acceptable salt thereof, a sterol (such as cholesterol), a neutral lipid (such as DSPC), and a polymer conjugated lipid (such as DMPE-PEG2000) at a molar ratio of about 50:38.5:10:1.5 (mol/mol), with respect to the total lipids present. Yet another non limiting example is a lipid nanoparticle comprising the compound of Formula (I), Formula (III) or any subgenus or species thereof, or a pharmaceutically acceptable salt thereof, a sterol (such as cholesterol), a neutral lipid (such as DSPC), and a polymer conjugated lipid (such as DMPE-PEG2000) at a molar ratio of about 47.7:36.8:12.5:3 (mol/mol), with respect to the total lipids present. Another non-limiting example is a lipid nanoparticle comprising the compound of Formula (I), Formula (III) or any subgenus or species thereof, or a pharmaceutically acceptable salt thereof, a sterol (such as cholesterol), a neutral lipid (such as DSPC), and a polymer conjugated lipid (such as DMPE-PEG2000) at a molar ratio of about 52.4:40.4:6.4:0.8 (mol/mol), with respect to the total lipids present. In another embodiment, a non-limiting example is a lipid nanoparticle comprising the compound of Formula (I), Formula (III) or any subgenus or species thereof, or a pharmaceutically acceptable salt thereof, a sterol (such as cholesterol), a neutral lipid (such as DSPC), and a polymer conjugated lipid (such as DMPE-PEG2000) at a molar ratio of about 53.5:41.2:4.6:0.7 (mol/mol), with respect to the total lipids present. Another non-limiting example is a lipid nanoparticle comprising the compound of Formula (I), Formula (III) or any subgenus or species thereof, or a pharmaceutically acceptable salt thereof, a sterol (such as cholesterol), a neutral lipid (such as DSPC), and a polymer conjugated lipid (such as DMPE-PEG2000) at a molar ratio of about 30:50:19:1 (mol/mol), with respect to the total lipids present.

The selection of neutral lipids, sterols, and/or polymer-conjugated lipids that comprise the lipid nanoparticles, as well as the relative molar ratio of such lipids to each other, may be determined by the characteristics of the selected lipid(s), the nature of the intended target cells, and the characteristics of the nucleic acid segment to be delivered. For instance, in certain embodiments, the molar percent of the compound of Formula (I), Formula (III) or any subgenus or species thereof, or a pharmaceutically acceptable salt thereof, in the lipid nanoparticle may be greater than about 10%, greater than about 20%, greater than about 30%, greater than about 40%, greater than about 50%, greater than about 60%, or greater than about 70%, relative to the total lipids present. The molar percent of neutral lipid in the lipid nanoparticle may be greater than about 5%, greater than about 10%, greater than about 20%, greater than about 30%, or greater than about 40%, relative to the total lipids present. The molar percent of sterol in the lipid nanoparticle may be greater than about 10%, greater than about 20%, greater than about 30%, or greater than about 40%, relative to the total lipids present. The molar percent of polymer-conjugated lipid in the lipid nanoparticle may be greater than about 0.25%, such as greater than about 1%, greater than about 1.5%, greater than about 2%, greater than about 5%, or greater than about 10%, relative to the total lipids present.

According to the present disclosure, the lipid nanoparticles may comprise each of the compound of Formula (I), Formula (III), or any subgenus or species thereof, or a pharmaceutically acceptable salt thereof, neutral lipids, sterols, and/or polymer-conjugated lipids in any useful orientation desired. For example, the core of the nanoparticle may comprise the compound of Formula (I), Formula (III), or any subgenus or species thereof, or a pharmaceutically acceptable salt thereof, alone or in combination with another ionizable lipid, a sterol and one or more layers comprising neutral lipids and/or polymer-conjugated lipids may subsequently surround the core. For instance, according to one embodiment, the core of the lipid nanoparticle may comprise a core comprising the compound of Formula (I), Formula (III), or any subgenus or species thereof, or a pharmaceutically acceptable salt thereof, and a sterol (e.g., cholesterol) in any particular ratio, surrounded by a neutral lipid monolayer (e.g., DSPC) of any particular thickness, further surrounded by an outer polymer-conjugated lipid monolayer of any particular thickness. In such examples, the nucleic acid segment may be incorporated into any one of the core or subsequent layers depending upon the nature of the intended target cells, and the characteristics of the nucleic acid segment to be delivered. The core and outer layers may further comprise other components typically incorporated into lipid nanoparticles known in the art. Furthermore, it is understood by one skilled in the art that liposomes are delivery vehicles that possess a vesicular structure distinct from the lipid nanoparticles as disclosed herein. The liposome vesicles are composed of a lipid bilayer that forms in the shape of a hollow sphere encompassing an aqueous phase. For example, liposomes contain the lamellar phase while the lipid nanoparticles have non-lamellar structures.

In addition, the molar percent of the components of the lipid nanoparticle (e.g., the compound of Formula (I), Formula (III), or any subgenus or species thereof, or a pharmaceutically acceptable salt thereof, neutral lipids, sterols, and/or polymer-conjugated lipids) that comprise the lipid nanoparticles may be selected in order to provide a particular physical parameter of the overall lipid nanoparticle, such as the surface area of one or more of the lipids. For example, the molar percent of the compound of Formula (I), Formula (III), or any subgenus or species thereof, or a pharmaceutically acceptable salt thereof, neutral lipids, sterols, and/or polymer-conjugated lipids that comprise the lipid nanoparticles may be selected to yield a surface area per neutral lipid, for example, DSPC. By way of non-limiting example, the molar percent of the compound of Formula (I), Formula (III) or any subgenus or species thereof, or a pharmaceutically acceptable salt thereof, neutral lipids, sterols, and/or polymer-conjugated lipids may be determined to yield a surface area per DSPC of about 1.0 nm² to about 2.0 nm², for example about 1.2 nm².

According to the present disclosure, the lipid nanoparticles may further comprise a nucleic acid segment, which may be associated on the surface of the lipid nanoparticles and/or encapsulated within the same lipid nanoparticles.

The term “nucleic acid segment” is understood to mean any one or more nucleic acid segments selected from antisense oligonucleotides, DNA, mRNAs, siRNAs, Cas9guided-RNA complex, or combinations thereof. The nucleic acid segments herein may be wildtype or modified. In at least one embodiment, the lipid nanoparticles may comprise a plurality of different nucleic acid segments. In yet another embodiment, the nucleic acid segment, wildtype or modified, encodes a polypeptide of interest. A modified nucleic acid segment includes nucleic acid segments with chemical modifications to any part of the structure such that the nucleic acid segment is not naturally occurring. In some embodiments, the nucleic acid segment is an RNA. In some embodiments, the nucleic acid segment is an mRNA. In some embodiments, the nucleic acid segment is a modified mRNA.

The term “therapeutically effective amount” as used herein refers to an amount of nucleic acid segment sufficient to modulate protein expression in a target tissue and/or cell type. In some embodiments, a therapeutically effective amount of the nucleic acid segment is an amount sufficient to treat a disease or disorder associated with the protein expressed by the nucleic acid segment.

In at least one embodiment, the weight ratio of total lipid phase to nucleic acid segment ranges from about 40:1 to about 1:1, such as about 10:1. This corresponds to an approximate molar ratio of the compound of Formula (I), Formula (III) or any subgenus or species thereof, or a pharmaceutically acceptable salt thereof, to nucleic acid monomer of about 3:1. In yet another example, the weight ratio of total lipid phase to nucleic acid segment ranges from about 30:1 to about 1:1, such as about 20:1, which corresponds to an approximate molar ratio of the compound of Formula (I), Formula (III) or any subgenus or species thereof, or a pharmaceutically acceptable salt thereof, to nucleic acid monomer of about 6:1. However, the relative molar ratio of lipid phase and/or lipid phase components to the nucleic acid monomer may be determined by the nature of the intended target cells and characteristics of nucleic acid segment and thus, are not limited in scope to the above-identified embodiments. In some embodiments, the molar ratio of the compound of Formula (I), Formula (III) or any subgenus or species thereof, or a pharmaceutically acceptable salt thereof, to nucleic acid monomer is from about 2.75:1 to 6:1. In some embodiments, the molar ratio of the compound of Formula (I), Formula (III) or any subgenus or species thereof, or a pharmaceutically acceptable salt thereof, to nucleic acid monomer is about 2.75:1. In some embodiments, the approximate molar ratio of the compound of Formula (I), Formula (III) or any subgenus or species thereof, or a pharmaceutically acceptable salt thereof, to nucleic acid monomer of about 3:1. In some embodiments, the molar ratio of the compound of Formula (I), Formula (III) or any subgenus or species thereof, or a pharmaceutically acceptable salt thereof, to nucleic acid monomer is about 5.5:1. In some embodiments, the approximate molar ratio of the compound of Formula (I), Formula (III) or any subgenus or species thereof, or a pharmaceutically acceptable salt thereof, to nucleic acid monomer of about 6:1.

In some embodiments, the lipid nanoparticles have a z-average particle diameter (<d>_{Z}) of about 200 nm or less, for example, less than or equal to about 100 nm, or, for instance, less than or equal to about 75 nm. In at least one embodiment of the present disclosure, the lipid nanoparticles have a z-average particle diameter ranging from about 50 nm to about 100 nm, for example, about 60 nm to about 90 nm, from about 70 nm to about 80, such as about 75 nm.

In certain embodiments, the lipid nanoparticles have an encapsulation efficiency (% EE) of nucleic acid segments of about 80% or higher, such as higher than about 90%, such as ranging from about 95%-100%. As used herein, the term “encapsulation efficiency” refers to the ratio of encapsulated nucleic acid segment in the lipid nanoparticles to total nucleic acid segment content in the lipid nanoparticle composition measured by lysis of the lipid nanoparticles using a detergent, e.g., Triton X-100.

Pharmaceutical compositions of the present disclosure may further comprise at least one pharmaceutically acceptable carrier. As used herein, the term “pharmaceutically acceptable carrier” includes compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The pharmaceutical compositions may be in a form suitable for parenteral administration. The pharmaceutical compositions may be in a form suitable for intratracheal instillation, bronchial instillation, and/or inhalation. Pharmaceutical liquid compositions can be nebulized by use of inert gases. Nebulized suspensions may be breathed directly from the nebulizing device or the nebulizing device can be attached to face masks tent, or intermittent positive pressure breathing machine.

The amount of nucleic acid segment that is combined with one or more pharmaceutically acceptable carriers to produce a single dosage form will necessarily vary depending upon the subject treated and the particular route of administration. For further information on routes of administration and dosage regimes the reader is referred to Chapter 25.3 in Volume 5 of Comprehensive Medicinal Chemistry (Corwin Hansch; Chairman of Editorial Board), Pergamon Press 1990.

In one embodiment, the present disclosure provides a method for administering pharmaceutical compositions comprising a plurality of lipid nanoparticles comprising the compound of Formula (I), Formula (III) or any subgenus or species thereof, or a pharmaceutically acceptable salt thereof, and a therapeutically effective amount of a nucleic acid segment in a subject in need thereof.

The term “subject” includes warm-blooded mammals, for example, primates, cows, pigs, sheep, dogs, cats, rabbits, rats, and mice. In some embodiments, the subject is a primate, for example, a human. In some embodiments, the subject is in need of treatment (e.g., the subject would benefit biologically or medically from treatment).

The lipid nanoparticles disclosed herein may further serve as platforms for selective delivery of nucleic acid segments to target cells and tissues, such as antisense oligonucleotides, DNA, mRNAs, siRNAs, Cas9-guideRNA complex. Thus, in one embodiment, is a method of delivering a nucleic acid segment to a cell comprising contacting the cell, in vitro or in vivo, with a pharmaceutical composition comprising a plurality of lipid nanoparticles comprising the compound of Formula (I), Formula (III) or any subgenus or species thereof, or a pharmaceutically acceptable salt thereof, and a therapeutically effective amount of a nucleic acid segment. In some embodiments, the nucleic acid segment modulates expression, for example, by increasing or decreasing expression, or by upregulating or downregulating expression of the polypeptide.

Another embodiment provides a method for delivering a therapeutically effective amount of a nucleic acid segment to a subject in need thereof, comprising administering to the subject a pharmaceutical composition comprising a plurality of lipid nanoparticles comprising the compound of Formula (I), Formula (III) or any subgenus or species thereof, or a pharmaceutically acceptable salt thereof, and a therapeutically effective amount of a nucleic acid segment.

The pharmaceutical compositions comprising a plurality of lipid nanoparticles comprising the compound of Formula (I), Formula (III) or any subgenus or species thereof, or a pharmaceutically acceptable salt thereof, and a nucleic acid segment disclosed herein may be used to treat a wide variety of disorders and diseases characterized by underexpression of a polypeptide in a subject, overexpression of a polypeptide in a subject, and/or absence/presence of a polypeptide in a subject. Accordingly, disclosed are methods of treating a subject suffering from a disease or disorder comprising administering to the subject a pharmaceutical composition comprising a plurality of lipid nanoparticles comprising the compound of Formula (I), Formula (III) or any subgenus or species thereof, or a pharmaceutically acceptable salt thereof, and a therapeutically effective amount of a nucleic acid segment.

Further disclosed is the use of a pharmaceutical composition comprising a plurality of lipid nanoparticles comprising the compound of Formula (I), Formula (III) or any subgenus or species thereof, or a pharmaceutically acceptable salt thereof, and a therapeutically effective amount of a nucleic acid segment, to treat a disease or disorder.

Further disclosed is a pharmaceutical composition for use in the treatment of a disease or disorder, wherein the pharmaceutical composition comprises a plurality of lipid nanoparticles comprising the compound of Formula (I), Formula (III) or any subgenus or species thereof, or a pharmaceutically acceptable salt thereof, and a therapeutically effective amount of a nucleic acid segment.

Further disclosed are methods for increasing protein expression in cells, comprising administering a pharmaceutical composition comprising a plurality of lipid nanoparticles comprising the compound of Formula (I), Formula (III) or any subgenus or species thereof, or a pharmaceutically acceptable salt thereof, and a nucleic acid segment to a subject in need thereof. In at least one embodiment, protein expression may be increased by a factor of about 2 up to 24 hours. In another embodiment, protein expression may be increased by a factor of about 3 up to 72 hours.

### EXAMPLES

### General Methods

¹H NMR: 300 MHz; probe: 5 mm broadband liquid probe BBFO with ATM+Z PABBO BB-1H/D; magnet: ULTRASHIELD™300; Crate: AVANCE III 300; Auto Sampler: SampleXpress™60; software: Topspin 3. 400 MHz; probe: 5 mm Broadband liquid probe BBFO with ATM+Z PABBO BB-1H/D; magnet ASCEND™400; crate AVANCE III 300; auto sampler SampleXpress™60; software: Topspin 3. All spectra were calibrated with TMS as internal reference.

500 MHz; probe: 5 mm Bruker Smart probe with ATM+Z PABBO 500S1-BBF-H-D; magnet: ASCEND™ 500; Console: AVANCE Neo 500; Auto Sampler: SampleXpress™60; software: Topspin 4. Proton chemical shifts are expressed in parts per million (ppm, δ scale) and are referenced to residual protium in the NMR solvent (Chloroform-d: δ 7.26, Methanol-d4: δ 3.31, DMSO-d6: δ 2.50).

Data are represented as follows: chemical shift, multiplicity (s=singlet, d=doublet, t=triplet, q=quartet, dd=doublet of doublets, dt=doublet of triplets, m=multiplet, br=broad, app=apparent), integration, and coupling constant (J) in Hertz (Hz).

LCMS: Instrument Shimadzu LCMS-2020 coupled with DAD detector, ELSD detector and 2020EV MS; column Shim-pack XR-ODS C18 (50×3.0 mm, 2.2 μm); eluent A water (0.05% TFA), eluent B MeCN (0.05% TFA); gradient 5-95% B in 2.00 min, hold 0.70 min (method A) or 60-95% B in 1.00 min, hold 1.70 min (method B); flow 1.20 mL/min; PDA detection (SPD-M20A) 190-400 nm. Mass spectrometer in ESI mode.

Method C: UPLC-MS was carried out using a Waters Acquity UPLC and Waters SQD mass spectrometer (column temp 30° C., UV detection=210-400 nm, mass spec=ESI with positive/negative switching) at a flow rate of 1 mL/min using a solvent gradient of 2 to 98% B over 1.5 mins (total runtime with equilibration back to starting conditions 2 min), where A=0.1% formic acid in water and B=0.1% formic acid in acetonitrile (for acid work) or A=0.1% ammonium hydroxide in water and B=acetonitrile (for base work). For acid analysis the column used was Waters Acquity HSS T3, 1.8 mm, 2.1×30 mm, for base analysis the column used was Waters Acquity BEH C18, 1.7 mm, 2.1×30 mm.

HPLC: Instrument Shimadzu LCMS-2020 coupled with a DAD detector, CAD detector; column Ascentis Express C18 (100×4.6 mm), 2.7 μm; mobile phase A water (0.05% TFA), mobile phase B MeCN; gradient 10-95% B in 4.00 min, hold 8 min, or as indicated, flow 1.50 mL/min; purity as area %.

Preparative HPLC: instruments Waters 2545 Binary Gradient module, Waters 2767 Sample Manager, Waters 2489 UV/Visible Detector, Waters SQ Detector 2. Method A: column XSelect CSH Prep C18 OBD Column, 19×250 mm, 5 μm; mobile phase A water (0.05% TFA), mobile phase B MeCN; flow rate 25 mL/min, gradient as indicated. Method B: column SunFire C18 OBD, 19×250 mm, 5 μm; mobile phase A water (0.05% TFA), mobile phase B MeCN; flow rate 60 mL/min, gradient as indicated.

### Abbreviations

1,2-DCE 1,2-dichloroethane

DCM dichloromethane

DMSO dimethylsulfoxide

DIEA N,N-diisopropylethylamine

DMAP N,N-dimethylaminopyridine

<d>_{N} number-average particle diameter

<d>_{Z} z-average particle diameter

EDCI 1-Ethyl-3-(3-dimethylaminopropyl)carbodiimide

EE % encapsulation efficacy

HPLC High-performance liquid chromatography

NMP N-methyl-2-pyrrolidone

PDI polydispersity index

PE petrolether (30-50)

PBS phosphate-buffered saline

rt room temperature

THF tetrahydrofuran

Z-pot zeta-potential

Scheme 1 below demonstrates the synthetic procedures for preparing Examples 1 to 5.

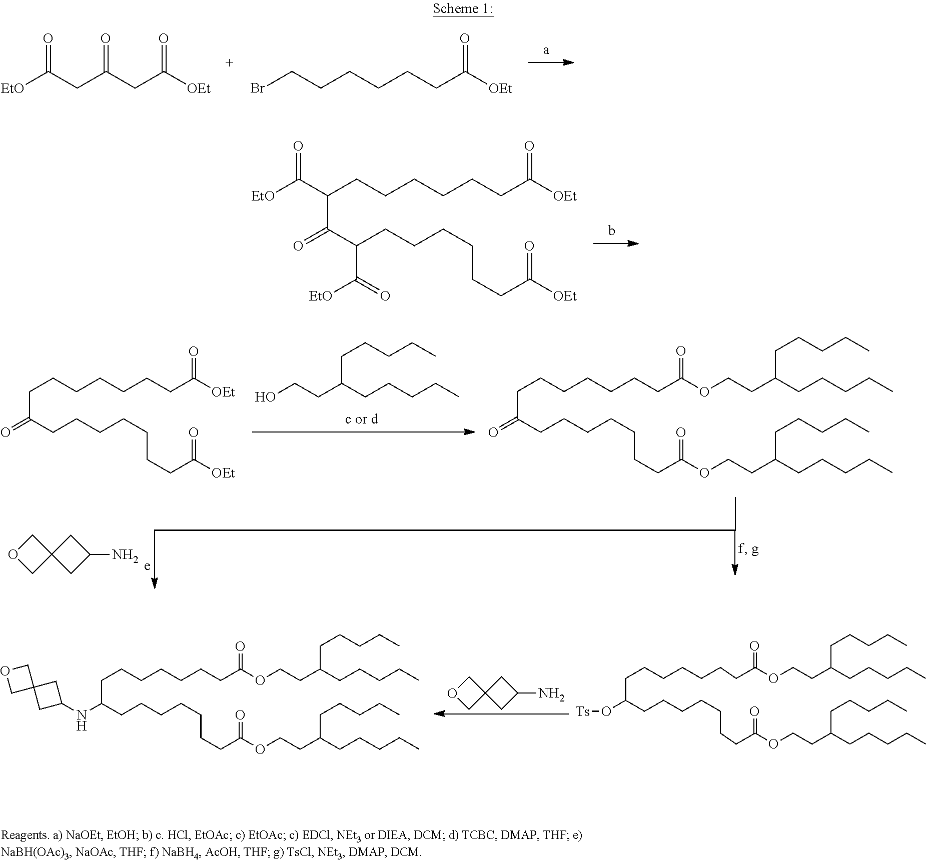

### Example 1. Synthesis of Compound 1

### Intermediate 1: Bis(3-pentyloctyl) 9-oxoheptadecanedioate

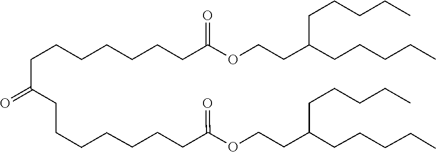 [SMILES: CCCCCC(CCCCC)CCOC(=O)CCCCCCCC(=O)CCCCCCCC(=O)OCCC(CCCCC)CCCCC]

### Step 1: Tetraethyl 8-oxopentadecane-1,7,9,15-tetracarboxylate

Sodium ethanolate (16.8 g, 247 mmol) was added in one portion to diethyl 3-oxopentanedioate (25 g, 124 mmol) dissolved in ethanol (200 mL) at rt under nitrogen. The resulting mixture was stirred at 80° C. for 1 h, followed by addition of ethyl 7-bromoheptanoate (103 g, 432.73 mmol). The reaction mixture was heated to reflux overnight, thereafter concentrated and diluted with EtOAc (200 mL) and washed twice with water (200 mL), dried over sodium sulfate, filtered and evaporated to afford crude product. Purification by flash chromatography on silica gel (elution by 0 to 20% EtOAc in PE). Pure fractions were evaporated to dryness to afford tetraethyl 8-oxopentadecane-1,7,9,15-tetracarboxylate (50.0 g, 79%) as an orange oil. ¹H NMR (300 MHz, DMSO-d6) δ 4.08 (m, 8H), 3.44-3.28 (m, 2H), 2.26 (m, 4H), 1.82-1.65 (m, 4H), 1.61-1.41 (m, 8H), 1.35-1.13 (m, 20H).

### Step 2: 9-Oxoheptadecanedioic acid

Tetraethyl 8-oxopentadecane-1,7,9,15-tetracarboxylate (48 g, 93.3 mmol) was added in one portion to conc. HCl (36%, 400 mL) and HOAc (200 mL) at 25° C. under nitrogen. The resulting mixture was stirred under reflux for 15 h. The reaction mixture was cooled to rt and poured into water. The precipitate was collected by filtration. Recrystallization from acetone afforded 9-oxoheptadecanedioic acid (11.0 g, 38%) as a white solid. ¹H NMR (300 MHz, DMSO-d6) δ 11.95 (br. s, 2H), 2.38 (t, J=7.3 Hz, 4H), 2.18 (t, J=7.3 Hz, 4H), 1.56-1.37 (m, 8H), 1.24 (q, J=4.7 Hz, 12H).

### Step 3: Bis(3-pentyloctyl) 9-oxoheptadecanedioate (Intermediate 1)

To a solution of 9-oxoheptadecanedioic acid (19.2 g, 61.03 mmol), 3-pentyloctan-1-ol (see WO 2013/086354, p191 for the procedures of preparing 3-pentyloctan-1-ol) (26.3 g, 131 mmol) and DIEA (32.0 mL, 183 mmol) in DCM (250 mL) was added EDCI (29.3 g, 153 mmol) and DMAP (7.46 g, 61.0 mmol). The mixture was stirred for 15 h, quenched with water (50 mL). After addition of EtOAc (750 mL) the mixture was washed twice with each 2M HCl (100 mL), water (100 mL), and brine (100 mL), dried over sodium sulfate and evaporated. The residue was purified by flash chromatography (eluent 0-5% EtOAc in PE) to afford bis(3-pentyloctyl) 9-oxoheptadecanedioate (Intermediate 1, (25.1 g, 61%) as a pale yellow oil. ¹H NMR (300 MHz, CDCl₃) δ 4.07 (t, J=7.1 Hz, 4H), 2.37 (t, J=7.4 Hz, 4H), 2.27 (t, J=7.5 Hz, 4H), 1.68-1.48 (m, 12H), 1.45-1.15 (m, 46H), 0.88 (t, J=6.8 Hz, 12H).

### Compound 1: Bis(3-pentyloctyl) 9-((2-oxaspiro[3.3]heptan-6-yl)amino)heptadecanedioate

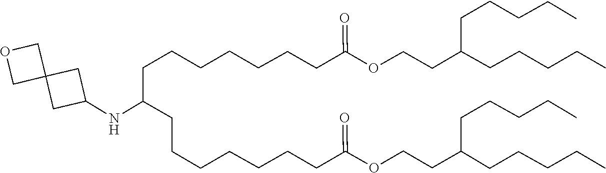 [SMILES: CCCCCC(CCCCC)CCOC(=O)CCCCCCCC(CCCCCCCC(=O)OCCC(CCCCC)CCCCC)NC1CC2(COC2)C1]

Sodium triacetoxyborohydride (3.28 g, 15.46 mmol) was added to 2-oxaspiro[3.3]heptan-6-amine hydrochloride (2.313 g, 15.46 mmol) and bis(3-pentyloctyl) 9-oxoheptadecanedioate (3.5 g, 5.15 mmol) in DCE (30 mL) and NMP (12.00 mL). The resulting mixture was stirred at rt for 15 hours.

The reaction mixture was concentrated and diluted with EtOAc (150 mL), and washed sequentially with water (3×25 mL) and brine (3×25 mL). The organic layer was dried over Na₂SO₄, filtered and evaporated to afford 3 g of crude product containing solvent as orange oil. The crude product was purified by flash chromatography on silica gel, elution gradient 0 to 20% MeOH in DCM, to afford 3 g of crude product as yellow oil. The crude product was purified by preparative HPLC (A:Water (10 mM NH₄HCO₃), B:CAN, 90-95% B in 7 min; Flow: 25 mL/min) and afforded after freeze drying 1.025 g (26%) of the title compound (Compound 1) as light yellow oil.

LCMS m/z 776.6 [M+H]⁺, t_{R} 2.060 min (method B). HPLC purity 96.4% t_{R} 9.301 (B 30-95% in 8.00 min, hold 4 min). ¹H NMR (300 MHz, CD₃OD, 23° C.) δ 4.74 (s, 2H), 4.60 (s, 2H), 4.13 (t, 4H), 3.09-3.26 (m, 1H), 2.42-2.63 (m, 3H), 2.33 (t, 4H), 1.98 (ddd, 2H), 1.56-1.7 (m, 8H), 1.33 (m, 54H), 0.88-0.99 (t, 12H). Expected Number of Hs: 93; assigned Hs: 92.

### Example 2. Synthesis of Compound 2: (bis(3-pentyloctyl) 9-[(oxan-4-yl)amino]heptadecanedioate)

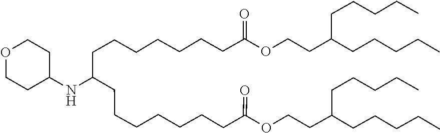 [SMILES: CCCCCC(CCCCC)CCOC(=O)CCCCCCCC(CCCCCCCC(=O)OCCC(CCCCC)CCCCC)NC1CCOCC1]

Compound 2 was prepared following the protocol described for Compound 1, starting from tetrahydro-2H-pyran-4-amine (156 mg, 1.55 mmol) and bis(3-pentyloctyl) 9-oxoheptadecanedioate (350 mg, 0.52 mmol). Purification by flash chromatography on silica gel (0-10% MeOH in DCM), followed by preparative HPLC: XSelect CSH F-phenyl OBD, 19*250 mm, 5 μm; A: water (0.05% TFA), B: ACN; Flow rate: 25 mL/min; gradient: 56 to 95% B in 7 min. The crude product was isolated as a pale yellow oil (232 mg, 59%). LCMS m/z 764.8 [M+H]+, t_{R} 2.077 min (method A). HPLC purity 93.8%, t_{R} 9.427 min (10-95% B in 8 min, hold 4 min).

¹H NMR (300 MHz, MeOD) δ 4.12 (4H, t), 4.05 (2H, dd), 3.48 (3H, t), 2.34 (4H, t), 2.02 (2H, m), 1.54-1.78 (14H, m), 1.21-1.53 (51H, m), 0.88-0.99 (12H, m). Expected Number of Hs: 93; assigned Hs: 92.

Compound 2 (232 mg crude), obtained as described above, was dissolved in EtOH and further purified by preparative SFC: Stationary phase: DCPak PBT, 250*20 mm ID, 5 μm; Mobile phase: A: CO₂, B: 20 mM NH₃ in MeOH; Flow rate: 100 g/min; Gradient 12% B (3.5 min), 12-35% B (1 min), 35% B (4 min); Pressure: 120 bar Temperature: 40° C. Yield: 110 mg.

SFC-CAD purity 99%, t_{R} 2.432 min. Stationary phase: DCPak PBT, 150*4.6 mm ID, 5 μm; Mobile phase: A: CO₂ B: 20 mM NH₃ in MeOH; Flow rate 3.5 mL/min; Gradient 5-40% B (5 min), 40% B (1 min); Pressure: 120 bar; Temperature: 40° C.

### Example 3. Synthesis of Compound 3: (bis(3-pentyloctyl) 9-(((tetrahydrofuran-3-yl)methyl)amino)heptadecanedioate)

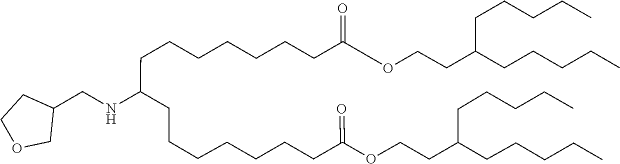 [SMILES: CCCCCC(CCCCC)CCOC(=O)CCCCCCCC(CCCCCCCC(=O)OCCC(CCCCC)CCCCC)NCC1CCOC1]

Compound 3 was prepared following the protocol described for Compound 1 using Intermediate 1. Sodium triacetoxyhydroborate (32.9 mg, 0.16 mmol) was added in one portion to a stirred solution of bis(3-pentyloctyl) 9-oxoheptadecanedioate (58.6 mg, 0.09 mmol), (tetrahydrofuran-3-yl)methanamine (14.25 μl, 0.13 mmol) and acetic acid (181 μl, 0.18 mmol) in 1,2-DCE (2 mL) and NMP (0.5 mL) under argon. The resulting solution was stirred at 25° C. for 18 hours. The reaction mixture was diluted with DCM (15 mL), water (5 mL) and sat. Na₂CO₃ (5 mL). The layers were separated, and the aqueous layer was extracted three times with DCM (15 mL). The combined organic layers were dried over MgSO₄, filtered and evaporated to dryness to afford crude product. The resulting residue was purified by flash silica chromatography, elution gradient 10 to 50% MeOH in DCM (w/1% NH₄OH). Product fractions were concentrated under reduced pressure to dryness to afford Compound 3 (0.017 g, 26.1%) as a colorless oil. ¹H NMR (500 MHz, Methanol-d4) δ ppm 0.9 (t, J=7.1 Hz, 12H) 1.3-1.4 (m, 48H) 1.4-1.5 (m, 6H) 1.5-1.7 (m, 9H) 2.0-2.1 (m, 1H) 2.3-2.3 (m, 4H) 2.3-2.4 (m, 1H) 2.5 (br t, J=5.9 Hz, 1H) 2.6-2.6 (m, 2H) 3.4-3.5 (m, 1H) 3.7-3.8 (m, 1H) 3.8-3.9 (m, 2H) 4.1 (t, J=6.8 Hz, 4H); C₄₈H₉₃NO₅ m/z calcd. 763.705 observed 764.8 [M+H]⁺ (LCMS—Method C).

### Example 4. Synthesis of Compound 4: (bis(3-pentyloctyl) 9-(((tetrahydro-2H-pyran-4-yl)methyl)amino)heptadecanedioate)

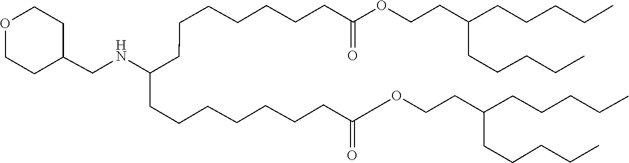 [SMILES: CCCCCC(CCCCC)CCOC(=O)CCCCCCCC(CCCCCCCC(=O)OCCC(CCCCC)CCCCC)NCC1CCOCC1]

Compound 4 was prepared following the protocol described for Compound 1 using Intermediate 1. Sodium triacetoxyhydroborate (35.2 mg, 0.17 mmol) was added in one portion (after 10 min) to a stirred solution of bis(3-pentyloctyl) 9-oxoheptadecanedioate (62.7 mg, 0.09 mmol), (tetrahydro-2H-pyran-4-yl)methanamine (15.64 μl, 0.14 mmol) and acetic acid (194 μl, 0.19 mmol) in 1,2-DCE (2 mL) and NMP (0.5 mL) under argon. The resulting solution was stirred at 25° C. for 40 hours. The reaction mixture was diluted with DCM (15 mL), water (5 mL) and sat. Na₂CO₃ (5 mL). The layers were separated, and the aqueous layer was extracted three times with DCM (3×15 mL). The combined organic layers were dried over MgSO₄, filtered and concentrated under reduced pressure to dryness to afford crude product. The resulting residue was purified by flash silica chromatography, elution gradient 10 to 45% MeOH in DCM (w/1% NH₄OH). Product fractions were concentrated under reduced pressure to dryness to afford Compound 4 (0.034 g, 47.7%) as a colorless oil. ¹H NMR (500 MHz, Methanol-d4) δ ppm 0.9 (t, J=7.0 Hz, 12H) 1.2-1.4 (m, 52H) 1.4-1.5 (m, 6H) 1.5-1.8 (m, 9H) 2.3 (t, J=7.3 Hz, 4H) 2.5-2.6 (m, 3H) 3.4-3.5 (m, 2H) 3.9 (dd, J=11.0, 4.0 Hz, 2H) 4.1 (t, J=6.7 Hz, 4H); C₄₉H₉₅NO₅ m/z calcd. 777.721 observed 778.7 [M+H]⁺ (LCMS—Method C).

### Example 5. Synthesis of Compound 5: (bis(3-pentyloctyl) 9-((oxetan-3-ylmethyl)amino)heptadecanedioate)

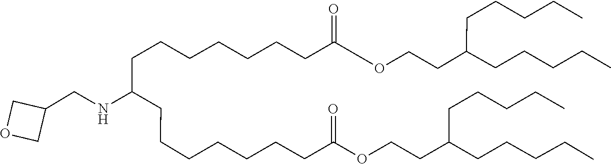 [SMILES: CCCCCC(CCCCC)CCOC(=O)CCCCCCCC(CCCCCCCC(=O)OCCC(CCCCC)CCCCC)NCC1COC1]

Compound 5 was prepared following the protocol described for Compound 1 using Intermediate 1. Sodium triacetoxyhydroborate (65.2 mg, 0.31 mmol) was added in one portion to a stirred solution of bis(3-pentyloctyl) 9-oxoheptadecanedioate (69.6 mg, 0.10 mmol) and oxetan-3-ylmethanaminium chloride (38.0 mg, 0.31 mmol) in 1,2-DCE (2 mL) and NMP (0.5 mL) under argon. The resulting solution was stirred at 25° C. for 50 hours. The reaction mixture was diluted with DCM (15 mL), water (5 mL) and sat. Na₂CO₃ (5 mL). The layers were separated, and the aqueous layer was extracted three times with DCM (15 mL). The combined organic layers were dried over MgSO₄, filtered and concentrated under reduced pressure to dryness to afford crude product. The resulting residue was purified by flash silica chromatography, elution gradient 10 to 50% MeOH in DCM (1% NH₄OH). Product fractions were concentrated under reduced pressure to dryness to afford bis(3-pentyloctyl) 9-((oxetan-3-ylmethyl)amino)heptadecanedioate (0.048 g, 62.8%) as a colorless oil. ¹H NMR (500 MHz, Methanol-d4) δ ppm 0.9 (t, J=7.1 Hz, 12H) 1.3-1.4 (m, 48H) 1.4-1.5 (m, 6H) 1.6-1.7 (m, 8H) 2.3 (t, J=7.4 Hz, 4H) 2.5 (t, J=5.9 Hz, 1H) 2.9 (d, J=7.5 Hz, 2H) 3.1-3.1 (m, 1H) 4.1 (t, J=6.8 Hz, 4H) 4.4 (t, J=6.0 Hz, 2H) 4.8-4.8 (m, 2H); C₄₇H₉₁NO₅ m/z calcd. 749.690 observed 750.6 [M+H]⁺ (LCMS—Method C).

Scheme 2 below illustrates the synthetic procedures for preparing Examples 6 to 9. In step d below, AcOH as an additive was used in reductive amination when utilizing the respective amines as a free base.

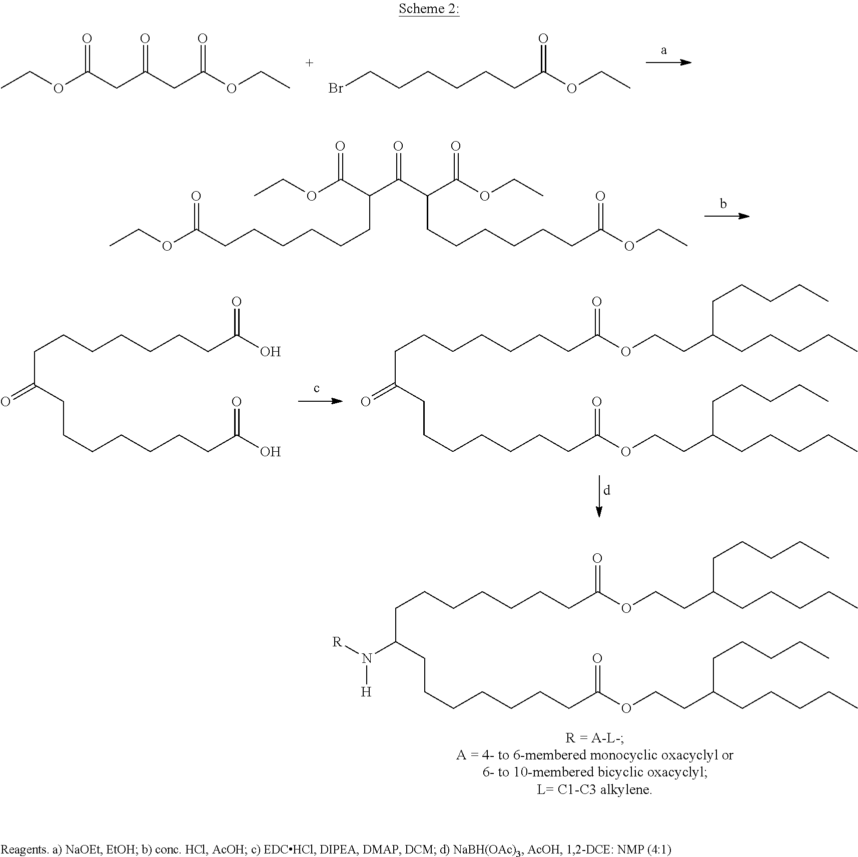

### Example 6. Synthesis of Compound 6: bis(3-pentyloctyl) 9-(((tetrahydro-2H-pyran-2-yl)methyl)amino)heptadecanedioate

Step a): sodium ethanolate (2.52 g, 37.09 mmol) was added portionwise to a stirred solution of diethyl 3-oxopentanedioate (4.49 ml, 24.73 mmol) in absolute (99.5%) ethanol (14 mL) at 25° C. over a period of 10 minutes under argon. The resulting suspension was stirred at 81° C. for 1 hour. To the reaction mixture ethyl 7-bromoheptanoate (12.05 ml, 61.82 mmol) was added dropwise and the suspension was stirred at 81° C. for a further 18 hours. The reaction mixture was cooled down to RT and concentrated under reduced pressure to dryness and redissolved in DCM (50 mL), and extracted 3 times with water (50 mL), and washed with saturated aq. NaCl (50 mL). The organic layer was dried over MgSO₄, filtered and concentrated under reduced pressure to afford crude product. The resulting residue was purified by flash silica chromatography, elution gradient 10 to 50% EtOAc in hexanes. Product fractions were concentrated under reduced pressure to dryness to afford tetraethyl 8-oxopentadecane-1,7,9,15-tetracarboxylate (8.10 g, 63.6%) as a pale yellow oil. ¹H NMR (500 MHz, Chloroform-d) δ ppm 1.2-1.4 (m, 24H) 1.5-1.9 (m, 8H) 2.2-2.3 (m, 4H) 3.4-3.5 (m, 2H) 4.1-4.2 (m, 8H).

Step b): hydrogen chloride (33.3 ml, 406.06 mmol) was added slowly to a stirred solution of tetraethyl 8-oxopentadecane-1,7,9,15-tetracarboxylate (8.1 g, 15.74 mmol) in acetic acid (20 mL) at 25° C. The resulting solution was stirred at 102° C. for 18 hours w/reflux condenser and an outlet to get rid of excess of HCl gas. Reaction was cooled down to RT and poured the reaction mixture on ice-water (50 mL) and was allowed to sit for 30 min. The precipitate was collected by filtration, washed with cold water (3×20 mL) and dried under vacuum to afford 9-oxoheptadecanedioic acid (crude) as a pale yellow solid. The crude product was purified by crystallization from acetone to afford 9-oxoheptadecanedioic acid (1.381 g, 27.9%) as a white solid. ¹H NMR (500 MHz, DMSO-d6) δ ppm 1.2 (br s, 12H) 1.4 (dt, J=18.6, 6.7 Hz, 8H) 2.2 (t, J=7.2 Hz, 4H) 2.3-2.4 (m, 4H) 11.8-12.1 (m, 2H); C₁₇H₃₀O₅ m/z calcd. 314.209 observed 313.1 [M−H]⁻ (LCMS).

Step c): 3-(((ethylimino)methylene)amino)-N,N-dimethylpropan-1-amine hydrochloride (690 mg, 3.60 mmol) was added in one portion to a stirred solution of 9-oxoheptadecanedioic acid (419 mg, 1.33 mmol), 3-pentyloctan-1-ol (see WO 2013/086354, p191 for the procedures of preparing 3-pentyloctan-1-ol) (641 mg, 3.20 mmol), N,N-dimethylpyridin-4-amine (32.6 mg, 0.27 mmol) and N-ethyl-N-isopropylpropan-2-amine (836 μl, 4.80 mmol) in DCM (20 mL) at 25° C. under argon. The resulting solution was stirred at 25° C. for 50 hours. The reaction mixture was diluted with DCM (25 mL), water (10 mL) and sat. aq. NH₄Cl (10 mL). The layers were separated, and the aqueous layer was extracted three times with DCM (25 mL). The combined organic layers were dried over MgSO₄, filtered and concentrated under reduced pressure to dryness to afford crude product. The resulting residue was purified by flash silica chromatography, elution gradient 10 to 40% EtOAc in hexanes. Product fractions were concentrated under reduced pressure to dryness to afford bis(3-pentyloctyl) 9-oxoheptadecanedioate (0.820 g, 91%) as a pale yellow oil. ¹H NMR (500 MHz, Chloroform-d) δ ppm 0.9-0.9 (m, 12H) 1.2-1.3 (m, 46H) 1.5-1.7 (m, 12H) 2.3 (t, J=7.6 Hz, 4H) 2.4 (t, J=7.4 Hz, 4H) 4.1 (t, J=7.1 Hz, 4H).

Step d):

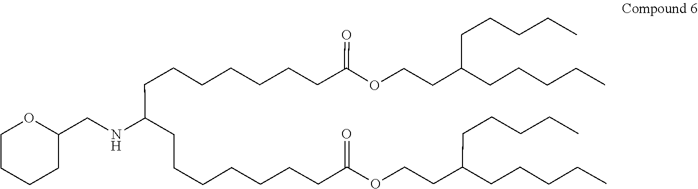 [SMILES: CCCCCC(CCCCC)CCOC(=O)CCCCCCCC(CCCCCCCC(=O)OCCC(CCCCC)CCCCC)NCC1CCCCO1]

sodium triacetoxyhydroborate (32.1 mg, 0.15 mmol) was added in one portion to a stirred solution of (tetrahydro-2H-pyran-2-yl)methanaminium chloride (20.07 mg, 0.13 mmol) and bis(3-pentyloctyl) 9-oxoheptadecanedioate (42.8 mg, 0.06 mmol) in 1,2-DCE (2 mL) and NMP (0.5 mL) at 25° C. under argon. The resulting solution was stirred at 25° C. for 30 hours. The reaction mixture was diluted with DCM (15 mL) and water (5 mL) with sat. Na₂CO₃ (10 mL). The layers were separated, and the aqueous layer was extracted with (DCM) (3×15 mL). The combined organic layers were layer was dried over MgSO₄, filtered and concentrated under reduced pressure to dryness to afford crude product. The resulting residue was purified by flash silica chromatography, elution gradient 0 to 30% of 20% MeOH/DCM (w/1% NH₄OH) in DCM. Product fractions were concentrated under reduced pressure to dryness to afford Compound 6 (17.40 mg, 35.5%) as a colorless oil. ¹H NMR (500 MHz, Methanol-d4) δ ppm 0.9 (t, J=6.9 Hz, 12H) 1.2-1.4 (m, 51H) 1.4-1.5 (m, 6H) 1.5-1.6 (m, 12H) 1.9 (br s, 1H) 2.3 (t, J=7.3 Hz, 4H) 2.5-2.6 (m, 2H) 2.7-2.7 (m, 1H) 3.5 (br t, J=9.4 Hz, 2H) 4.0 (br d, J=11.1 Hz, 1H) 4.1-4.1 (m, 4H); C₄₉H₉₅NO₅ m/z calcd. 777.721 observed 778.9 [M+H]⁺ (LCMS).

### Example 7. Synthesis of Compound 7: bis(3-pentyloctyl) 9-((2-(tetrahydro-2H-pyran-4-yl)ethyl)amino)heptadecanedioate

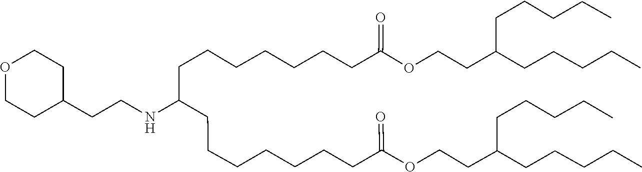 [SMILES: CCCCCC(CCCCC)CCOC(=O)CCCCCCCC(CCCCCCCC(=O)OCCC(CCCCC)CCCCC)NCCC1CCOCC1]

Compound 7 was prepared according to the protocol described for Compound 6 using tetraethyl 8-oxopentadecane-1,7,9,15-tetracarboxylate, 9-oxoheptadecanedioic acid and bis(3-pentyloctyl) 9-oxoheptadecanedioate with the following additional steps. Sodium triacetoxyhydroborate (43.7 mg, 0.21 mmol) was added in one portion to a stirred solution of 2-(tetrahydro-2H-pyran-4-yl)ethan-1-amine (0.026 mL, 0.19 mmol), acetic acid (0.012 mL, 0.21 mmol) and bis(3-pentyloctyl) 9-oxoheptadecanedioate (46.7 mg, 0.07 mmol) in DCE (2 mL) and NMP (0.5 mL) at 25° C. under argon. The resulting solution was stirred at 25° C. for 30 hours. The reaction mixture was diluted with DCM (15 mL) and water (5 mL) with sat. Na₂CO₃ (10 mL). The layers were separated, and the aqueous layer was extracted with (DCM) (3×15 mL). The combined organic layers were layer was dried over MgSO₄, filtered and concentrated under reduced pressure to dryness to afford crude product. The resulting residue was purified by flash silica chromatography, elution gradient 0 to 30% of 20% MeOH/DCM (w/1% NH₄OH) in DCM. Product fractions were concentrated under reduced pressure to dryness to afford Compound 7 (23.20 mg, 42.6%) as a colorless oil. ¹H NMR (400 MHz, Methanol-d4) δ ppm 0.9 (t, J=7.0 Hz, 12H) 1.3 (br s, 50H) 1.4-1.5 (m, 8H) 1.6 (br d, J=6.6 Hz, 11H) 2.3 (s, 4H) 2.5-2.6 (m, 1H) 2.6-2.7 (m, 2H) 3.4-3.5 (m, 2H) 3.9-4.0 (m, 2H) 4.1-4.1 (m, 4H); C₅₀H₉₇NO₅ m/z calcd. 791.737 observed 792.8 [M+H]⁺ (LCMS).

### Example 8. Synthesis of Compound 8: bis(3-pentyloctyl) 9-(((tetrahydrofuran-2-yl)methyl)amino)heptadecanedioate

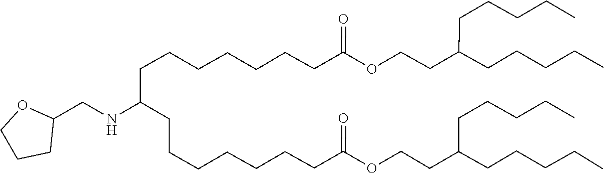 [SMILES: CCCCCC(CCCCC)CCOC(=O)CCCCCCCC(CCCCCCCC(=O)OCCC(CCCCC)CCCCC)NCC1CCCO1]

Compound 8 was prepared according to the protocol described for Compound 6 using tetraethyl 8-oxopentadecane-1,7,9,15-tetracarboxylate, 9-oxoheptadecanedioic acid and bis(3-pentyloctyl) 9-oxoheptadecanedioate with the following additional steps. Sodium triacetoxyhydroborate (43.0 mg, 0.20 mmol) was added in one portion to a stirred solution of (tetrahydrofuran-2-yl)methanamine (0.020 mL, 0.20 mmol), acetic acid (0.012 mL, 0.22 mmol) and bis(3-pentyloctyl) 9-oxoheptadecanedioate (49.2 mg, 0.07 mmol) in 1,2-DCE (2 mL) and NMP (0.5 mL) at 25° C. under argon. The resulting solution was stirred at 25° C. for 30 hours. The reaction mixture was diluted with DCM (15 mL) and water (5 mL) with sat. Na₂CO₃ (10 mL). The layers were separated, and the aqueous layer was extracted with DCM (3×15 mL). The combined organic layers were layer was dried over MgSO₄, filtered and concentrated under reduced pressure to dryness to afford crude product. The resulting residue was purified by flash silica chromatography, elution gradient 0 to 35% of 20% MeOH/DCM (w/1% NH₄OH) in DCM. Product fractions were concentrated under reduced pressure to dryness to afford Compound 9 (22.60 mg, 40.8%) as a colorless oil. ¹H NMR (500 MHz, Methanol-d4) δ ppm 0.9 (t, J=6.9 Hz, 12H) 1.2-1.4 (m, 49H) 1.4-1.5 (m, 6H) 1.5-1.7 (m, 9H) 1.9-2.0 (m, 2H) 2.0-2.1 (m, 1H) 2.3 (s, 4H) 2.5-2.6 (m, 2H) 2.7-2.8 (m, 1H) 3.8 (br d, J=7.3 Hz, 1H) 3.8 (br d, J=7.6 Hz, 1H) 4.0 (br d, J=5.6 Hz, 1H) 4.1-4.1 (m, 4H); C₄₈H₉₃NO₅ m/z calcd. 763.705 observed 764.9 [M+H]⁺ (LCMS).

### Example 9. Synthesis of Compound 9: bis(3-pentyloctyl) 9-(((1,4-dioxan-2-yl)methyl)amino)heptadecanedioate

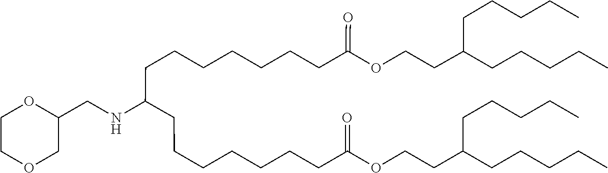 [SMILES: CCCCCC(CCCCC)CCOC(=O)CCCCCCCC(CCCCCCCC(=O)OCCC(CCCCC)CCCCC)NCC1COCCO1]

Compound 9 was prepared according to the protocol described for Compound 6 using tetraethyl 8-oxopentadecane-1,7,9,15-tetracarboxylate, 9-oxoheptadecanedioic acid and bis(3-pentyloctyl) 9-oxoheptadecanedioate with the following additional steps. Sodium triacetoxyhydroborate (36.6 mg, 0.17 mmol) was added in one portion to a stirred solution of (1,4-dioxan-2-yl)methanamine (0.017 mL, 0.16 mmol), acetic acid (9.88 μl, 0.17 mmol) and bis(3-pentyloctyl) 9-oxoheptadecanedioate (39.1 mg, 0.06 mmol) in 1,2-DCE (2 mL) and NMP (0.5 mL) at 25° C. under argon. The resulting solution was stirred at 25° C. for 30 hours. The reaction mixture was diluted with DCM (15 mL) and water (5 mL) with sat. Na₂CO₃ (10 mL). The layers were separated, and the aqueous layer was extracted with DCM (3×15 mL). The combined organic layers were layer was dried over MgSO₄, filtered and concentrated under reduced pressure to dryness to afford crude product. The resulting residue was purified by flash silica chromatography, elution gradient 0 to 35% of 20% MeOH/DCM (w/1% NH₄OH) in DCM. Product fractions were concentrated under reduced pressure to dryness to afford Compound 9 (23.10 mg, 51.4%) as a colorless oil. ¹H NMR (500 MHz, Methanol-d4) δ ppm 0.9 (t, J=7.1 Hz, 12H) 1.3-1.4 (m, 48H) 1.4-1.5 (m, 6H) 1.6-1.6 (m, 8H) 2.3 (t, J=7.3 Hz, 4H) 2.5-2.6 (m, 2H) 2.6 (s, 3H) 3.3-3.3 (m, 1H) 3.6 (s, 1H) 3.6-3.8 (m, 4H) 3.8-3.8 (m, 1H) 4.1 (s, 4H); C₄₈H₉₃NO₆ m/z calcd. 779.700 observed 780.9 [M+H]⁺ (LCMS).

Scheme 3 below illustrates the synthetic procedures for preparing Examples 10 to 12. In step g below, AcOH as an additive was used in reductive amination when utilizing the respective amines as a free base.

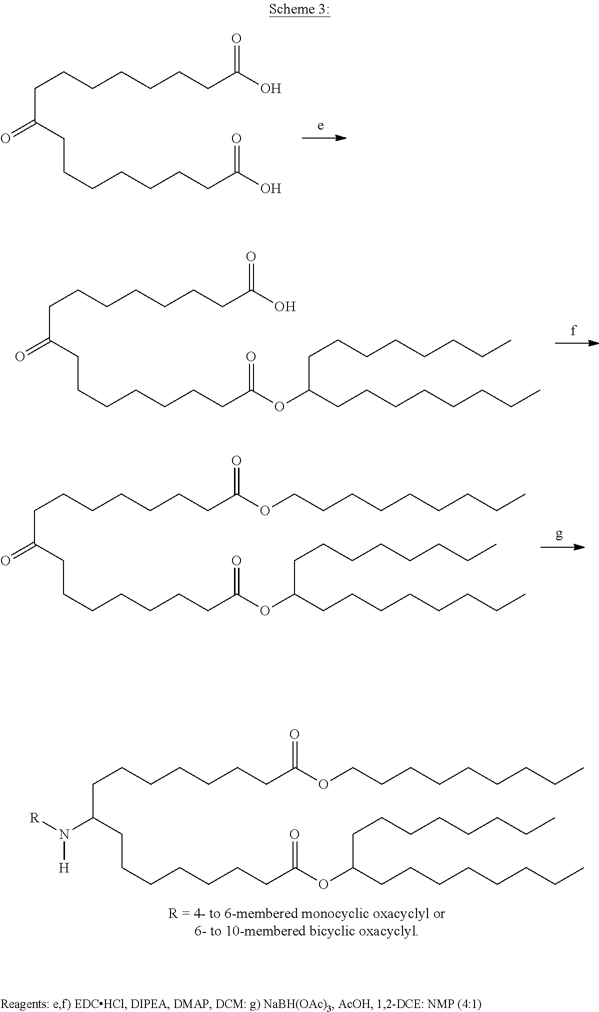

### Example 10. Synthesis of Compound 10: 1-(heptadecan-9-yl) 17-nonyl 9-((2-oxaspiro[3.3]heptan-6-yl)amino)heptadecanedioate

Step e): 3-(((ethylimino)methylene)amino)-N,N-dimethylpropan-1-amine hydrochloride (97 mg, 0.50 mmol) was added in one portion to a stirred solution of 9-oxoheptadecanedioic acid (105.6 mg, 0.34 mmol), N-ethyl-N-isopropylpropan-2-amine (176 μl, 1.01 mmol), heptadecan-9-ol (57.5 mg, 0.22 mmol) and N,N-dimethylpyridin-4-amine (8.62 mg, 0.07 mmol) in DCM (6 mL) at 0° C. under argon. The resulting solution was stirred at 25° C. for 18 hours. The reaction mixture was concentrated under reduced pressure to dryness and redissolved in EtOAc (15 mL), water (5 mL) and 5% citric acid (10 mL). The layers were separated, and the aqueous layer was extracted three times with EtOAc (20 mL). The combined organic layers were dried over MgSO₄, filtered and concentrated under reduced pressure to dryness to afford crude product. The resulting residue was purified by flash silica chromatography, elution gradient 10 to 40% EtOAc in hexanes. Product fractions were concentrated under reduced pressure to dryness to afford 17-(heptadecan-9-yloxy)-9,17-dioxoheptadecanoic acid (0.083 g, 44.4%) as a colorless oil. ¹H NMR (500 MHz, Chloroform-d) δ ppm 0.8-0.9 (m, 6H) 1.2-1.3 (m, 36H) 1.5-1.6 (m, 8H) 1.6-1.6 (m, 4H) 2.2-2.4 (m, 8H) 4.8-4.9 (m, 1H).

Step f): 3-(((ethylimino)methylene)amino)-N,N-dimethylpropan-1-amine hydrochloride (145 mg, 0.76 mmol) was added in one portion to a stirred solution of nonan-1-ol (124 μl, 0.71 mmol), N,N-dimethylpyridin-4-amine (6.08 mg, 0.05 mmol), 17-(heptadecan-9-yloxy)-9,17-dioxoheptadecanoic acid (131 mg, 0.24 mmol) and N-ethyl-N-isopropylpropan-2-amine (174 μl, 1.00 mmol) in DCM (4.5 mL) at 0° C. under argon. The resulting solution was stirred at 25° C. for 18 hours. The reaction mixture was diluted with DCM (10 mL) and sat. NH₄Cl (10 mL). The layers were separated, and the aqueous layer was extracted three times with DCM (15 mL). The combined organic layers were MgSO₄, filtered and concentrated under reduced pressure to dryness to afford crude product. The resulting residue was purified by flash silica chromatography, elution gradient 10 to 30% EtOAc in hexanes. Product fractions were concentrated under reduced pressure to afford 1-(heptadecan-9-yl) 17-nonyl 9-oxoheptadecanedioate (0.135 g, 84%) as a colorless oil. ¹H NMR (500 MHz, Chloroform-d) δ ppm 0.9-0.9 (m, 9H) 1.2-1.3 (m, 26H) 1.3 (br s, 22H) 1.5-1.7 (m, 14H) 2.3 (q, J=7.3 Hz, 4H) 2.4 (t, J=7.5 Hz, 4H) 4.1 (t, J=6.8 Hz, 2H) 4.8-4.9 (m, 1H).

Step g):

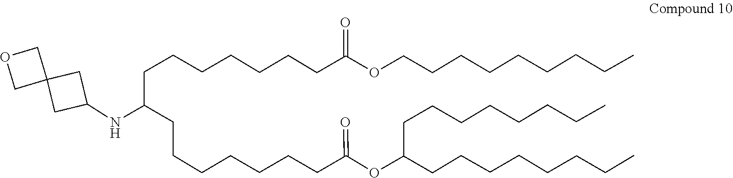 [SMILES: CCCCCCCCCOC(=O)CCCCCCCC(CCCCCCCC(=O)OC(CCCCCCCC)CCCCCCCC)NC1CC2(COC2)C1]

sodium triacetoxyhydroborate (35.3 mg, 0.17 mmol) was added in one portion to a stirred solution of 1-(heptadecan-9-yl) 17-nonyl 9-oxoheptadecanedioate (45.3 mg, 0.07 mmol), and 2-oxaspiro[3.3]heptan-6-aminium chloride (23.95 mg, 0.16 mmol) in 1,2-DCE (2 mL) and NMP (0.5 mL) under argon. The resulting solution was stirred at 25° C. for 50 hours. The reaction mixture was diluted with DCM (10 mL), water (5 mL) and sat. Na₂CO₃ (5 mL). The layers were separated, and the aqueous layer was extracted three times with DCM (15 mL). The combined organic layers were dried over MgSO₄, filtered and concentrated under reduced pressure to dryness to afford crude product. The resulting residue was purified by flash silica chromatography, elution gradient 0 to 50% of 20% MeOH/DCM (w/1% NH₄OH) in DCM. Product fractions were concentrated under reduced pressure to dryness to afford Compound 10 (0.039 g, 75%) as a colorless oil. ¹H NMR (500 MHz, Methanol-d4) δ ppm 0.9 (br t, J=6.5 Hz, 9H) 1.3-1.4 (m, 56H) 1.5-1.6 (m, 4H) 1.6 (br d, J=6.1 Hz, 6H) 2.0 (br t, J=10.2 Hz, 2H) 2.3 (br t, J=7.2 Hz, 4H) 2.5-2.6 (m, 3H) 3.2 (br t, J=7.8 Hz, 1H) 4.1 (t, J=6.5 Hz, 2H) 4.6 (s, 2H) 4.7 (s, 2H) 4.9-4.9 (m, 1H); C₄₉H₉₃NO₅ m/z calcd. 775.705 observed 776.8 [M+H]⁺ (LCMS).

### Example 11. Synthesis of Compound 11: 1-(heptadecan-9-yl) 17-nonyl 9-((7-oxaspiro[3.5]nonan-2-yl)amino)heptadecanedioate

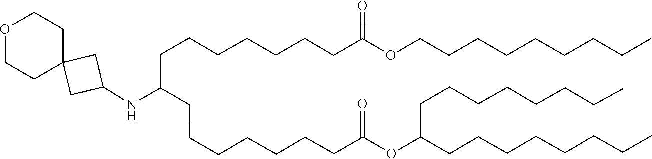 [SMILES: CCCCCCCCCOC(=O)CCCCCCCC(CCCCCCCC(=O)OC(CCCCCCCC)CCCCCCCC)NC1CC2(CCOCC2)C1]

Compound 11 was prepared according to the protocol described for Compound 10 using 17-(heptadecan-9-yloxy)-9,17-dioxoheptadecanoic acid and 1-(heptadecan-9-yl) 17-nonyl 9-oxoheptadecanedioate with the following additional steps. Sodium triacetoxyhydroborate (34.3 mg, 0.16 mmol) was added in one portion to a stirred solution of 1-(heptadecan-9-yl) 17-nonyl 9-oxoheptadecanedioate (43.9 mg, 0.06 mmol), and 7-oxaspiro[3.5]nonan-2-aminium chloride (27.6 mg, 0.16 mmol) in 1,2-DCE (2 mL) and NMP (0.5 mL) under argon. The resulting solution was stirred at 25° C. for 50 hours. The reaction mixture was diluted with DCM (10 mL), water (5 mL) and sat. Na₂CO₃ (5 mL).

The layers were separated, and the aqueous layer was extracted three times with DCM (15 mL). The combined organic layers were dried over MgSO₄, filtered and concentrated under reduced pressure to dryness to afford crude product. The resulting residue was purified by flash silica chromatography, elution gradient 0 to 50% of 20% MeOH/DCM (w/1% NH₄OH) in DCM. Product fractions were concentrated under reduced pressure to dryness to afford Compound 11 (0.029 g, 55.4%) as a colorless oil. ¹H NMR (500 MHz, Methanol-d4) δ ppm 0.9 (t, J=6.8 Hz, 9H) 1.3-1.4 (m, 56H) 1.5-1.6 (m, 8H) 1.6 (br d, J=5.6 Hz, 8H) 2.2-2.3 (m, 2H) 2.3-2.3 (m, 4H) 2.5 (br t, J=5.4 Hz, 1H) 3.3-3.4 (m, 1H) 3.5-3.6 (m, 2H) 3.6-3.7 (m, 2H) 4.1 (s, 2H) 4.9-4.9 (m, 1H); C₅₁H₉₇NO₅ m/z calcd. 803.737 observed 804.7 [M+H]+(LCMS).

### Example 12. Synthesis of Compound 12: 1-(heptadecan-9-yl) 17-nonyl 9-((tetrahydro-2H-pyran-4-yl)amino)heptadecanedioate

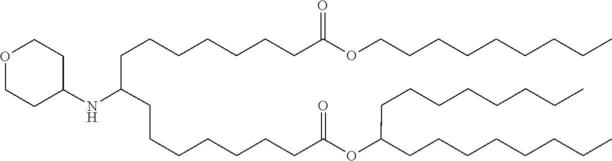 [SMILES: CCCCCCCCCOC(=O)CCCCCCCC(CCCCCCCC(=O)OC(CCCCCCCC)CCCCCCCC)NC1CCOCC1]

Compound 12 was prepared according to the protocol described for Compound 10 using 17-(heptadecan-9-yloxy)-9,17-dioxoheptadecanoic acid and 1-(heptadecan-9-yl) 17-nonyl 9-oxoheptadecanedioate with the following additional steps. Sodium triacetoxyhydroborate (30.2 mg, 0.14 mmol) was added in one portion to a stirred solution of tetrahydro-2H-pyran-4-amine (12.65 μl, 0.12 mmol), 1-(heptadecan-9-yl) 17-nonyl 9-oxoheptadecanedioate (46.1 mg, 0.07 mmol) and acetic acid (163 μl, 0.16 mmol) in 1,2-DCE (2 mL) and NMP (0.5 mL) under argon. The resulting solution was stirred at 25° C. for 40 hours. The reaction mixture was diluted with DCM (10 mL), water (5 mL) and sat. Na₂CO₃ (5 mL). The layers were separated, and the aqueous layer was extracted three times with DCM (15 mL). The combined organic layers were dried over MgSO₄, filtered and concentrated under reduced pressure to dryness to afford crude product. The resulting residue was purified by flash silica chromatography, elution gradient 0 to 45% of 20% MeOH/DCM (w/1% NH₄OH) in DCM. Product fractions were concentrated under reduced pressure to dryness to afford Compound 12 (0.028 g, 53.8%) as a colorless oil. ¹H NMR (500 MHz, Methanol-d4) δ ppm 0.9 (br t, J=6.3 Hz, 9H) 1.3-1.3 (m, 27H) 1.3-1.5 (m, 31H) 1.5-1.6 (m, 4H) 1.6-1.7 (m, 6H) 1.8 (br d, J=12.5 Hz, 2H) 2.3 (s, 4H) 2.7-2.7 (m, 1H) 2.8 (brt, J=10.5 Hz, 1H) 3.4 (br t, J=11.7 Hz, 2H) 3.9 (br d, J=10.8 Hz, 2H) 4.0-4.1 (m, 2H) 4.9-4.9 (m, 1H); C₄₈H₉₃NO₅ m/z calcd. 763.705 observed 764.8 [M+H]⁺ (LCMS).

Scheme 4 below illustrates the synthetic procedures for preparing Examples 13 and 45. In step k below, AcOH as an additive was used in reductive amination when utilizing the respective amines as a free base.

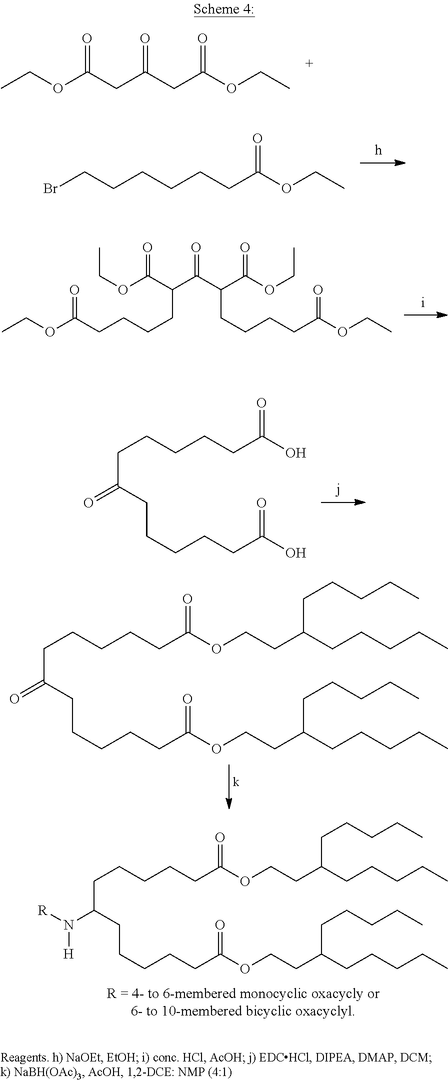

### Example 13. Synthesis of Compound 13: bis(3-pentyloctyl) 7-((2-oxaspiro[3.3]heptan-6-yl)amino)tridecanedioate

Step h): sodium ethanolate (1.111 g, 16.32 mmol) was added portionwise to a stirred solution of diethyl 3-oxopentanedioate (1.977 ml, 10.88 mmol) in ethanol (absolute, 99.5%) (10 mL) at 81° C. under argon. The resulting solution was stirred at 81° C. for 1 hour. To it was added ethyl 5-bromopentanoate (3.87 ml, 24.48 mmol) dropwise and stirred at 81 degree C. for 18 hours. The reaction mixture was cooled down, solvent was evaporated and reaction mixture was diluted with DCM (25 mL), and washed with saturated aqueous NaCl (25 mL). The organic layer was dried over MgSO₄, filtered and concentrated under reduced pressure to dryness to afford crude product. The resulting residue was purified by flash silica chromatography, elution gradient 0 to 60% EtOAc in hexanes. Product fractions were concentrated under reduced pressure to afford tetraethyl 6-oxoundecane-1,5,7,11-tetracarboxylate (3.28 g, 65.8%) as a pale yellow liquid. ¹H NMR (500 MHz, Chloroform-d) δ ppm 1.2-1.4 (m, 16H) 1.6-1.7 (m, 4H) 1.8-2.0 (m, 4H) 2.3-2.4 (m, 4H) 4.1-4.3 (m, 10H).

Step i): hydrogen chloride (17.65 ml, 214.99 mmol) was added slowly to a stirred solution of tetraethyl 6-oxoundecane-1,5,7,11-tetracarboxylate (3.821 g, 8.33 mmol) in acetic acid (8.5 mL) at 25° C. The resulting solution was stirred at 102° C. for 18 hours w/reflux condenser and a vent to get rid of excess of HCl gas. Reaction was cooled down to RT and poured the reaction mixture on ice-water (50 mL) and was allowed to sit for 30 min. The precipitate was collected by filtration, washed with cold water (3×20 mL) and dried under vacuum to afford crude material as a yellow solid. The crude product was purified by crystallization from acetone to afford 7-oxotridecanedioic acid (0.283 g, 13.15%) as a white powder. ¹H NMR (500 MHz, DMSO-d6) δ ppm 1.2-1.2 (m, 4H) 1.4 (m, 8H) 2.2 (t, J=7.3 Hz, 4H) 2.4 (t, J=7.3 Hz, 4H); C₁₃H₂₂O₅ m/z calcd. 258.147 observed 257.1 [M−H]⁻ (LCMS).

Step j): 3-(((ethylimino)methylene)amino)-N,N-dimethylpropan-1-amine hydrochloride (204 mg, 1.06 mmol) was added in one portion to a stirred solution of 7-oxotridecanedioic acid (101.8 mg, 0.39 mmol), 3-pentyloctan-1-ol (see WO 2013/086354, p191 for the procedures of preparing 3-pentyloctan-1-ol) (197 mg, 0.99 mmol), N-ethyl-N-isopropylpropan-2-amine (275 μl, 1.58 mmol) and N,N-dimethylpyridin-4-amine (9.63 mg, 0.08 mmol) in DCM (7 mL) at 0° C. under argon. The resulting solution was stirred at 25° C. for 24 hours. The reaction mixture was quenched with saturated aqueous NH₄Cl (15 mL), extracted with EtOAc (3×20 mL), the organic layer was dried over MgSO₄, filtered and concentrated under reduced pressure to dryness to afford colorless oil. The resulting residue was purified by flash silica chromatography, elution gradient 0 to 40% EtOAc in hexanes. Product fractions were concentrated under reduced pressure to dryness to afford bis(3-pentyloctyl) 7-oxotridecanedioate (0.227 g, 92%) as a colorless oil. ¹H NMR (500 MHz, Chloroform-d) δ ppm 0.9 (t, J=7.1 Hz, 12H) 1.2-1.4 (m, 36H) 1.4 (br d, J=5.0 Hz, 2H) 1.5-1.7 (m, 12H) 2.3 (t, J=7.5 Hz, 4H) 2.4 (t, J=7.5 Hz, 4H) 4.1 (t, J=7.1 Hz, 4H).

Step k):

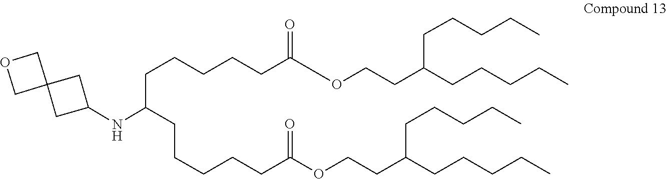 [SMILES: CCCCCC(CCCCC)CCOC(=O)CCCCCC(CCCCCC(=O)OCCC(CCCCC)CCCCC)NC1CC2(COC2)C1]

sodium triacetoxyhydroborate (44.2 mg, 0.21 mmol) was added in one portion to a stirred solution of bis(3-pentyloctyl) 7-oxotridecanedioate (52 mg, 0.08 mmol), and 2-oxaspiro[3.3]heptan-6-aminium chloride (30.0 mg, 0.20 mmol) in NMP (0.500 mL) and 1,2-DCE (2 mL) at 25° C. under argon. The resulting suspension was stirred at 25° C. for 40 hours. The reaction mixture was diluted with DCM (15 mL), water (5 mL) and sat. Na₂CO₃ (10 mL). The layers were separated, and the aqueous layer was extracted with DCM (3×15 mL). The combined organic layers were dried over MgSO₄, filtered and concentrated under reduced pressure to dryness to afford crude product. The resulting residue was purified by flash silica chromatography, elution gradient 0 to 50% of 20% MeOH/DCM (w/1% NH₄OH) in DCM. Product fractions were concentrated under reduced pressure to dryness to afford Compound 13 (0.025 g, 41.8%) as a colorless oil. ¹H NMR (500 MHz, Methanol-d4) δ ppm 0.9-1.0 (m, 12H) 1.3-1.4 (m, 44H) 1.4-1.5 (m, 2H) 1.5-1.7 (m, 8H) 1.9-2.0 (m, 2H) 2.3 (t, J=7.3 Hz, 4H) 2.4-2.6 (m, 3H) 3.2 (t, J=7.7 Hz, 1H) 4.1 (t, J=6.8 Hz, 4H) 4.6 (s, 2H) 4.7 (s, 2H); C₄₈H₉₃NO₅ m/z calcd. 719.643 observed 720.7 [M+H]⁺ (LCMS).

Scheme 5 below illustrates the synthetic procedures for preparing Examples 14-18. In step o blow, AcOH as an additive was used in reductive amination when utilizing the respective amines as a free base.

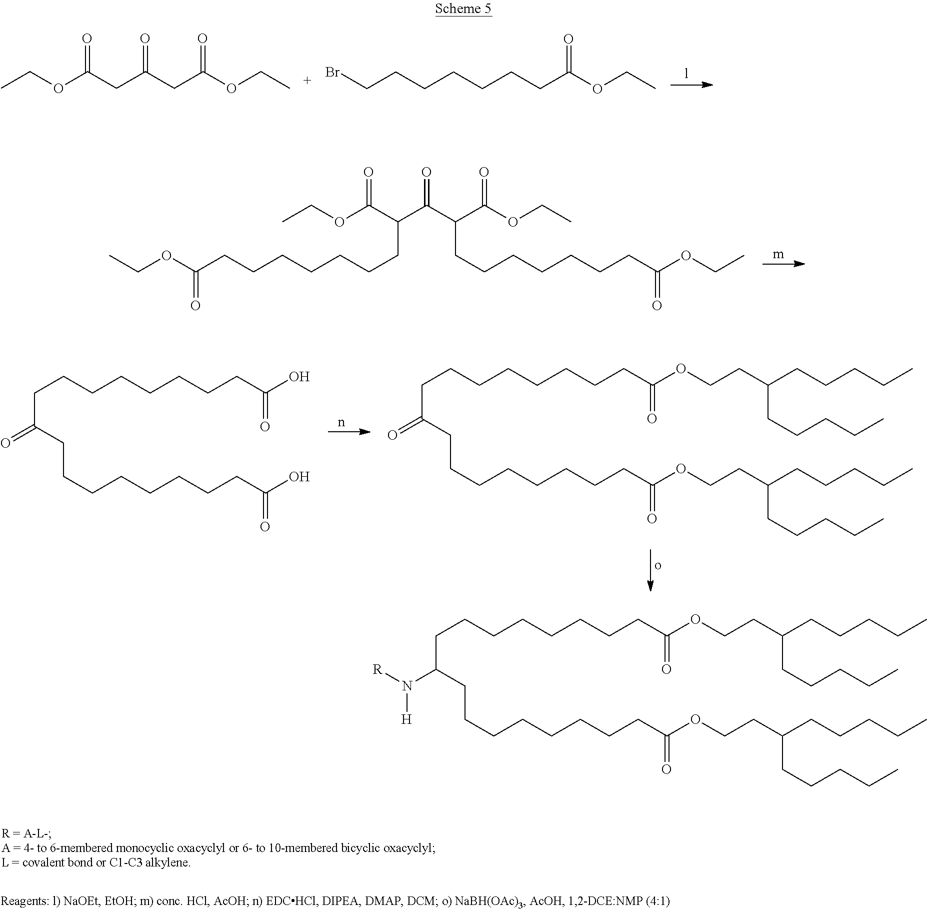

### Example 14. Synthesis of Compound 14: bis(3-pentyloctyl) 10-((2-oxaspiro[3.3]heptan-6-yl)amino)nonadecanedioate

Step l): sodium ethanolate (2.52 g, 37.09 mmol) was added portionwise to a stirred solution of diethyl 3-oxopentanedioate (4.49 ml, 24.73 mmol) in ethanol (absolute, 99.5%) (15 mL) at 25 C over a period of 10 minutes under argon. The resulting suspension was stirred at 81° C. for 1 hour. To the reaction mixture ethyl 8-bromooctanoate (12.53 ml, 59.35 mmol) was added dropwise and the suspension was stirred at 81° C. for a further 18 hours. The reaction mixture was cooled down to RT and concentrated under reduced pressure to dryness and redissolved in DCM (50 mL), and washed sequentially with water (50 mL), saturated aqueous sodium chloride (50 mL). The organic layer was dried over MgSO₄, filtered and concentrated under reduced pressure to afford crude product. The resulting residue was purified by flash silica chromatography, elution gradient 0 to 50% EtOAc in hexanes. Product fractions were concentrated under reduced pressure to dryness to afford tetraethyl 9-oxoheptadecane-1,8,10,17-tetracarboxylate (8.80 g, 65.6%) as a pale yellow oil. ¹H NMR (500 MHz, Chloroform-d) δ ppm 1.2-1.4 (m, 28H) 1.6-1.7 (m, 4H) 1.7-1.9 (m, 4H) 2.2-2.3 (m, 4H) 4.1-4.2 (m, 10H).

Step m): hydrogen chloride (34.4 ml, 418.34 mmol) was added slowly to a stirred solution of tetraethyl tetraethyl 9-oxoheptadecane-1,8,10,17-tetracarboxylate (8.8 g, 16.21 mmol) in acetic acid (21 mL) at 25° C. The resulting solution was stirred at 102° C. for 18 hours w/reflux condenser and a vent to get rid of excess of HCl gas. Reaction was cooled down to RT and poured the reaction mixture on ice-water (40 mL) and was allowed to sit for 30 min. The precipitate was collected by filtration, washed with cold water (3×20 mL) and dried under vacuum to afford crude product as pale yellow solid. The crude product was purified by crystallization from acetone to afford 10-oxononadecanedioic acid (1.963 g, 35.3%). ¹H NMR (500 MHz, DMSO-d6) δ ppm 1.2 (br s, 16H) 1.4-1.5 (m, 8H) 2.2 (t, J=7.3 Hz, 4H) 2.3-2.4 (m, 4H) 12.0 (br s, 2H); C₁₉H₃₄O₅ m/z calcd. 342.241 observed 341.2 [M−H]⁻ (LCMS).

Step n): 3-(((ethylimino)methylene)amino)-N,N-dimethylpropan-1-amine hydrochloride (272 mg, 1.42 mmol) was added in one portion to a stirred solution of 10-oxononadecanedioic acid (180 mg, 0.53 mmol), N,N-dimethylpyridin-4-amine (9.63 mg, 0.08 mmol), 3-pentyloctan-1-ol (see WO 2013/086354, p191 for the procedures of preparing 3-pentyloctan-1-ol) (253 mg, 1.26 mmol) and N-ethyl-N-isopropylpropan-2-amine (330 μl, 1.89 mmol) in DCM (8 mL) at 0° C. under argon. The resulting solution was stirred at 25° C. for 18 hours. The reaction mixture was diluted with DCM (15 mL) and water (15 mL). The layers were separated, and the aqueous layer was extracted with DCM (3×20 mL). The combined organic layers were washed with 0.5 M citric acid (15 mL) and saturated aqueous NaCl (15 mL). The organic layer was dried over MgSO₄, filtered and concentrated under reduced pressure to dryness to afford crude product. The resulting residue was purified by flash silica chromatography, elution gradient 0 to 30% hexanes in EtOAc. Product fractions were concentrated under reduced pressure to dryness to afford bis(3-pentyloctyl) 10-oxononadecanedioate (0.343 g, 92%) as a colorless oil. ¹H NMR (500 MHz, Chloroform-d) δ ppm 0.9 (t, J=7.1 Hz, 12H) 1.2-1.4 (m, 48H) 1.4 (br s, 2H) 1.5-1.6 (m, 12H) 2.3 (t, J=7.5 Hz, 4H) 2.3-2.4 (m, 4H) 4.0-4.1 (m, 4H).

Step o):

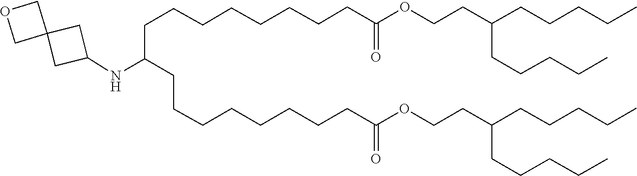 [SMILES: CCCCCC(CCCCC)CCOC(=O)CCCCCCCCC(CCCCCCCCC(=O)OCCC(CCCCC)CCCCC)NC1CC2(COC2)C1]

sodium triacetoxyhydroborate (54.4 mg, 0.26 mmol) was added in one portion to a stirred solution of bis(3-pentyloctyl) 10-oxononadecanedioate (75.7 mg, 0.11 mmol) and 2-oxaspiro[3.3]heptan-6-aminium chloride (33.6 mg, 0.22 mmol) in 1,2-DCE (2.4 mL) and NMP (0.6 mL) at 25° C. under argon. The resulting suspension was stirred at 25° C. for 40 hours. The reaction mixture was diluted with DCM (10 mL), water (5 mL) and sat. Na₂CO₃ (5 mL). The layers were separated, and the aqueous layer was extracted with DCM (3×10 mL). The combined organic layer was dried over MgSO₄, filtered and concentrated under reduced pressure to dryness to afford crude product. The resulting residue was purified by flash silica chromatography, elution gradient 0 to 35% of 20% MeOH/DCM (w/1% NH₄OH) in DCM. Product fractions were concentrated under reduced pressure to dryness to afford Compound 14 (0.033 g, 38.0%) as a colorless oil. ¹H NMR (500 MHz, Methanol-d4) δ ppm 0.9 (t, J=7.0 Hz, 12H) 1.2-1.4 (m, 56H) 1.4-1.5 (m, 2H) 1.5-1.6 (m, 8H) 2.0 (td, J=9.1, 2.8 Hz, 2H) 2.3 (t, J=7.3 Hz, 4H) 2.4-2.6 (m, 3H) 3.2 (br t, J=7.8 Hz, 1H) 4.1 (t, J=6.7 Hz, 4H) 4.6 (s, 2H) 4.7 (s, 1H); C₅₁H₉₇NO₅ m/z calcd. 803.737 observed 804.8 [M+H]⁺ (LCMS).

### Example 15. Synthesis of Compound 15: bis(3-pentyloctyl) 10-((tetrahydro-2H-pyran-4-yl)amino)nonadecanedioate

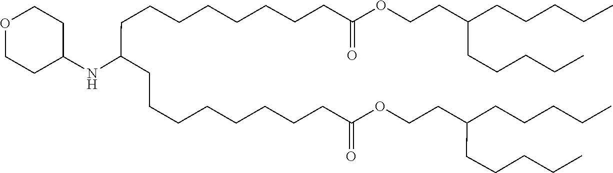 [SMILES: CCCCCC(CCCCC)CCOC(=O)CCCCCCCCC(CCCCCCCCC(=O)OCCC(CCCCC)CCCCC)NC1CCOCC1]

Compound 15 was prepared according to the protocol described for Compound 14 using tetraethyl 9-oxoheptadecane-1,8,10,17-tetracarboxylate, 10-oxononadecanedioic acid and bis(3-pentyloctyl) 10-oxononadecanedioate with the following additional steps. Sodium triacetoxyhydroborate (75 mg, 0.36 mmol) was added in one portion to a stirred solution of bis(3-pentyloctyl) 10-oxononadecanedioate (93 mg, 0.13 mmol), acetic acid (22.56 μl, 0.39 mmol) and tetrahydro-2H-pyran-4-amine (32.7 μl, 0.32 mmol) in 1,2-DCE (2.4 mL) and NMP (0.6 mL) at 25° C. under argon. The resulting suspension was stirred at 25° C. for 50 hours. The reaction mixture was diluted with DCM (10 mL), water (5 mL) and sat. Na₂CO₃ (5 mL). The layers were separated, and the aqueous layer was extracted with DCM (3×10 mL). The combined organic layer was dried over MgSO₄, filtered and concentrated under reduced pressure to dryness to afford crude product. The resulting residue was purified by flash silica chromatography, elution gradient 0 to 35% of 20% MeOH/DCM (w/1% NH₄OH) in DCM. Product fractions were concentrated under reduced pressure to dryness to afford Compound 15 (0.036 g, 34.4%) as a colorless oil. ¹H NMR (500 MHz, Methanol-d4) δ ppm 0.9 (t, J=7.0 Hz, 12H) 1.2-1.3 (m, 32H) 1.3-1.4 (m, 20H) 1.4-1.5 (m, 8H) 1.5-1.7 (m, 8H) 1.9 (br d, J=12.5 Hz, 2H) 2.3 (t, J=7.3 Hz, 4H) 2.8-2.9 (m, 1H) 2.9-3.0 (m, 1H) 3.4 (br t, J=11.6 Hz, 2H) 4.0 (br dd, J=11.4, 3.7 Hz, 2H) 4.1 (s, 4H); C₅₀H₉₇NO₅ m/z calcd. 791.737 observed 792.8 [M+H]⁺ (LCMS).

### Example 16. Synthesis of Compound 16: bis(3-pentyloctyl) 10-(((tetrahydro-2H-pyran-2-yl)methyl)amino)nonadecanedioate

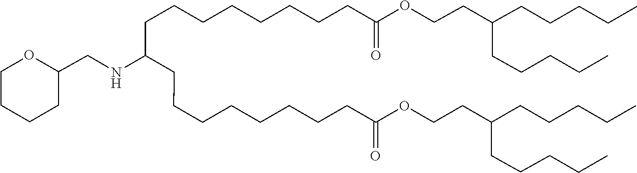 [SMILES: CCCCCC(CCCCC)CCOC(=O)CCCCCCCCC(CCCCCCCCC(=O)OCCC(CCCCC)CCCCC)NCC1CCCCO1]

Compound 16 was prepared according to the protocol described for Compound 14 using tetraethyl 9-oxoheptadecane-1,8,10,17-tetracarboxylate, 10-oxononadecanedioic acid and bis(3-pentyloctyl) 10-oxononadecanedioate with the following additional steps. Sodium triacetoxyhydroborate (41.0 mg, 0.19 mmol) was added in one portion to a stirred solution of bis(3-pentyloctyl) 10-oxononadecanedioate (50.7 mg, 0.07 mmol), and (tetrahydro-2H-pyran-2-yl)methanaminium chloride (27.2 mg, 0.18 mmol) in 1,2-DCE (2.0 mL) and NMP (0.6 mL) at 25° C. under argon. The resulting suspension was stirred at 25° C. for 50 hours. The reaction mixture was diluted with DCM (10 mL), water (5 mL) and sat. Na₂CO₃ (5 mL). The layers were separated, and the aqueous layer was extracted with DCM (5×10 mL). The combined organic layer was dried over MgSO₄, filtered and concentrated under reduced pressure to dryness to afford crude product. The resulting residue was purified by flash silica chromatography, elution gradient 0 to 35% of 20% MeOH/DCM (w/1% NH₄OH) in DCM. Product fractions were concentrated under reduced pressure to dryness to afford Compound 16 (26.9 mg, 46.5%) as a colorless oil. ¹H NMR (400 MHz, Methanol-d4) δ ppm 0.9 (t, J=7.0 Hz, 12H) 1.3 (br d, J=13.8 Hz, 54H) 1.4-1.5 (m, 6H) 1.5-1.7 (m, 12H) 1.8-1.9 (m, 1H) 2.3 (s, 4H) 2.5-2.6 (m, 2H) 2.6-2.7 (m, 1H) 3.4-3.5 (m, 2H) 3.9-4.0 (m, 1H) 4.1-4.1 (m, 4H); C₅₁H₉₉NO₅ m/z calcd. 805.752 observed 807.0 [M+H]⁺ (LCMS).

### Example 17. Synthesis of Compound 17: bis(3-pentyloctyl) 10-(((tetrahydro-2H-pyran-4-yl)methyl)amino)nonadecanedioate

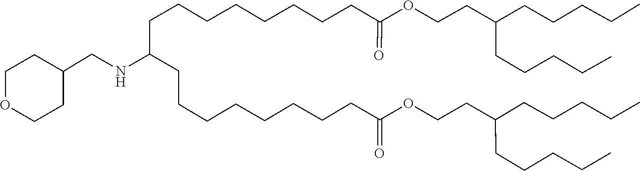 [SMILES: CCCCCC(CCCCC)CCOC(=O)CCCCCCCCC(CCCCCCCCC(=O)OCCC(CCCCC)CCCCC)NCC1CCOCC1]

Compound 17 was prepared according to the protocol described for Compound 14 using tetraethyl 9-oxoheptadecane-1,8,10,17-tetracarboxylate, 10-oxononadecanedioic acid and bis(3-pentyloctyl) 10-oxononadecanedioate with the following additional steps. Sodium triacetoxyhydroborate (43.0 mg, 0.20 mmol) was added in one portion to a stirred solution of bis(3-pentyloctyl) 10-oxononadecanedioate (53.2 mg, 0.08 mmol), acetic acid (12.91 μl, 0.23 mmol) and (tetrahydro-2H-pyran-4-yl)methanamine (17.84 μl, 0.16 mmol) in 1,2-DCE (2 mL) and NMP (0.5 mL) under nitrogen. The resulting solution was stirred at 25° C. for 45 hours. The reaction mixture was diluted with DCM (10 mL), water (5 mL) and sat. Na₂CO₃ (5 mL). The layers were separated, and the aqueous layer was extracted with DCM (3×10 mL). The combined organic layers were dried over MgSO₄, filtered and concentrated under reduced pressure to dryness to afford crude product. The resulting residue was purified by flash silica chromatography, elution gradient 0 to 35% of 20% MeOH/DCM (w/1% NH₄OH) in DCM. Product fractions were concentrated under reduced pressure to dryness to afford Compound 17 (28 mg, 45.7%) as a colorless oil. ¹H NMR (500 MHz, METHANOL-d4) δ ppm 0.9-0.9 (m, 12H) 1.2-1.4 (m, 54H) 1.4-1.5 (m, 6H) 1.6-1.6 (m, 8 H) 1.7-1.8 (m, 3H) 2.3 (t, J=7.3 Hz, 4H) 2.5-2.6 (m, 3H) 3.4-3.5 (m, 2H) 3.9 (br dd, J=11.2, 4.0 Hz, 2H) 4.1 (t, J=6.7 Hz, 4H); C₅₁H₉₉NO₅ m/z calcd. 805.752 observed 806.7 [M+H]⁺ (LCMS).

### Example 18. Synthesis of Compound 18: bis(3-pentyloctyl) 10-((oxetan-3-ylmethyl)amino)nonadecanedioate

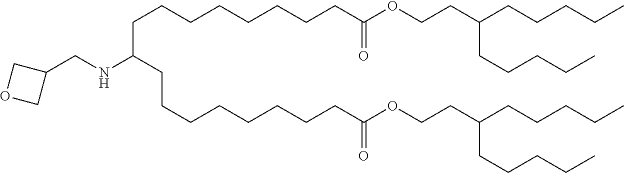 [SMILES: CCCCCC(CCCCC)CCOC(=O)CCCCCCCCC(CCCCCCCCC(=O)OCCC(CCCCC)CCCCC)NCC1COC1]

Compound 18 was prepared according to the protocol described for Compound 14 using tetraethyl 9-oxoheptadecane-1,8,10,17-tetracarboxylate, 10-oxononadecanedioic acid and bis(3-pentyloctyl) 10-oxononadecanedioate with the following additional steps. Sodium triacetoxyhydroborate (31.6 mg, 0.15 mmol) was added in one portion to a stirred solution of oxetan-3-ylmethanaminium chloride (16.40 mg, 0.13 mmol), and bis(3-pentyloctyl) 10-oxononadecanedioate (39.1 mg, 0.06 mmol) in 1,2-DCE (2 mL) and NMP (0.5 mL) at 25° C. under nitrogen. The resulting solution was stirred at 25° C. for 40 hours. The reaction mixture was diluted with DCM (10 mL), water (5 mL) and sat. Na₂CO₃ (5 mL). The layers were separated, and the aqueous layer was extracted with DCM (3×10 mL). The combined organic layers were dried over MgSO₄, filtered and concentrated under reduced pressure to dryness to afford crude product. The resulting residue was purified by flash silica chromatography, elution gradient 0 to 50% of 20% MeOH/DCM (w/1% NH₄OH) in DCM. Product fractions were concentrated under reduced pressure to dryness to afford Compound 18 (0.017 g, 40.4%) as a colorless oil. ¹H NMR (500 MHz, Methanol-d4) δ ppm 0.9 (t, J=6.9 Hz, 12H) 1.3 (br d, J=18.2 Hz, 52H) 1.4-1.5 (m, 6H) 1.5-1.7 (m, 8H) 2.3 (t, J=7.2 Hz, 4H) 2.5 (br t, J=5.7 Hz, 1H) 2.9 (d, J=7.3 Hz, 2H) 3.1-3.2 (m, 1H) 4.1 (t, J=6.6 Hz, 4H) 4.4 (t, J=6.0 Hz, 2H) 4.8 (t, J=6.9 Hz, 2H); C₄₉H₉₅NO₅ m/z calcd. 777.721 observed 778.8 [M+H]⁺ (LCMS).

Scheme 6 below illustrates the synthetic procedures for preparing Examples 19 and 20. In step r below, AcOH as an additive was used in reductive amination when utilizing the respective amines as a free base.

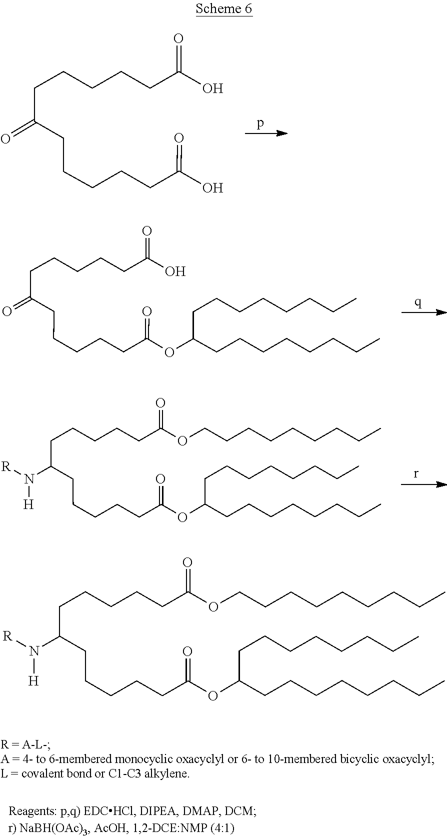

### Example 19. Synthesis of Compound 19: 1-(heptadecan-9-yl) 13-nonyl 7-((2-oxaspiro[3.3]heptan-6-yl)amino)tridecanedioate

Step p): 3-(((ethylimino)methylene)amino)-N,N-dimethylpropan-1-amine hydrochloride (202 mg, 1.05 mmol) was added in one portion to a stirred solution of 7-oxotridecanedioic acid (181.4 mg, 0.70 mmol), N-ethyl-N-isopropylpropan-2-amine (367 μl, 2.11 mmol), heptadecan-9-ol (120 mg, 0.47 mmol) and N,N-dimethylpyridin-4-amine (18.02 mg, 0.15 mmol) in DCM (9 mL) at 0° C. under argon. The resulting solution was stirred at 25° C. for 18 hours. The reaction mixture was concentrated under reduced pressure to dryness and redissolved in EtOAc (15 mL), water (10 mL) and 5% citric acid solution (5 mL). The layers were separated, and the aqueous layer was extracted with EtOAc (3×20 mL). The combined organic layers were dried over MgSO₄, filtered and concentrated under reduced pressure to dryness to afford crude product. The resulting residue was purified by flash silica chromatography, elution gradient 0 to 40% EtOAc in hexanes. Product fractions were concentrated under reduced pressure to dryness to afford 13-(heptadecan-9-yloxy)-7,13-dioxotridecanoic acid (0.102 g, 29.2%) as a colorless oil. ¹H NMR (500 MHz, Chloroform-d) δ ppm 0.9 (t, J=6.9 Hz, 6H) 1.2-1.3 (m, 21H) 1.3-1.4 (m, 7H) 1.5-1.6 (m, 4H) 1.6-1.7 (m, 8H) 2.3 (t, J=7.5 Hz, 2H) 2.3-2.5 (m, 6H) 4.8-4.9 (m, 1H); C₃₀H₅₆O₅ m/z calcd. 496.413 observed 495.5 [M−H]−+ (LCMS).

Step q): 3-(((ethylimino)methylene)amino)-N,N-dimethylpropan-1-amine hydrochloride (83 mg, 0.43 mmol) was added in one portion to a stirred solution of 13-(heptadecan-9-yloxy)-7,13-dioxotridecanoic acid (102 mg, 0.21 mmol), nonan-1-ol (64.2 μl, 0.37 mmol), N,N-dimethylpyridin-4-amine (5.02 mg, 0.04 mmol) and N-ethyl-N-isopropylpropan-2-amine (118 μl, 0.68 mmol) in DCM (6 mL) at 0° C. under nitrogen. The resulting solution was allowed to come to RT stirred at 25° C. for 30 hours. The reaction mixture was diluted with DCM (15 mL) and water (15 mL). The layers were separated, and the aqueous layer was extracted with DCM (3×15 mL). The combined organic layers was washed with 5% citric acid solution (10 mL). The organic layer was dried over MgSO₄, filtered and concentrated under reduced pressure to dryness to afford crude product. The resulting residue was purified by flash silica chromatography, elution gradient 0 to 40% EtOAc in hexanes. Product fractions were concentrated under reduced pressure to dryness to afford 1-(heptadecan-9-yl) 13-nonyl 7-oxotridecanedioate (0.085 g, 66.5%) as a colorless oil. ¹H NMR (500 MHz, Chloroform-d) δ ppm 0.9 (t, J=6.9 Hz, 9H) 1.2-1.4 (m, 40H) 1.5-1.6 (m, 4H) 1.6-1.7 (m, 10H) 2.3 (q, J=7.1 Hz, 4H) 2.4 (t, J=7.4 Hz, 4H) 4.1 (t, J=6.7 Hz, 2H) 4.8-4.9 (m, 1H).

Step r):

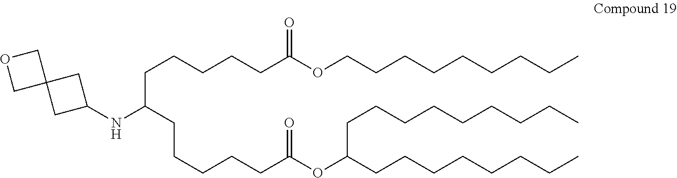 [SMILES: CCCCCCCCCOC(=O)CCCCCC(CCCCCC(=O)OC(CCCCCCCC)CCCCCCCCC)NC1CC2(COC2)C1]

sodium triacetoxyhydroborate (37.6 mg, 0.18 mmol) was added in one portion to a stirred suspension of 1-(heptadecan-9-yl) 13-nonyl 7-oxotridecanedioate (40.9 mg, 0.07 mmol), and 2-oxaspiro[3.3]heptan-6-aminium chloride (23.57 mg, 0.16 mmol) in 1,2-DCE (2 mL) and NMP (0.5 mL) under nitrogen. The resulting suspension was stirred at 25° C. for 40 hours. The reaction mixture was diluted with DCM (15 mL), water (5 mL) and sat. Na₂CO₃ (10 mL). The layers were separated, and the aqueous layer was extracted with DCM (3×15 mL). The combined organic layers were dried over MgSO₄, filtered and concentrated under reduced pressure to dryness to afford crude product. The resulting residue was purified by flash silica chromatography, elution gradient 0 to 50% of 20% MeOH/DCM (w/1% NH₄OH) in DCM. Product fractions were concentrated under reduced pressure to dryness to afford Compound 19 (0.023 g, 49.3%) as a colorless oil. ¹H NMR (500 MHz, Methanol-d4) δ ppm 0.9 (t, J=6.6 Hz, 9H) 1.3-1.4 (m, 48H) 1.5-1.6 (m, 4H) 1.6-1.7 (m, 6H) 1.9-2.0 (m, 2H) 2.3 (br t, J=7.2 Hz, 4H) 2.4-2.5 (m, 1H) 2.5-2.6 (m, 2H) 3.2 (t, J=7.9 Hz, 1H) 4.1 (t, J=6.6 Hz, 2H) 4.6 (s, 2H) 4.7 (s, 2H) 4.9-4.9 (m, 1H); C₄₅H₈₅NO₅ m/z calcd. 719.643 observed 720.8 [M+H]⁺ (LCMS).

### Example 20. Synthesis of Compound 20: 1-(heptadecan-9-yl) 13-nonyl 7-((oxetan-3-ylmethyl)amino)tridecanedioate

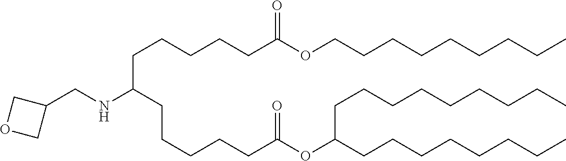 [SMILES: CCCCCCCCCOC(=O)CCCCCC(CCCCCC(=O)OC(CCCCCCCC)CCCCCCCCC)NCC1COC1]

Compound 20 was prepared according to the protocol described for Compound 19 using 13-(heptadecan-9-yloxy)-7,13-dioxotridecanoic acid and 1-(heptadecan-9-yl) 13-nonyl 7-oxotridecanedioate with the following additional steps. Sodium triacetoxyhydroborate (40.6 mg, 0.19 mmol) was added in one portion to a stirred suspension of 1-(heptadecan-9-yl) 13-nonyl 7-oxotridecanedioate (44.2 mg, 0.07 mmol), and oxetan-3-ylmethanaminium chloride (21.04 mg, 0.17 mmol) in 1,2-DCE (2 mL) and NMP (0.5 mL) under nitrogen. The resulting suspension was stirred at 25° C. for 40 hours. The reaction mixture was diluted with DCM (15 mL), water (5 mL) and sat. Na₂CO₃ (10 mL). The layers were separated, and the aqueous layer was extracted with DCM (3×15 mL). The combined organic layers were dried over MgSO₄, filtered and concentrated under reduced pressure to dryness to afford crude product. The resulting residue was purified by flash silica chromatography, elution gradient 0 to 35% of 20% MeOH/DCM (w/1% NH₄OH) in DCM. Product fractions were concentrated under reduced pressure to dryness to afford Compound 20 (0.023 g, 46.3%) as a colorless oil. ¹H NMR (500 MHz, Methanol-d4) δ ppm 0.9 (s, 9H) 1.3-1.4 (m, 44H) 1.4-1.5 (m, 4H) 1.5-1.6 (m, 4H) 1.6-1.7 (m, 6H) 2.3 (br t, J=7.2 Hz, 4H) 2.5 (br t, J=5.6 Hz, 1H) 2.9 (d, J=7.5 Hz, 2H) 3.1-3.1 (m, 1H) 4.1 (t, J=6.6 Hz, 2H) 4.4 (t, J=6.0 Hz, 2H) 4.8-4.8 (m, 2H) 4.9-4.9 (m, 1H); C₄₃H₈₃NO₅ m/z calcd. 693.627 observed 694.8 [M+H]+(LCMS).

Scheme 7 below illustrates the synthetic procedures for preparing Examples 21 and 22. In step t below, AcOH as an additive was used in reductive amination when utilizing the respective amines as a free base.

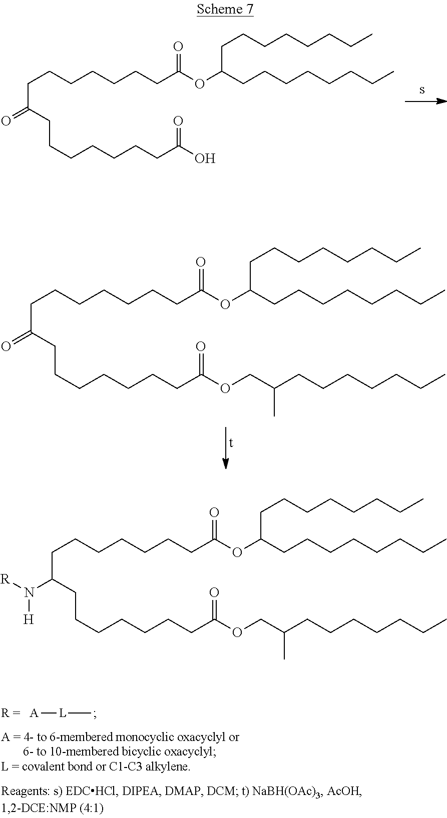

### Example 21. Synthesis of Compound 21: 1-(heptadecan-9-yl) 17-(2-methylnonyl) 9-(((tetrahydrofuran-3-yl)methyl)amino)heptadecanedioate

Step s): 3-(((ethylimino)methylene)amino)-N,N-dimethylpropan-1-amine hydrochloride (66.8 mg, 0.35 mmol) was added in one portion to a stirred solution of 2-methylnonan-1-ol (52.5 mg, 0.33 mmol), N-ethyl-N-isopropylpropan-2-amine (0.104 mL, 0.60 mmol), N,N-dimethylpyridin-4-amine (3.04 mg, 0.02 mmol) and 17-(heptadecan-9-yloxy)-9,17-dioxoheptadecanoic acid (91.7 mg, 0.17 mmol) in DCM (4 mL) at 0° C. under argon. The resulting solution was stirred at 25° C. for 16 hours. The reaction mixture was diluted with DCM (20 mL), 5% Citric acid solution (20 mL). The layers were separated, and the aqueous layer was extracted with DCM (3×20 mL). The combined organic layers were washed with saturated aqueous NaCl (20 mL). The organic layer was dried over MgSO₄, filtered and concentrated under reduced pressure to dryness to afford crude product. The resulting residue was purified by flash silica chromatography, elution gradient 0 to 35% EtOAc in hexanes. Product fractions were concentrated under reduced pressure to dryness to afford 1-(heptadecan-9-yl) 17-(2-methylnonyl) 9-oxoheptadecanedioate (87 mg, 76%) as a colorless oil. ¹H NMR (500 MHz, Chloroform-d) δ ppm 0.8-0.9 (m, 12H) 1.2-1.4 (m, 48H) 1.4-1.7 (m, 13H) 2.3 (dt, J=12.4, 7.5 Hz, 4H) 2.4 (t, J=7.5 Hz, 4H) 3.8-4.0 (m, 2H) 4.8-4.9 (m, 1H).

Step t):

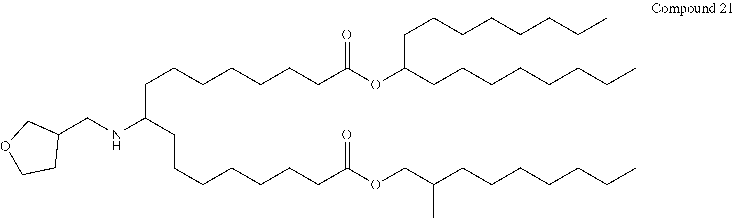 [SMILES: CCCCCCCCC(CCCCCCCC)OC(=O)CCCCCCCC(CCCCCCCC(=O)OCC(C)CCCCCCC)NCC1CCOC1]

sodium triacetoxyhydroborate (29.8 mg, 0.14 mmol) was added in one portion (after 10 min) to a stirred solution of 1-(heptadecan-9-yl) 17-(2-methylnonyl) 9-oxoheptadecanedioate (39 mg, 0.06 mmol), and (tetrahydrofuran-3-yl)methanamine (0.014 mL, 0.14 mmol) in 1,2-DCE (2 mL) and NMP (0.5 mL) under argon. The resulting solution was stirred at 25° C. for 18 hours. The reaction mixture was diluted with DCM (15 mL), water (5 mL) and Sat. Na₂CO₃ (10 mL). The layers were separated, and the aqueous layer was extracted with DCM (3×15 mL). The combined organic layers were dried over MgSO₄, filtered and evaporated to dryness to afford crude product. The resulting residue was purified by flash silica chromatography, elution gradient 0 to 40% of 20% MeOH in DCM (w/1% NH₄OH) in DCM. Product fractions were concentrated under reduced pressure to dryness to afford Compound 21 (30.3 mg, 69.2%) as a colorless oil. ¹H NMR (500 MHz, Methanol-d4) δ ppm 0.9-1.0 (m, 12H) 1.1-1.2 (m, 1H) 1.2-1.4 (m, 52H) 1.4-1.5 (m, 4H) 1.5-1.6 (m, 4H) 1.6-1.7 (m, 5H) 1.7-1.8 (m, 1H) 2.1-2.2 (m, 1H) 2.3-2.4 (m, 4H) 2.4-2.5 (m, 1H) 2.6-2.7 (m, 3H) 3.5 (dd, J=8.5, 6.2 Hz, 1H) 3.7-3.8 (m, 1H) 3.8-4.0 (m, 4H) 4.9-4.9 (m, 1H); C₄₉H₉₅NO₅ m/z calcd. 777.721 observed 778.8 [M+H]⁺ (LCMS).

### Example 22. Synthesis of Compound 22: 1-(heptadecan-9-yl) 17-(2-methylnonyl) 9-((2-oxaspiro[3.3]heptan-6-yl)amino)heptadecanedioate

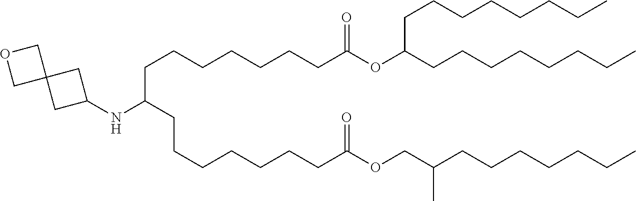 [SMILES: CCCCCCCCC(CCCCCCCC)OC(=O)CCCCCCCC(CCCCCCCC(=O)OCC(C)CCCCCCC)NC1CC2(COC2)C1]

Compound 22 was prepared according to the protocol described for Compound 21 using 1-(heptadecan-9-yl) 17-(2-methylnonyl) 9-oxoheptadecanedioate with the following additional steps. Sodium triacetoxyhydroborate (30.7 mg, 0.14 mmol) was added in one portion (after 10 min) to a stirred solution of 1-(heptadecan-9-yl) 17-(2-methylnonyl) 9-oxoheptadecanedioate (40.2 mg, 0.06 mmol), and 2-oxaspiro[3.3]heptan-6-aminium chloride (20.83 mg, 0.14 mmol) in 1,2-DCE (2 mL) and NMP (0.5 mL) under argon. The resulting solution was stirred at 25° C. for 18 hours. The reaction mixture was diluted with DCM (15 mL), water (5 mL) and Sat. Na₂CO₃ (10 mL). The layers were separated, and the aqueous layer was extracted with DCM (3×15 mL). The combined organic layers were dried over MgSO₄, filtered and evaporated to dryness to afford crude product. The resulting residue was purified by flash silica chromatography, elution gradient 0 to 40% of 20% MeOH in DCM (w/1% NH₄OH) in DCM. Product fractions were concentrated under reduced pressure to dryness to afford Compound 22 (12.10 mg, 26.4%) as a colorless oil. ¹H NMR (500 MHz, Methanol-d4) δ ppm 0.9-1.0 (m, 12H) 1.3-1.4 (m, 56H) 1.5-1.6 (m, 4H) 1.6-1.7 (m, 4H) 1.7-1.8 (m, 1H) 2.0-2.0 (m, 2H) 2.3-2.3 (m, 4H) 2.5-2.6 (m, 3H) 3.2-3.2 (m, 1H) 3.9-4.0 (m, 2H) 4.6-4.6 (m, 2H) 4.7-4.7 (m, 2H) 4.9-4.9 (m, 1H); C₅₀H₉₅NO₅ m/z calcd. 789.721 observed 790.8 [M+H]+ (LCMS).

Scheme 8 below illustrates the synthetic procedures for preparing Examples 23 and 24.

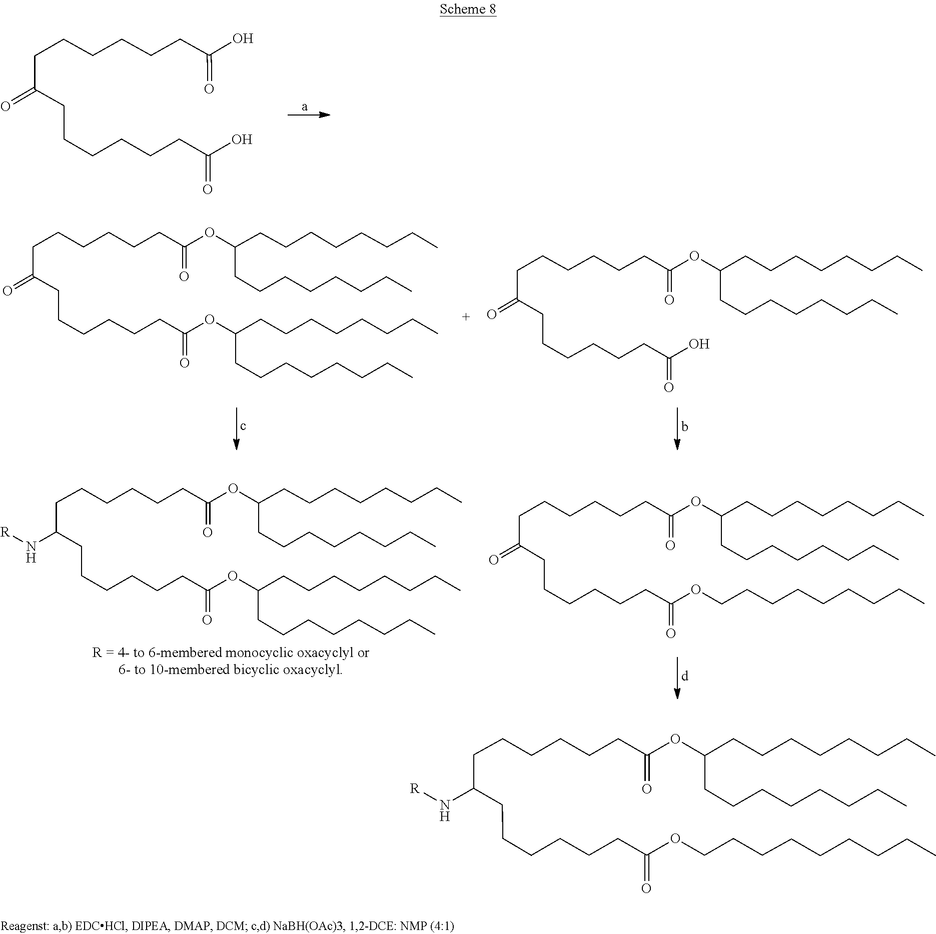

### Example 23. Synthesis of Compound 23: di(heptadecan-9-yl) 8-((2-oxaspiro[3.3]heptan-6-yl)amino)pentadecanedioate

### Step 1: di(heptadecan-9-yl) 8-oxopentadecanedioate and 15-(heptadecan-9-yloxy)-8,15-dioxopentadecanoic acid

EDC (0.201 g, 1.05 mmol) was added in one portion to a stirred solution of 9-heptadecanol (0.143 g, 0.56 mmol), 8-oxopentadecanedioic acid (0.200 g, 0.70 mmol), DIPEA (0.378 mL, 2.17 mmol), and DMAP (0.017 g, 0.14 mmol) in DCM (5 mL) at 0° C. under argon. The resulting solution was stirred at room temperature for 16 hours. The reaction mixture was concentrated under reduced pressure to dryness and redissolved in EtOAc (20 mL) and sodium biocarbonate (20 mL). The layers were separated, and the aqueous layer was extracted with (EtOAc) (3×30 mL). The combined organic layers were washed sequentially with 5% citric acid (25 mL) and saturated aqueous NaCl (25 mL). The organic layer was dried over MgSO₄, filtered and concentrated under reduced pressure to dryness to afford crude product. The resulting residue was purified by flash silica chromatography, elution gradient 0 to 60% EtOAc in hexanes. Product fractions were concentrated under reduced pressure to dryness to afford di(heptadecan-9-yl) 8-oxopentadecanedioate (0.064 g, 11.99%) and 15-(heptadecan-9-yloxy)-8,15-dioxopentadecanoic acid (0.102 g, 27.8%) as a colorless oils. Di(heptadecan-9-yl) 8-oxopentadecanedioate: ¹H NMR (500 MHz, CHLOROFORM-d, 22° C.) δ ppm 0.88 (12H, t), 1.26 (54H, m), 1.57 (18H, m), 2.24-2.31 (4H, t), 2.34-2.44 (4H, t), 4.87 (2H, m). 15-(heptadecan-9-yloxy)-8,15-dioxopentadecanoic acid: ¹H NMR (500 MHz, CHLOROFORM-d, 22° C.) δ ppm 0.84-0.94 (6H, t), 1.20-1.70 (44H, m), 2.24-2.45 (8H, m), 4.87 (1H, m).

### Step 2: 1-(heptadecan-9-yl) 15-nonyl 8-oxopentadecanedioate

EDC (0.054 g, 0.28 mmol) was added in one portion to a stirred solution of 15-(heptadecan-9-yloxy)-8,15-dioxopentadecanoic acid (0.071 g, 0.14 mmol)), nonan-1-ol (0.035 mL, 0.20 mmol), DIPEA (0.073 mL, 0.42 mmol), and DMAP (3.31 mg, 0.03 mmol) in DCM (5 mL) at 0° C. under argon. The resulting solution was stirred at room temperature for 16 hours. The reaction mixture was concentrated under reduced pressure to dryness and redissolved in EtOAc (20 mL) and sodium biocarbonate (20 mL). The layers were separated, and the aqueous layer was extracted with (EtOAc) (3×30 mL). The organic layer was dried over MgSO₄, filtered and concentrated under reduced pressure to dryness to afford crude product. The resulting residue was purified by flash silica chromatography, elution gradient 0 to 60% EtOAc in hexanes. Product fractions were concentrated under reduced pressure to dryness to afford 1-(heptadecan-9-yl) 15-nonyl 8-oxopentadecanedioate (0.063 g, 71.5%) as a colorless oil. ¹H NMR (500 MHz, CHLOROFORM-d, 27° C.) δ ppm 0.83-0.94 (9H, m), 1.21-1.68 (58H, m), 2.22-2.33 (4H, m), 2.38 (4H, t), 4.06 (2H, t), 4.87 (1H, m).

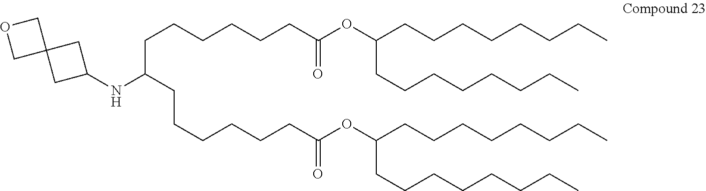 [SMILES: CCCCCCCCC(CCCCCCCC)OC(=O)CCCCCCC(CCCCCCC(=O)OC(CCCCCCCC)CCCCCCCC)NC1CC2(COC2)C1]

Sodium triacetoxyborohydride (0.055 g, 0.26 mmol) was added in one portion to a stirred solution of 2-oxaspiro[3.3]heptan-6-amine hydrochloride (0.038 g, 0.25 mmol) and di(heptadecan-9-yl) 8-oxopentadecanedioate (0.0639 g, 0.08 mmol) in DCE (4 mL) and NMP (1 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford di(heptadecan-9-yl) 8-((2-oxaspiro[3.3]heptan-6-yl)amino)pentadecanedioate (0.046 g, 64.3%) as a colorless oil. ¹H NMR (500 MHz, CHLOROFORM-d, 22° C.) δ ppm 0.83-0.94 (12H, m), 1.19-1.39 (62H, m), 1.51 (10H, m), 1.58-1.66 (4H, m), 1.83 (2H, m), 2.28 (4H, t), 2.37-2.44 (1H, m), 2.51-2.58 (2H, m), 3.03-3.20 (1H, m), 4.57-4.62 (2H, s), 4.71 (2H, s), 4.87 (2H, m); C44H87NO5 m/z calcd. 859.799 observed 861.0 [M+H]+(LCMS).

### Example 24. Synthesis of Compound 24: 1-(heptadecan-9-yl) 15-nonyl 8-((2-oxaspiro[3.3]heptan-6-yl)amino)pentadecanedioate

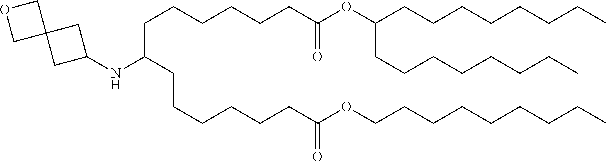 [SMILES: CCCCCCCCCOC(=O)CCCCCCC(CCCCCCC(=O)OC(CCCCCCCC)CCCCCCCC)NC1CC2(COC2)C1]

Compound 24 was prepared according to the protocol described for Compound 23 using 15-(heptadecan-9-yloxy)-8,15-dioxopentadecanoic acid and 1-(heptadecan-9-yl) 15-nonyl 8-oxopentadecanedioate with the following additional steps. Sodium triacetoxyborohydride (0.062 g, 0.29 mmol) was added in one portion to a stirred solution of 2-oxaspiro[3.3]heptan-6-amine hydrochloride (0.043 g, 0.29 mmol) and 1-(heptadecan-9-yl) 15-nonyl 8-oxopentadecanedioate (0.063 g, 0.10 mmol) in DCE (4 mL) and NMP (1 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford Compound 24 (0.036 g, 49.7%) as a colorless oil. ¹H NMR (500 MHz, CHLOROFORM-d, 22° C.) δ ppm 0.82-0.96 (9H, m), 1.28 (62H, m), 1.81-1.91 (2H, m), 2.25-2.35 (4H, m), 2.39-2.48 (1H, m), 2.52-2.60 (2H, m), 3.11-3.20 (1H, m), 4.08 (2H, t), 4.62 (2H, s), 4.73 (2H, s), 4.89 (1H, m); C47H89NO5 m/z calcd. 747.674 observed 748.8 [M+H]+ (LCMS).

Scheme 9 below illustrates the synthetic procedures for preparing Example 25.

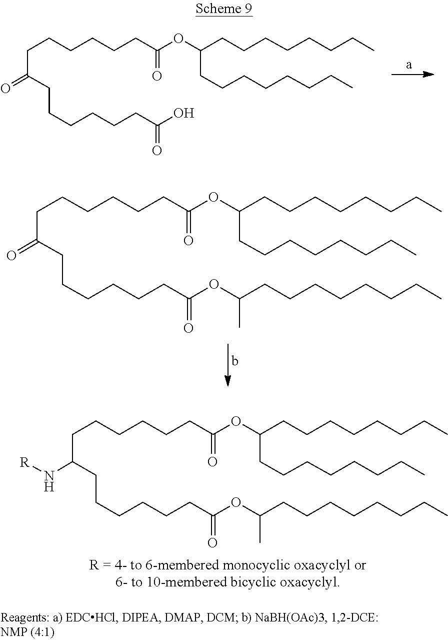

### Example 25. Synthesis of Compound 25: 1-(decan-2-yl) 15-(heptadecan-9-yl) 8-((2-oxaspiro[3.3]heptan-6-yl)amino)pentadecanedioate

### Step 1: 1-(decan-2-yl) 15-(heptadecan-9-yl) 8-oxopentadecanedioate

EDC (141 mg, 0.73 mmol) was added in one portion to a stirred mixture of 15-(heptadecan-9-yloxy)-8,15-dioxopentadecanoic acid (124.1 mg, 0.24 mmol), decan-2-ol (0.055 mL, 0.50 mmol), DIPEA (0.169 mL, 0.97 mmol), and DMAP (5.78 mg, 0.05 mmol) in DCM (5 mL) at 0° C. under argon. The resulting mixture was stirred at room temperature for 16 hours. The reaction mixture was diluted with sodium bicarbonate (25 mL) and DCM (25 mL). The layers were separated, and the aqueous layer was extracted with (DCM) (4×25 mL). The combined organic layers were dried over MgSO₄, filtered and concentrated under reduced pressure to dryness to afford crude product. The resulting residue was purified by flash silica chromatography, elution gradient 0 to 40% EtOAc in hexanes. Product fractions were concentrated under reduced pressure to dryness to afford 1-(decan-2-yl) 15-(heptadecan-9-yl) 8-oxopentadecanedioate (87 mg, 55.3%) as a colorless oil. ¹H NMR (500 MHz, CHLOROFORM-d, 22° C.) δ ppm 0.89 (9H, t), 1.16-1.70 (61H, m), 2.27 (4H, m), 2.33-2.45 (4H, m), 4.80-4.97 (2H, m).

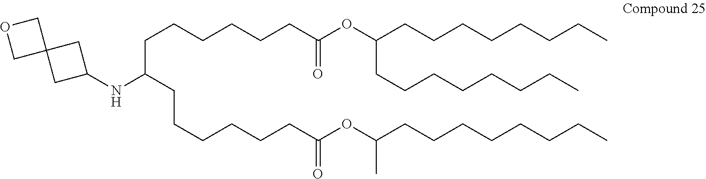 [SMILES: CCCCCCCCC(C)OC(=O)CCCCCCC(CCCCCCC(=O)OC(CCCCCCCC)CCCCCCCC)NC1CC2(COC2)C1]

Sodium triacetoxyborohydride (25.8 mg, 0.12 mmol) was added in one portion to a stirred solution of 2-oxaspiro[3.3]heptan-6-amine hydrochloride (16.20 mg, 0.11 mmol) and 1-(decan-2-yl) 15-(heptadecan-9-yl) 8-oxopentadecanedioate (30 mg, 0.05 mmol) in DCE (2 mL) and NMP (0.5 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford 1-(decan-2-yl) 15-(heptadecan-9-yl) 8-((2-oxaspiro[3.3]heptan-6-yl)amino)pentadecanedioate (18.20 mg, 52.9%) as a colorless oil. ¹H NMR (500 MHz, METHANOL-d4, 27° C.) δ ppm 0.92 (9H, t), 1.19-1.71 (65H, m), 1.93-2.08 (2H, m), 2.26-2.38 (4H, m), 2.50-2.61 (3H, m), 3.17-3.29 (1H, m), 4.61 (2H, s), 4.74 (2H, s), 4.87-4.94 (2H, m); C48H91 NO5 m/z calcd. 761.690 observed 762.7 [M+H]+ (LCMS).

Scheme 10 below illustrates the synthetic procedures for preparing Example 26.

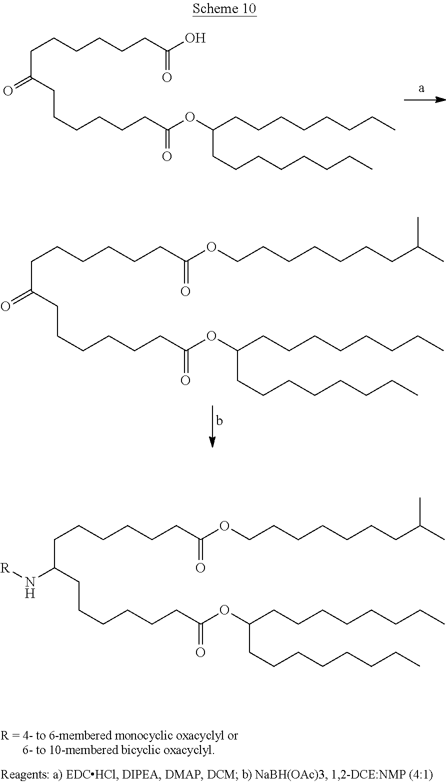

### Example 26. Synthesis of Compound 26: 1-(heptadecan-9-yl) 15-(8-methylnonyl) 8-((2-oxaspiro[3.3]heptan-6-yl)amino)pentadecanedioate

### Step 1: 1-(heptadecan-9-yl) 15-(8-methylnonyl) 8-oxopentadecanedioate

EDC (0.023 g, 0.12 mmol) was added in one portion to a stirred solution of 15-(heptadecan-9-yloxy)-8,15-dioxopentadecanoic acid (0.03 g, 0.06 mmol)), 8-methylnonan-1-ol (0.019 g, 0.12 mmol), DIPEA (0.031 mL, 0.18 mmol), and DMAP (1.397 mg, 0.01 mmol) in DCM (5 mL) at 0° C. under argon. The resulting solution was stirred at room temperature for 16 hours. The reaction mixture was concentrated under reduced pressure to dryness and redissolved in EtOAc (20 mL) and sodium bicarbonate (20 mL). The layers were separated, and the aqueous layer was extracted with (EtOAc) (3×30 mL). The combined organic layers were washed sequentially with 5% citric acid (25 mL) and saturated aqueous NaCl (25 mL). The organic layer was dried over MgSO₄, filtered and concentrated under reduced pressure to dryness to afford crude product. The resulting residue was purified by flash silica chromatography, elution gradient 0 to 60% EtOAc in hexanes. Product fractions were concentrated under reduced pressure to dryness to afford 1-(heptadecan-9-yl) 15-(8-methylnonyl) 8-oxopentadecanedioate (0.028 g, 72.3%) as a colorless oils. ¹H NMR (500 MHz, CHLOROFORM-d, 23° C.) δ ppm 0.82-0.93 (12H, m), 1.10-1.19 (2H, m), 1.21-1.40 (40H, m), 1.46-1.68 (15H, m), 2.25-2.34 (4H, m), 2.38 (4H, t), 4.06 (2H, t), 4.80-4.94 (1H, m).

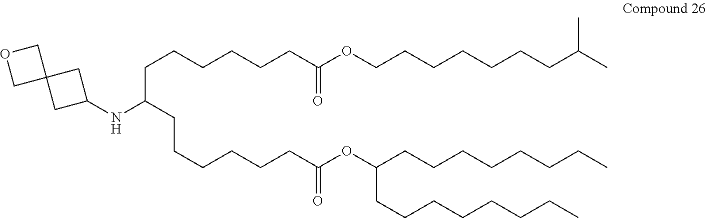 [SMILES: CCCCCCCCC(CCCCCCCC)OC(=O)CCCCCCC(CCCCCCC(=O)OCCCCCCCC(C)C)NC1CC2(COC2)C1]

Sodium triacetoxyborohydride (0.026 g, 0.12 mmol) was added in one portion to a stirred solution of 2-oxaspiro[3.3]heptan-6-amine hydrochloride (0.019 g, 0.12 mmol) and 1-(heptadecan-9-yl) 15-(8-methylnonyl) 8-oxopentadecanedioate (0.0275 g, 0.04 mmol) in DCE (4 mL) and NMP (1 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford 1-(heptadecan-9-yl) 15-(8-methylnonyl) 8-((2-oxaspiro[3.3]heptan-6-yl)amino)pentadecanedioate (0.023 g, 72.3%) as a colorless oil. ¹H NMR (500 MHz, CHLOROFORM-d, 27° C.) δ ppm 0.84-0.93 (12H, m), 1.12-1.68 (61H, m), 1.79-1.91 (2H, m), 2.29 (4H, m), 2.38-2.47 (1H, m), 2.50-2.59 (2H, m), 3.07-3.18 (1H, m), 4.06 (2H, t), 4.60 (2H, s), 4.71 (2H, s), 4.87 (1H, m); C48H91 NO5 m/z calcd. 761.690 observed 762.9 [M+H]+ (LCMS).

Scheme 11 below illustrates the synthetic procedures for preparing Example 27.

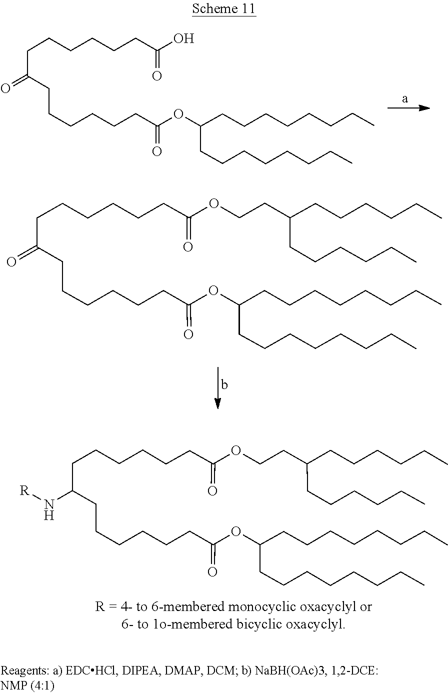

### Example 27. Synthesis of Compound 27: 1-(heptadecan-9-yl) 15-(3-heptyldecyl) 8-((2-oxaspiro[3.3]heptan-6-yl)amino)pentadecanedioate

### Step 1: 1-(heptadecan-9-yl) 15-(3-hexylnonyl) 8-oxopentadecanedioate

EDC (46.0 mg, 0.24 mmol) was added in one portion to a stirred mixture of 15-(heptadecan-9-yloxy)-8,15-dioxopentadecanoic acid (60 mg, 0.11 mmol), 3-heptyldecan-1-ol (44.0 mg, 0.17 mmol), DIPEA (0.082 mL, 0.47 mmol), and DMAP (2.79 mg, 0.02 mmol) in DCM (5 mL) at 0° C. under argon. The resulting mixture was stirred at room temperature for 16 hours. The reaction mixture was diluted with sodium bicarbonate (25 mL) and DCM (25 mL). The layers were separated, and the aqueous layer was extracted with (DCM) (4×25 mL). The combined organic layers were dried over MgSO₄, filtered and concentrated under reduced pressure to dryness to afford crude product. The resulting residue was purified by flash silica chromatography, elution gradient 0 to 20% EtOAc in hexanes. Product fractions were concentrated under reduced pressure to dryness to afford 1-(heptadecan-9-yl) 15-(3-heptyldecyl) 8-oxopentadecanedioate (60.0 mg, 68.8%) as a colorless oil. ¹H NMR (500 MHz, CHLOROFORM-d, 21° C.) δ ppm 0.89 (12H, m), 1.26 (71H, m), 2.24-2.32 (4H, m), 2.34-2.42 (4H, t), 4.03-4.13 (2H, t), 4.81-4.91 (1H, m).

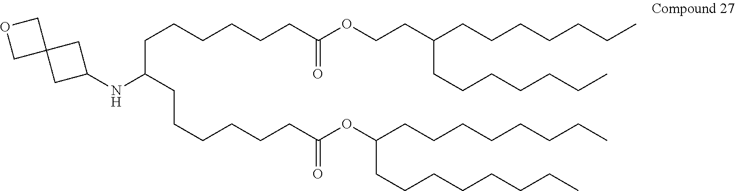 [SMILES: CCCCCCCCC(CCCCCCCC)OC(=O)CCCCCCC(CCCCCCC(=O)OCCC(CCCCCCC)CCCCCCC)NC1CC2(COC2)C1]

Sodium triacetoxyborohydride (0.019 g, 0.09 mmol) was added in one portion to a stirred solution of 2-oxaspiro[3.3]heptan-6-amine hydrochloride (0.013 g, 0.09 mmol) and 1-(heptadecan-9-yl) 15-(3-heptyldecyl) 8-oxopentadecanedioate (0.0223 g, 0.03 mmol) in DCE (4 mL) and NMP (1 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford 1-(heptadecan-9-yl) 15-(3-heptyldecyl) 8-((2-oxaspiro[3.3]heptan-6-yl)amino)pentadecanedioate (0.014 g, 55.7%) as a colorless oil. ¹H NMR (500 MHz, CHLOROFORM-d, 27° C.) δ ppm 0.89 (12H, m), 1.20-1.68 (75H, m), 1.81-1.93 (2H, m), 2.23-2.34 (4H, m), 2.40-2.49 (1H, m), 2.51-2.60 (2H, m), 3.07-3.21 (1H, m), 4.05-4.12 (2H, t), 4.60 (2H, s), 4.71 (2H, s), 4.82-4.92 (1H, m); C55H105NO5 m/z calcd. 859.799 observed 861.0 [M+H]+ (LCMS).

Scheme 12 below illustrates the synthetic procedures for preparing Examples 28 and 29.

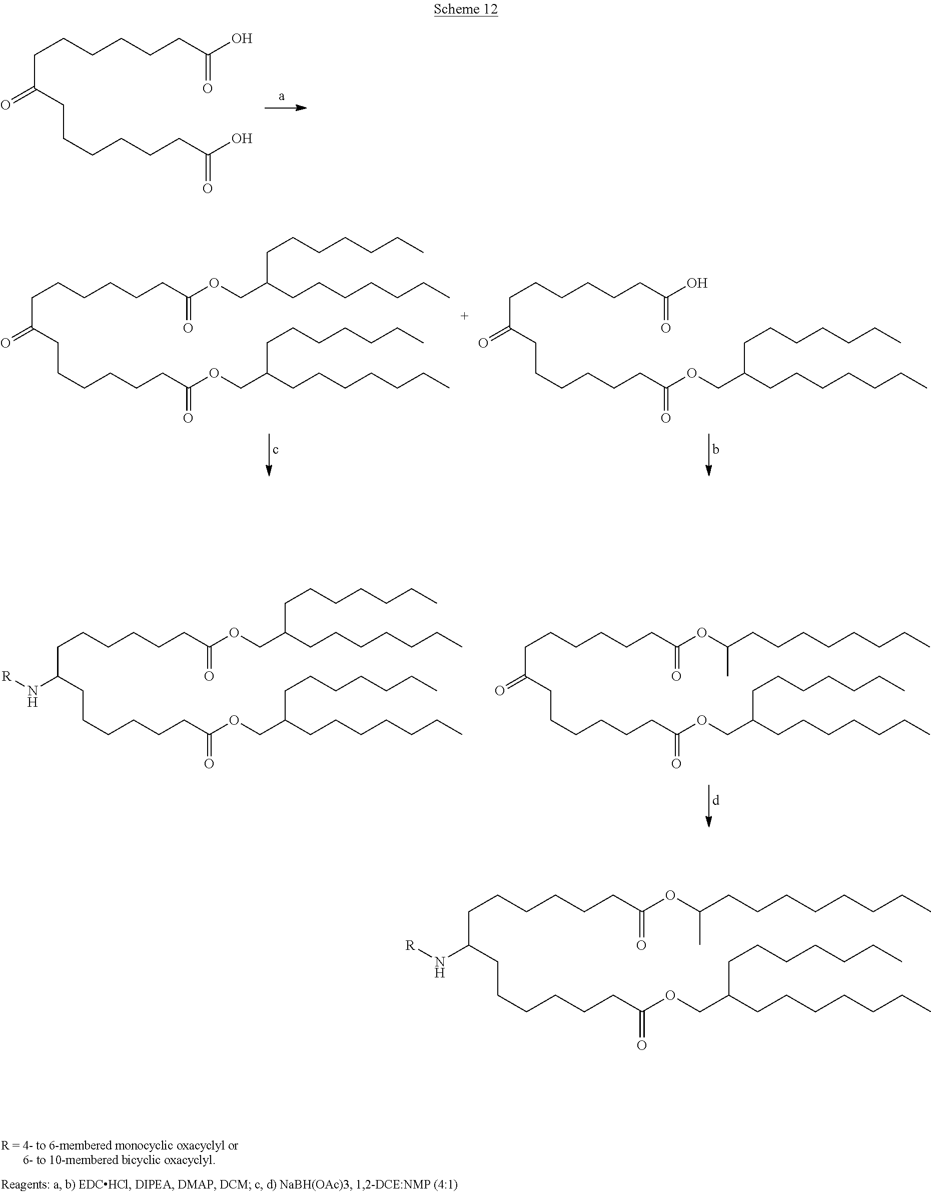

### Example 28. Synthesis of Compound 28: Bis(2-heptylnonyl) 8-((2-oxaspiro[3.3]heptan-6-yl)amino)pentadecanedioate

### Step 1: bis(2-heptylnonyl) 8-oxopentadecanedioate and 15-((2-heptylnonyl)oxy)-8,15-dioxopentadecanoic acid

EDC (0.201 g, 1.05 mmol) was added in one portion to a stirred solution of 2-heptylnonan-1-ol (0.135 g, 0.56 mmol)),8-oxopentadecanedioic acid (0.200 g, 0.70 mmol), DIPEA (0.378 mL, 2.17 mmol), and DMAP (0.017 g, 0.14 mmol) in DCM (5 mL) at 0° C. under argon. The resulting solution was stirred at room temperature for 16 hours. The reaction mixture was concentrated under reduced pressure to dryness and redissolved in EtOAc (20 mL) and sodium bicarbonate (20 mL). The layers were separated, and the aqueous layer was extracted with (EtOAc) (3×30 mL). The combined organic layers were washed sequentially with 5% citric acid (25 mL) and saturated aqueous NaCl (25 mL). The organic layer was dried over MgSO₄, filtered and concentrated under reduced pressure to dryness to afford crude product. The resulting residue was purified by flash silica chromatography, elution gradient 0 to 60% EtOAc in hexanes. Product fractions were concentrated under reduced pressure to dryness to afford bis(2-heptylnonyl) 8-oxopentadecanedioate (0.060 g, 11.72%) and 15-((2-heptylnonyl)oxy)-8,15-dioxopentadecanoic acid (0.126 g, 35.4%) as a colorless oils. Bis(2-heptylnonyl) 8-oxopentadecanedioate: ¹H NMR (500 MHz, CHLOROFORM-d, 22° C.) δ ppm 0.83-0.95 (12H, m), 1.19-1.37 (56H, m), 1.62 (10H, m), 2.25-2.34 (4H, t), 2.38 (4H, t), 3.97 (4H, d). 15-((2-heptylnonyl)oxy)-8,15-dioxopentadecanoic acid: ¹H NMR (500 MHz, CHLOROFORM-d, 22° C.) δ ppm 0.83-0.95 (6H, m), 1.27 (32H, br m), 1.52-1.72 (10H, m), 2.24-2.45 (8H, m), 3.97 (2H, d).

### Step 2: 1-(decan-2-yl) 15-(2-heptylnonyl) 8-oxopentadecanedioate

EDC (0.055 g, 0.29 mmol) was added in one portion to a stirred solution of 15-((2-heptylnonyl)oxy)-8,15-dioxopentadecanoic acid (0.0703 g, 0.14 mmol)), decan-2-ol (0.040 mL, 0.21 mmol), N-ethyl-N-isopropylpropan-2-amine (0.072 mL, 0.41 mmol), and N,N-dimethylpyridin-4-amine (3.36 mg, 0.03 mmol) in DCM (10 mL) at 0° C. under argon. The resulting solution was stirred at room temperature for 16 hours. The reaction mixture was concentrated under reduced pressure to dryness and redissolved in EtOAc (20 mL) and sodium bicarbonate (20 mL). The layers were separated, and the aqueous layer was extracted with (EtOAc) (3×30 mL). The organic layer was dried over MgSO₄, filtered and concentrated under reduced pressure to dryness to afford crude product. The resulting residue was purified by flash silica chromatography, elution gradient 0 to 60% EtOAc in hexanes. Product fractions were concentrated under reduced pressure to dryness to afford 1-(decan-2-yl) 15-(2-heptylnonyl) 8-oxopentadecanedioate (0.041 g, 46.2%) as a colorless oil. ¹H NMR (500 MHz, CHLOROFORM-d, 21° C.) δ ppm 0.84-0.93 (9H, m), 1.15-1.70 (58H, m), 2.23-2.32 (4H, m), 2.38 (4H, t), 3.94-3.99 (2H, d), 4.81-4.95 (1H, m).

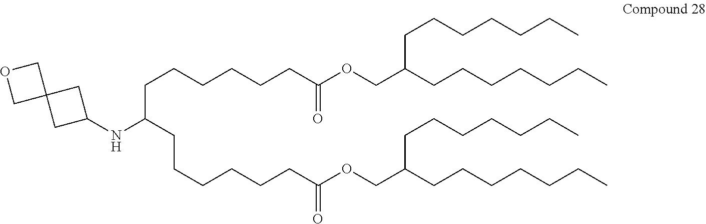 [SMILES: CCCCCCCC(CCCCCCC)COC(=O)CCCCCCC(CCCCCCC(=O)OCC(CCCCCCC)CCCCCCC)NC1CC2(COC2)C1]

Sodium triacetoxyborohydride (0.020 g, 0.10 mmol) was added in one portion to a stirred solution of 2-oxaspiro[3.3]heptan-6-amine hydrochloride (0.013 g, 0.08 mmol) and bis(2-heptylnonyl) 8-oxopentadecanedioate (0.026 g, 0.04 mmol) in DCE (2 mL) and NMP (0.5 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford bis(2-heptylnonyl) 8-((2-oxaspiro[3.3]heptan-6-yl)amino)pentadecanedioate (0.018 g, 60.8%) as a colorless oil. ¹H NMR (500 MHz, CHLOROFORM-d, 21° C.) δ ppm 0.84-0.93 (12H, m), 1.20-1.41 (64H, m), 1.56-1.68 (6H, m), 1.84-1.99 (2H, m), 2.30 (4H, sm, 2.41-2.49 (1H, m), 2.52-2.59 (2H, m), 3.12-3.21 (1H, m), 3.97 (4H, d), 4.60 (2H, s), 4.71 (2H, s); C53H101 NO5 m/z calcd. 831.768 observed 833.0 [M+H]+ (LCMS).

### Example 29. Synthesis of Compound 29: 1-(decan-2-yl) 15-(2-heptylnonyl) 8-((2-oxaspiro[3.3]heptan-6-yl)amino)pentadecanedioate

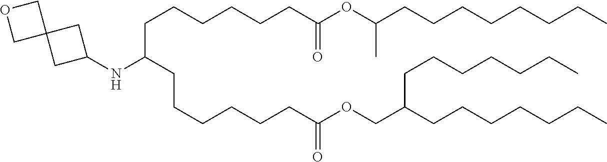 [SMILES: CCCCCCCCC(C)OC(=O)CCCCCCC(CCCCCCC(=O)OCC(CCCCCCC)CCCCCCC)NC1CC2(COC2)C1]

Compound 29 was prepared according to the protocol described for Compound 28 using 15-((2-heptylnonyl)oxy)-8,15-dioxopentadecanoic acid and 1-(decan-2-yl) 15-(2-heptylnonyl) 8-oxopentadecanedioate with the following additional steps. Sodium triacetoxyborohydride (0.036 g, 0.17 mmol) was added in one portion to a stirred solution of 2-oxaspiro[3.3]heptan-6-amine hydrochloride (0.023 g, 0.15 mmol) and 1-(decan-2-yl) 15-(2-heptylnonyl) 8-oxopentadecanedioate (0.0414 g, 0.06 mmol) in DCE (2 mL) and NMP (0.5 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford 1-(decan-2-yl) 15-(2-heptylnonyl) 8-((2-oxaspiro[3.3]heptan-6-yl)amino)pentadecanedioate (0.028 g, 59.5%) as a colorless oil. ¹H NMR (500 MHz, CHLOROFORM-d, 27° C.) δ ppm 0.86-0.93 (9H, m), 1.16-1.74 (62H, m), 1.82-1.98 (2H, m), 2.24-2.33 (4H, m), 2.39-2.48 (1H, m), 2.51-2.59 (2H, m), 3.10-3.22 (1H, m), 3.94-4.01 (2H, m), 4.60 (2H, s), 4.71 (2H, s), 4.85-4.94 (1H, m); C47H89NO5 m/z calcd. 747.674 observed 748.9 [M+H]+ (LCMS).

Scheme 13 below illustrates the synthetic procedures for preparing Example 30.

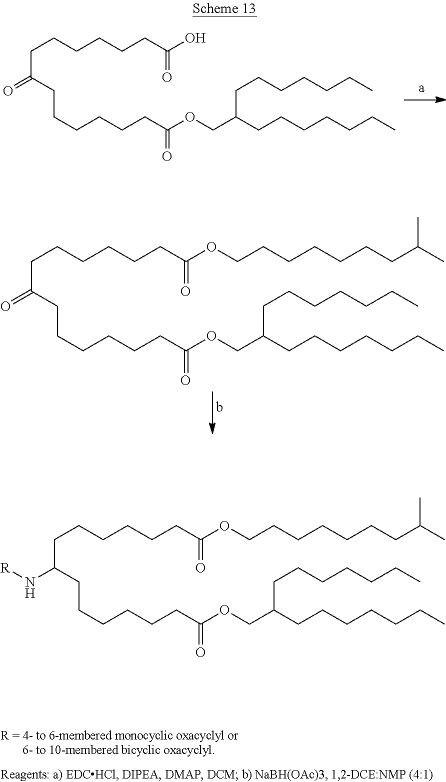

### Example 30. Synthesis of Compound 30: 1-(2-heptylnonyl) 15-(8-methylnonyl) 8-((2-oxaspiro[3.3]heptan-6-yl)amino)pentadecanedioate

### Step 1: 1-(2-heptylnonyl) 15-(8-methylnonyl) 8-oxopentadecanedioate

EDC (0.047 g, 0.25 mmol) was added in one portion to a stirred solution of 15-((2-heptylnonyl)oxy)-8,15-dioxopentadecanoic acid (0.060 g, 0.12 mmol)), 8-methylnonan-1-ol (0.039 g, 0.25 mmol), DIPEA (0.064 mL, 0.36 mmol), and DMAP (2.87 mg, 0.02 mmol) in DCM (5 mL) at 0° C. under argon. The resulting solution was stirred at room temperature for 16 hours. The reaction mixture was concentrated under reduced pressure to dryness and redissolved in EtOAc (20 mL) and sodium bicarbonate (20 mL). The layers were separated, and the aqueous layer was extracted with (EtOAc) (3×30 mL). The organic layer was dried over MgSO₄, filtered and concentrated under reduced pressure to dryness to afford crude product. The resulting residue was purified by flash silica chromatography, elution gradient 0 to 60% EtOAc in hexanes. Product fractions were concentrated under reduced pressure to dryness to afford 1-(2-heptylnonyl) 15-(8-methylnonyl) 8-oxopentadecanedioate (0.071 g, 93%) as a colorless oil. ¹H NMR (500 MHz, CHLOROFORM-d, 27° C.) δ ppm 0.87 (12H, m), 1.12-1.67 (54H, m), 2.28 (4H, m), 2.34-2.41 (4H, m), 3.96 (2H, d), 4.05 (2H, t).

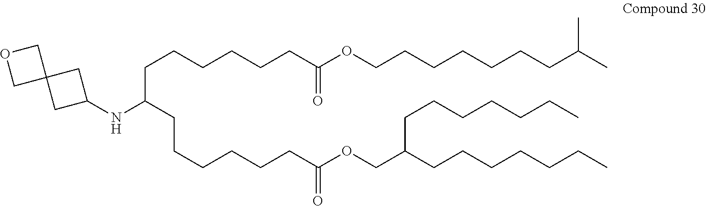 [SMILES: CCCCCCCC(CCCCCCC)COC(=O)CCCCCCC(CCCCCCC(=O)OCCCCCCCC(C)C)NC1CC2(COC2)C1]

Sodium triacetoxyborohydride (0.069 g, 0.33 mmol) was added in one portion to a stirred solution of 2-oxaspiro[3.3]heptan-6-amine hydrochloride (0.049 g, 0.33 mmol) and 1-(2-heptylnonyl) 15-(8-methylnonyl) 8-oxopentadecanedioate (0.071 g, 0.11 mmol) in DCE (4 mL) and NMP (1 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified twice by flash silica chromatography, elution gradient 0 to 100% (20% MeOH and 1% NH₄OH in DCM) in DCM. Product fractions were concentrated under reduced pressure to dryness to afford 1-(2-heptylnonyl) 15-(8-methylnonyl) 8-((2-oxaspiro[3.3]heptan-6-yl)amino)pentadecanedioate (0.033 g, 40.1%) as a colorless oil. ¹H NMR (500 MHz, CHLOROFORM-d, 27° C.) δ ppm 0.84-0.92 (12H, m), 1.12-1.69 (58H, m), 1.81-1.92 (2H, m), 2.24-2.34 (4H, m), 2.38-2.45 (1H, m), 2.50-2.59 (2H, m), 3.07-3.20 (1H, m), 3.95-3.99 (2H, m), 4.06 (2H, t), 4.60 (2H, s), 4.71 (2H, s); C₄₇H89NO5 m/z calcd. 747.674 observed 748.8 [M+H]+ (LCMS).

Scheme 14 below illustrates the synthetic procedures for preparing Examples 31 and 32.

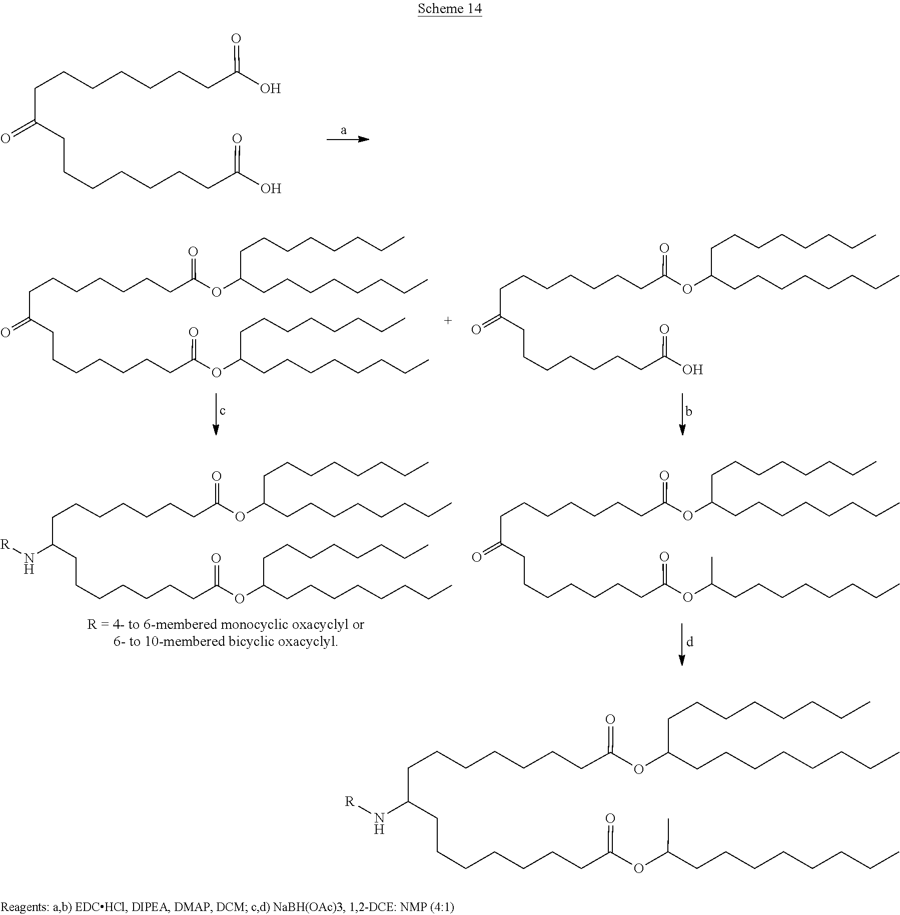

### Example 31. Synthesis of Compound 31: Di(heptadecan-9-yl) 9-((2-oxaspiro[3.3]heptan-6-yl)amino)heptadecanedioate

### Step 1: di(heptadecan-9-yl) 9-oxoheptadecanedioate and 17-(heptadecan-9-yloxy)-9,17-dioxoheptadecanoic acid

EDC (0.137 g, 0.72 mmol) was added in one portion to a stirred solution of 9-heptadecanol (0.098 g, 0.38 mmol)),9-oxoheptadecanedioic acid (0.15 g, 0.48 mmol), DIPEA (0.258 mL, 1.48 mmol), and DMAP (0.012 g, 0.10 mmol) in DCM (5 mL) at 0° C. under argon. The resulting solution was stirred at room temperature for 16 hours. The reaction mixture was concentrated under reduced pressure to dryness and redissolved in EtOAc (20 mL) and sodium biocarbonate (20 mL). The layers were separated, and the aqueous layer was extracted with (EtOAc) (3×30 mL). The combined organic layers were washed sequentially with 5% citric acid (25 mL) and saturated aqueous NaCl (25 mL). The organic layer was dried over MgSO₄, filtered and concentrated under reduced pressure to dryness to afford crude product. The resulting residue was purified by flash silica chromatography, elution gradient 0 to 60% EtOAc in hexanes. Product fractions were concentrated under reduced pressure to dryness to afford di(heptadecan-9-yl) 9-oxoheptadecanedioate (0.047 g, 12.50%) and 17-(heptadecan-9-yloxy)-9,17-dioxoheptadecanoic acid (0.105 g, 39.7%) as a colorless oil. di(heptadecan-9-yl) 9-oxoheptadecanedioate: ¹H NMR (500 MHz, CHLOROFORM-d, 22° C.) δ ppm 0.88 (12H, t), 1.18-1.68 (76H, m), 2.23-2.32 (4H, t), 2.35-2.42 (4H, t), 4.87 (2H, m). 17-(heptadecan-9-yloxy)-9,17-dioxoheptadecanoic acid: ¹H NMR (500 MHz, CHLOROFORM-d, 22° C.) δ ppm 0.83-0.95 (6H, m), 1.18-1.39 (36H, m), 1.45-1.68 (12H, m), 2.38 (8H, m), 4.77-4.96 (1H, m).

### Step 2: 1-(decan-2-yl) 15-(2-heptylnonyl) 8-oxopentadecanedioate

EDC (0.055 g, 0.29 mmol) was added in one portion to a stirred solution of 15-((2-heptylnonyl)oxy)-8,15-dioxopentadecanoic acid (0.0703 g, 0.14 mmol)), decan-2-ol (0.040 mL, 0.21 mmol), N-ethyl-N-isopropylpropan-2-amine (0.072 mL, 0.41 mmol), and N,N-dimethylpyridin-4-amine (3.36 mg, 0.03 mmol) in DCM (10 mL) at 0° C. under argon. The resulting solution was stirred at room temperature for 16 hours. The reaction mixture was concentrated under reduced pressure to dryness and redissolved in EtOAc (20 mL) and sodium bicarbonate (20 mL). The layers were separated, and the aqueous layer was extracted with (EtOAc) (3×30 mL). The organic layer was dried over MgSO₄, filtered and concentrated under reduced pressure to dryness to afford crude product. The resulting residue was purified by flash silica chromatography, elution gradient 0 to 60% EtOAc in hexanes. Product fractions were concentrated under reduced pressure to dryness to afford 1-(decan-2-yl) 15-(2-heptylnonyl) 8-oxopentadecanedioate (0.041 g, 46.2%) as a colorless oil. ¹H NMR (500 MHz, CHLOROFORM-d, 21° C.) δ ppm 0.84-0.93 (9H, m), 1.15-1.70 (58H, m), 2.23-2.32 (4H, m), 2.38 (4H, t), 3.94-3.99 (2H, d), 4.81-4.95 (1H, m).

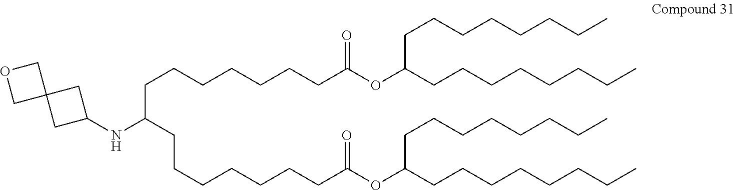 [SMILES: CCCCCCCCC(CCCCCCCC)OC(=O)CCCCCCCC(CCCCCCCC(=O)OC(CCCCCCCC)CCCCCCCC)NC1CC2(COC2)C1]

Sodium triacetoxyborohydride (0.038 g, 0.18 mmol) was added in one portion to a stirred solution of 2-oxaspiro[3.3]heptan-6-amine hydrochloride (0.027 g, 0.18 mmol) and di(heptadecan-9-yl) 9-((2-oxaspiro[3.3]heptan-6-yl)amino)heptadecanedioate (0.026 g, 48.1%) in DCE (4 mL) and NMP (1 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford di(heptadecan-9-yl) 9-((2-oxaspiro[3.3]heptan-6-yl)amino)heptadecanedioate (0.026 g, 48.1%) as a colorless oils. ¹H NMR (500 MHz, METHANOL-d4, 22° C.) δ ppm 0.91 (12H, s), 1.20-1.70 (80H, m), 1.97-2.07 (2H, m), 2.31 (4H, s), 2.51-2.61 (3H, m), 3.03-3.20 (1H, m), 4.59 (2H, s), 4.69-4.76 (2H, s), 4.87 (2H, m); C57H109NO5 m/z calcd. 887.831 observed 889.0 [M+H]+ (LCMS).

### Example 32. Compound 32: 1-(decan-2-yl) 17-(heptadecan-9-yl) 9-((2-oxaspiro[3.3]heptan-6-yl)amino)heptadecanedioate

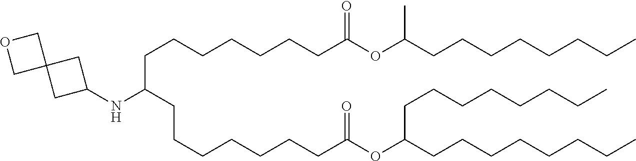 [SMILES: CCCCCCCCC(C)OC(=O)CCCCCCCC(CCCCCCCC(=O)OC(CCCCCCCC)CCCCCCCC)NC1CC2(COC2)C1]

Compound 32 was prepared according to the protocol described for Compound 31 using 17-(heptadecan-9-yloxy)-9,17-dioxoheptadecanoic acid and 1-(decan-2-yl) 15-(2-heptylnonyl) 8-oxopentadecanedioate with the following additional steps. Sodium triacetoxyborohydride (0.040 g, 0.19 mmol) was added in one portion to a stirred solution of 2-oxaspiro[3.3]heptan-6-amine hydrochloride (0.028 g, 0.19 mmol) and 1-(decan-2-yl) 17-(heptadecan-9-yl) 9-oxoheptadecanedioate (0.044 g, 0.06 mmol) in DCE (4 mL) and NMP (1 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford 1-(decan-2-yl) 17-(heptadecan-9-yl) 9-((2-oxaspiro[3.3]heptan-6-yl)amino)heptadecanedioate (0.032 g, 63.4%) as a colorless oils. ¹H NMR (500 MHz, METHANOL-d4, 27° C.) δ ppm 0.92 (9H, t), 1.17-1.70 (71H, m), 1.95-2.07 (2H, m), 2.25-2.39 (4H, m), 2.50-2.62 (3H, m), 3.20-3.29 (1H, m), 4.61 (2H, s), 4.74 (2H, s), 4.87-4.95 (2H, m); C57H109NO5 m/z calcd. 789.721 observed 790.7 [M+H]+ (LCMS).

Scheme 15 below illustrates the synthetic procedures for preparing Example 33.

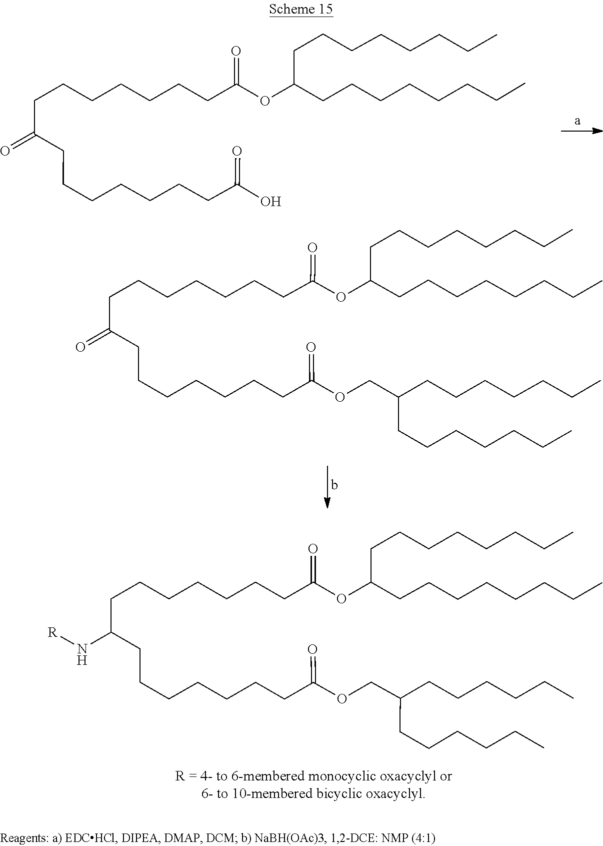

### Example 33. Synthesis of Compound 33: 1-(heptadecan-9-yl) 17-(2-heptylnonyl) 9-((2-oxaspiro[3.3]heptan-6-yl)amino)heptadecanedioate

### Step 1: 1-(heptadecan-9-yl) 17-(2-heptylnonyl) 9-oxoheptadecanedioate

EDC (0.036 g, 0.19 mmol) was added in one portion to a stirred solution of 17-(heptadecan-9-yloxy)-9,17-dioxoheptadecanoic acid (0.05 g, 0.09 mmol), 2-heptylnonan-1-ol (0.046 g, 0.19 mmol), DIPEA (0.049 mL, 0.28 mmol), and DMAP (2.210 mg, 0.02 mmol) in DCM (5 mL) at 0° C. under argon. The resulting solution was stirred at room temperature for 16 hours. The reaction mixture was concentrated under reduced pressure to dryness and redissolved in EtOAc (20 mL) and sodium biocarbonate (20 mL). The layers were separated, and the aqueous layer was extracted with (EtOAc) (3×30 mL). The combined organic layers were washed sequentially with 5% citric acid (25 mL) and saturated aqueous NaCl (25 mL). The organic layer was dried over MgSO₄, filtered and concentrated under reduced pressure to dryness to afford crude product. The resulting residue was purified by flash silica chromatography, elution gradient 0 to 60% EtOAc in hexanes. Product fractions were concentrated under reduced pressure to dryness to afford 1-(heptadecan-9-yl) 17-(2-heptylnonyl) 9-oxoheptadecanedioate (0.039 g, 55.8%) as a colorless oil. ¹H NMR (500 MHz, CHLOROFORM-d, 22° C.) δ ppm 0.89 (12H, m), 1.27 (59H, m), 1.46-1.68 (14H, m), 2.24-2.32 (4H, m), 2.34-2.41 (4H, t), 3.91-4.01 (2H, d), 4.80-4.93 (1H, m).

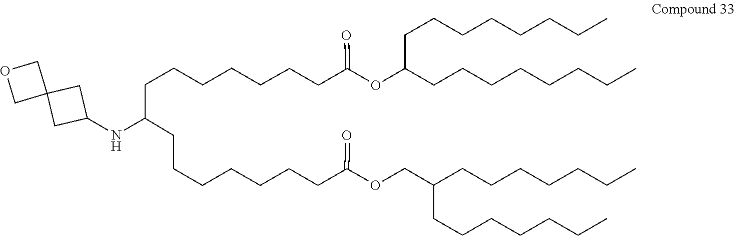 [SMILES: CCCCCCCCC(CCCCCCCC)OC(=O)CCCCCCCC(CCCCCCCC(=O)OCC(CCCCCCC)CCCCCCC)NC1CC2(COC2)C1]

Sodium triacetoxyhydroborate (0.033 g, 0.16 mmol) was added in one portion to a stirred solution of 2-oxaspiro[3.3]heptan-6-amine hydrochloride (0.023 g, 0.15 mmol) and 1-(heptadecan-9-yl) 17-(2-heptylnonyl) 9-oxoheptadecanedioate (0.0392 g, 0.05 mmol) in DCE (4 mL) and NMP (1 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford 1-(heptadecan-9-yl) 17-(2-heptylnonyl) 9-((2-oxaspiro[3.3]heptan-6-yl)amino)heptadecanedioate (0.032 g, 72.3%) as a colorless oil. ¹H NMR (500 MHz, CHLOROFORM-d, 22° C.) δ ppm 0.89 (12H, m), 1.19-1.71 (77H, m), 1.80-1.90 (2H, m), 2.24-2.34 (4H, m), 2.37-2.46 (1H, m), 2.51-2.59 (2H, m), 3.09-3.20 (1H, m), 3.92-4.02 (2H, d), 4.60 (2H, s), 4.71 (2H, s), 4.87 (1H, m); C56H107NO5 m/z calcd. 873.815 observed 875.0 [M+H]+ (LCMS).

Scheme 16 below illustrates the synthetic procedures for preparing Example 34.

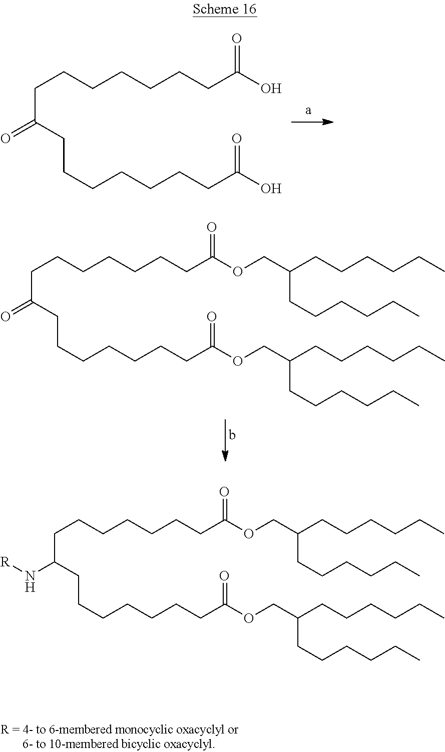

Reagents: a) EDC·HCl, DIPEA, DMAP, DCM; b) NaBH(OAc)3, 1,2-DCE: NMP (4:1)

### Example 34. Synthesis of Compound 34: Bis(2-hexyloctyl) 9-((2-oxaspiro[3.3]heptan-6-yl)amino)heptadecanedioate

### Step 1: bis(2-hexyloctyl) 9-oxoheptadecanedioate

EDC (0.189 g, 0.99 mmol) was added in one portion to a stirred solution of 9-oxoheptadecanedioic acid (0.100 g, 0.32 mmol), 2-hexyloctan-1-ol (0.150 g, 0.70 mmol), DMAP (7.77 mg, 0.06 mmol), and DIPEA (0.228 mL, 1.30 mmol) in MeOH (12 mL) at 25° C. under argon. The resulting solution was stirred at room Temperature for 24 hours. The reaction mixture was diluted with DCM (50 mL) and water (50 mL). The layers were separated, and the aqueous layer was extracted with (DCM) (3×50 mL). The organic layer was dried over MgSO₄, filtered and evaporated to dryness to afford crude product. The resulting residue was purified by flash silica chromatography, elution gradient 0 to 40% EtOAc in hexanes. Product fractions were evaporated to dryness to afford bis(2-hexyloctyl) 9-oxoheptadecanedioate (0.195 g, 87%) as a colorless oil. ¹H NMR (500 MHz, CHLOROFORM-d, 27° C.) δ ppm 0.86 (12H, t), 1.25 (62H, m), 2.21-2.30 (4H, m), 2.35 (4H, s), 3.94 (4H, d).

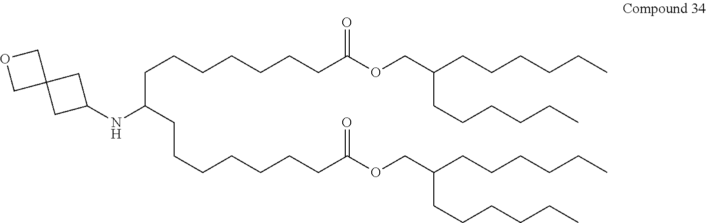 [SMILES: CCCCCCC(CCCCCC)COC(=O)CCCCCCCC(CCCCCCCC(=O)OCC(CCCCCC)CCCCCC)NC1CC2(COC2)C1]

Sodium triacetoxyborohydride (0.040 g, 0.19 mmol) was added in one portion to a stirred solution of 2-oxaspiro[3.3]heptan-6-amine hydrochloride (0.025 g, 0.17 mmol) and bis(2-hexyloctyl) 9-oxoheptadecanedioate (0.050 g, 0.07 mmol) in DCE (2 mL) and NMP (0.5 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford bis(2-hexyloctyl) 9-((2-oxaspiro[3.3]heptan-6-yl)amino)heptadecanedioate (0.034 g, 59.8%) as a colorless oil. ¹H NMR (500 MHz, CHLOROFORM-d, 21° C.) δ ppm 0.82-0.92 (12H, m), 1.17-1.38 (60H, m), 1.56-1.65 (6H, m), 1.79-1.88 (2H, m), 2.29 (4H, t), 2.38-2.45 (1H, m), 2.46-2.59 (2H, m), 3.07-3.18 (1H, m), 3.96 (4H, d), 4.59 (2H, s), 4.70 (2H, c); C51H97NO5 m/z calcd. 803.737 observed 804.80 [M+H]+ (LCMS).

Scheme 17 below illustrates the synthetic procedures for preparing Examples 35 to 37.

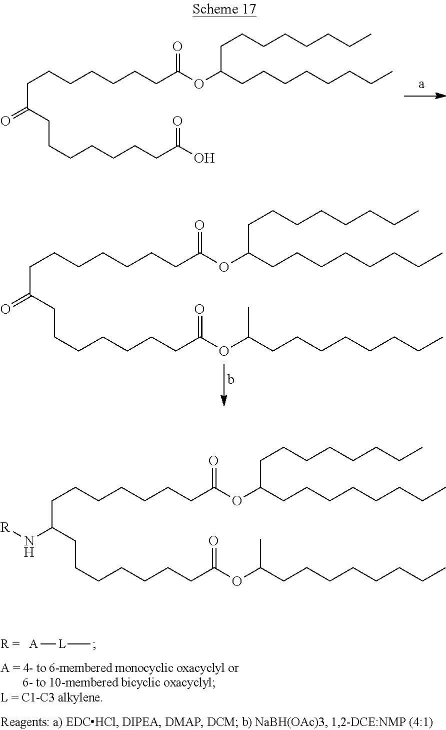

### Example 35. Synthesis of Compound 35: 1-(decan-2-yl) 17-(heptadecan-9-yl) 9-(((tetrahydro-2H-pyran-4-yl)methyl)amino)heptadecanedioate

Step 1: 1-(decan-2-yl) 17-(heptadecan-9-yl) 9-((2-oxaspiro[3.3]heptan-6-yl)amino)heptadecanedioate

3-(((ethylimino)methylene)amino)-N,N-dimethylpropan-1-amine hydrochloride (117 mg, 0.61 mmol) was added in one portion to a stirred mixture of 17-(heptadecan-9-yloxy)-9,17-dioxoheptadecanoic acid (160 mg, 0.29 mmol), decan-2-ol (0.083 mL, 0.43 mmol), DIPEA (0.207 mL, 1.19 mmol), and N,N-dimethylpyridin-4-amine (7.07 mg, 0.06 mmol) in DCM (5 mL) at 0° C. under argon. The resulting mixture was stirred at room temperature for 16 hours. The reaction mixture was diluted with sodium bicarbonate (25 mL) and DCM (25 mL). The layers were separated, and the aqueous layer was extracted with (DCM) (4×25 mL). The combined organic layers were dried over MgSO₄, filtered and concentrated under reduced pressure to dryness to afford crude product. The resulting residue was purified by flash silica chromatography, elution gradient 0 to 40% EtOAc in hexanes. Product fractions were concentrated under reduced pressure to dryness to afford 1-(decan-2-yl) 17-(heptadecan-9-yl) 9-oxoheptadecanedioate (91 mg, 45.4%) as a colorless oil. ¹H NMR (500 MHz, CHLOROFORM-d, 27° C.) δ ppm 0.89 (9H, t), 1.15-1.70 (65H, m), 2.27 (4H, m), 2.35-2.42 (4H, t), 4.80-4.96 (2H, m).

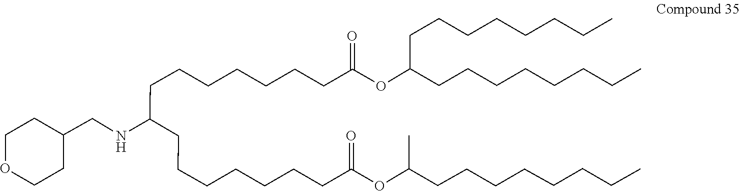 [SMILES: CCCCCCCCC(C)OC(=O)CCCCCCCC(CCCCCCCC(=O)OC(CCCCCCCC)CCCCCCCC)NCC1CCOCC1]

Sodium triacetoxyborohydride (0.038 g, 0.18 mmol) was added in one portion to a stirred solution of (tetrahydro-2H-pyran-4-yl)methanamine hydrochloride (0.024 g, 0.16 mmol) and 1-(decan-2-yl) 17-(heptadecan-9-yl) 9-oxoheptadecanedioate (0.04 g, 0.06 mmol) in DCE (2 mL) and NMP (0.5 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford 1-(decan-2-yl) 17-(heptadecan-9-yl) 9-(((tetrahydro-2H-pyran-4-yl)methyl)amino)heptadecanedioate (0.034 g, 74.1%) as a colorless oil. ¹H NMR (500 MHz, CHLOROFORM-d, 27° C.) δ ppm 0.89 (9H, t), 1.17-1.79 (74H, m), 2.20-2.35 (4H, m), 2.40-2.58 (3H, m), 3.32-3.46 (2H, t), 3.92-4.02 (2H, m), 4.82-4.95 (2H, m); C50H97NO5 m/z calcd. 791.737 observed 792.7 [M+H]+ (LCMS).

### Example 36. Compound 36: 1-(decan-2-yl) 17-(heptadecan-9-yl) 9-(((tetrahydrofuran-3-yl)methyl)amino)heptadecanedioate

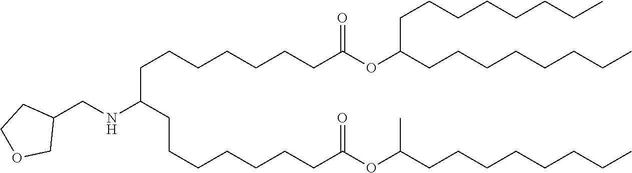 [SMILES: CCCCCCCCC(C)OC(=O)CCCCCCCC(CCCCCCCC(=O)OC(CCCCCCCC)CCCCCCCC)NCC1CCOC1]

Compound 36 was prepared according to the protocol described for Compound 35 using 1-(decan-2-yl) 17-(heptadecan-9-yl) 9-oxoheptadecanedioate with the following additional steps. Sodium triacetoxyborohydride (24.77 mg, 0.12 mmol) was added in one portion to a stirred solution of (tetrahydrofuran-3-yl)methanamine hydrochloride (14.29 mg, 0.10 mmol) and 1-(decan-2-yl) 17-(heptadecan-9-yl) 9-oxoheptadecanedioate (30 mg, 0.04 mmol) in DCE (2 mL) and NMP (0.5 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford 1-(decan-2-yl) 17-(heptadecan-9-yl) 9-(((tetrahydrofuran-3-yl)methyl)amino)heptadecanedioate (26.5 mg, 79%) as a colorless oil. ¹H NMR (500 MHz, METHANOL-d4, 27° C.) δ ppm 0.90 (9H, t), 1.17-1.67 (70H, m), 2.02-2.18 (1H, m), 2.22-2.35 (4H, m), 2.35-2.46 (1H, m), 2.51-2.59 (1H, m), 2.60-2.67 (2H, d), 3.43-3.51 (1H, m), 3.68-3.78 (1H, m), 3.81-3.92 (2H, m), 4.85-4.93 (2H, m); C49H95NO5 m/z calcd. 777.721 observed 778.8 [M+H]+ (LCMS).

### Example 37. Compound 37: 1-(decan-2-yl) 17-(heptadecan-9-yl) 9-((oxetan-3-ylmethyl)amino)heptadecanedioate

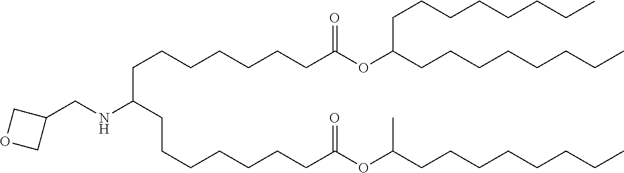 [SMILES: CCCCCCCCC(C)OC(=O)CCCCCCCC(CCCCCCCC(=O)OC(CCCCCCCC)CCCCCCCC)NCC1COC1]

Compound 37 was prepared according to the protocol described for Compound 35 using 1-(decan-2-yl) 17-(heptadecan-9-yl) 9-oxoheptadecanedioate with the following additional steps. Sodium triacetoxyborohydride (24.77 mg, 0.12 mmol) was added in one portion to a stirred solution of oxetan-3-ylmethanamine hydrochloride (12.84 mg, 0.10 mmol) and 1-(decan-2-yl) 17-(heptadecan-9-yl) 9-oxoheptadecanedioate (30 mg, 0.04 mmol) in DCE (2 mL) and NMP (0.5 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford 1-(decan-2-yl) 17-(heptadecan-9-yl) 9-((oxetan-3-ylmethyl)amino)heptadecanedioate (19.80 mg, 59.9%) as a colorless oil. ¹H NMR (500 MHz, METHANOL-d4, 27° C.) δ ppm 0.90 (9H, t), 1.16-1.68 (69H, m), 2.31 (4H, m), 2.47-2.59 (1H, m), 2.91-2.97 (2H, d), 3.07-3.16 (1H, m), 4.35-4.46 (2H, t), 4.75-4.82 (2H, m), 4.85-4.92 (2H, m); C48H93NO5 m/z calcd. 763.705 observed 764.7 [M+H]+ (LCMS).

Scheme 18 below illustrates the synthetic procedures for preparing Examples 38 to 41.

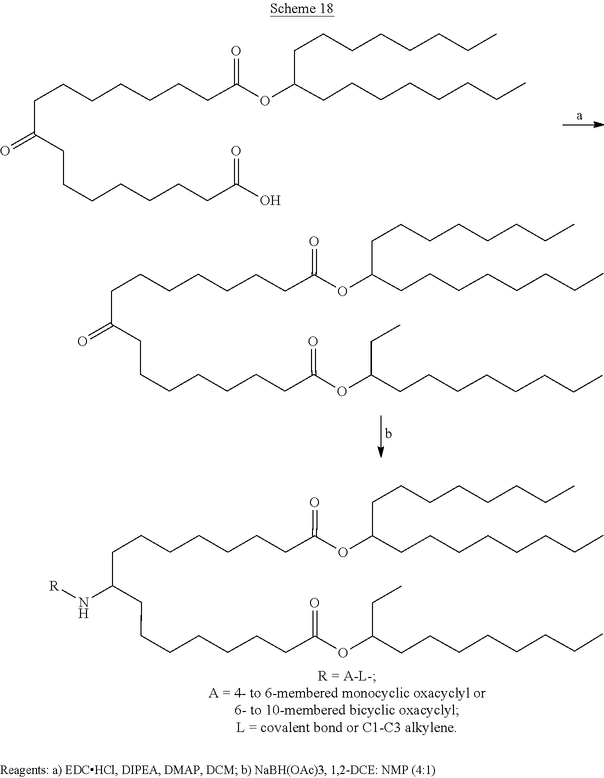

### Example 38. Synthesis of Compound 38: 1-(heptadecan-9-yl) 17-(undecan-3-yl) 9-((2-oxaspiro[3.3]heptan-6-yl)amino)heptadecanedioate

### Step 1: 1-(heptadecan-9-yl) 17-(undecan-3-yl) 9-oxoheptadecanedioate

EDC (0.146 g, 0.76 mmol) was added in one portion to a stirred mixture of 17-(heptadecan-9-yloxy)-9,17-dioxoheptadecanoic acid (0.2 g, 0.36 mmol), undecan-3-ol (0.093 g, 0.54 mmol), DIPEA (0.195 mL, 1.12 mmol), and DMAP (8.84 mg, 0.07 mmol) in DCM (5 mL) at 0° C. under argon. The resulting mixture was stirred at room temperature for 16 hours. The reaction mixture was diluted with 10% citric acid (25 mL) and DCM (25 mL). The layers were separated, and the aqueous layer was extracted with (DCM) (4×25 mL). The combined organic layers were dried over MgSO₄, filtered and concentrated under reduced pressure to dryness to afford crude product. The resulting residue was purified by flash silica chromatography, elution gradient 0 to 40% EtOAc in hexanes. Product fractions were concentrated under reduced pressure to dryness to afford 1-(heptadecan-9-yl) 17-(undecan-3-yl) 9-oxoheptadecanedioate (0.180 g, 70.4%) as a colorless oil.

¹H NMR (500 MHz, CHLOROFORM-d, 27° C.) δ ppm 0.89 (9H, t), 1.16-1.71 (67H, m), 2.23-2.30 (4H, m), 2.38 (4H, t), 4.82-4.95 (2H, m).

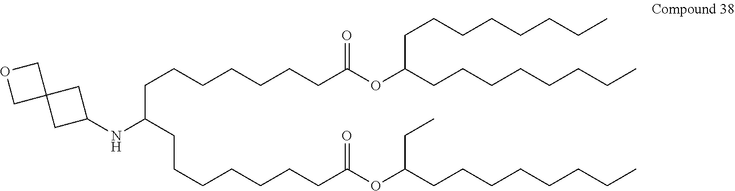 [SMILES: CCCCCCCCC(CC)OC(=O)CCCCCCCC(CCCCCCCC(=O)OC(CCCCCCCC)CCCCCCCC)NC1CC2(COC2)C1]

Sodium triacetoxyborohydride (0.037 g, 0.18 mmol) was added in one portion to a stirred solution of 2-oxaspiro[3.3]heptan-6-amine hydrochloride (0.023 g, 0.15 mmol) and 1-(heptadecan-9-yl) 17-(undecan-3-yl) 9-oxoheptadecanedioate (0.04 g, 0.06 mmol) in DCE (2 mL) and NMP (0.5 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford 1-(heptadecan-9-yl) 17-(undecan-3-yl) 9-((2-oxaspiro[3.3]heptan-6-yl)amino)heptadecanedioate (0.027 g, 59.3%) as a colorless oil. ¹H NMR (500 MHz, METHANOL-d4, 27° C.) δ ppm 0.87-0.96 (12H, m), 1.26-1.70 (68H, m), 1.95-2.06 (2H, m), 2.29-2.37 (4H, m), 2.49-2.60 (3H, m), 3.18-3.29 (1H, m), 4.58-4.63 (2H, s), 4.73 (2H, s), 4.80-4.84 (1H, m), 4.87-4.94 (1H, m); C51H97NO5 m/z calcd. 803.737 observed 804.72 [M+H]+ (LCMS).

### Example 39. Synthesis of Compound 39: 1-(decan-2-yl) 17-(heptadecan-9-yl) 9-((oxetan-3-ylmethyl)amino)heptadecanedioate

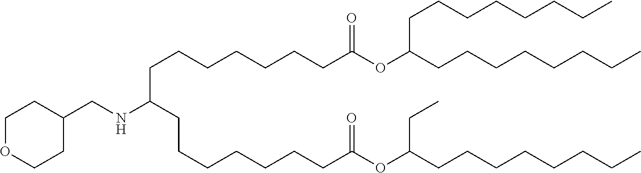 [SMILES: CCCCCCCCC(CC)OC(=O)CCCCCCCC(CCCCCCCC(=O)OC(CCCCCCCC)CCCCCCCC)NCC1CCOCC1]

Compound 39 was prepared according to the protocol described for Compound 38 using 1-(heptadecan-9-yl) 17-(undecan-3-yl) 9-oxoheptadecanedioate with the following additional steps. Sodium triacetoxyborohydride (0.028 g, 0.13 mmol) was added in one portion to a stirred solution of (tetrahydro-2H-pyran-4-yl)methanamine hydrochloride (0.017 g, 0.11 mmol) and 1-(heptadecan-9-yl) 17-(undecan-3-yl) 9-oxoheptadecanedioate (0.03 g, 0.04 mmol) in DCE (2 mL) and NMP (0.5 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford 1-(heptadecan-9-yl) 17-(undecan-3-yl) 9-(((tetrahydro-2H-pyran-4-yl)methyl)amino)heptadecanedioate (0.023 g, 67.8%) as a colorless oil. ¹H NMR (500 MHz, METHANOL-d4, 27° C.) δ ppm 0.84-0.97 (12H, m), 1.22-1.83 (73H, m), 2.26-2.37 (4H, m), 2.55-2.60 (2H, d), 2.62-2.71 (1H, m), 3.36-3.48 (2H, t), 3.90-3.98 (2H, m), 4.77-4.82 (1H, m), 4.85-4.93 (1H, m); C51H99NO5 m/z calcd. 805.752 observed 806.8 [M+H]+(LCMS).

### Example 40. Synthesis of Compound 40: 1-(heptadecan-9-yl) 17-(undecan-3-yl) 9-(((tetrahydrofuran-3-yl)methyl)amino)heptadecanedioate

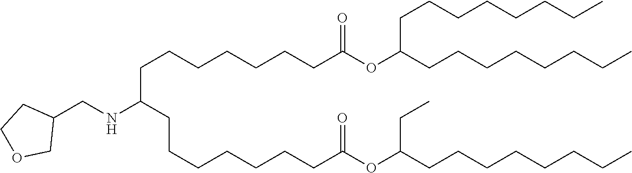 [SMILES: CCCCCCCCC(CC)OC(=O)CCCCCCCC(CCCCCCCC(=O)OC(CCCCCCCC)CCCCCCCC)NCC1CCOC1]

Compound 40 was prepared according to the protocol described for Compound 38 using 1-(heptadecan-9-yl) 17-(undecan-3-yl) 9-oxoheptadecanedioate with the following additional steps. Sodium triacetoxyborohydride (32.4 mg, 0.15 mmol) was added in one portion to a stirred solution of (tetrahydrofuran-3-yl)methanamine hydrochloride (18.68 mg, 0.14 mmol) and 1-(heptadecan-9-yl) 17-(undecan-3-yl) 9-oxoheptadecanedioate (40 mg, 0.06 mmol) in DCE (2 mL) and NMP (0.5 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford 1-(heptadecan-9-yl) 17-(undecan-3-yl) 9-(((tetrahydrofuran-3-yl)methyl)amino)heptadecanedioate (28.7 mg, 64.0%) as a colorless oil. ¹H NMR (500 MHz, METHANOL-d4, 27° C.) δ ppm 0.84-0.97 (12H, m), 1.22-1.69 (69H, m), 2.05-2.16 (1H, m), 2.31 (4H, br d), 2.42 (1H, s), 2.59-2.65 (1H, m), 2.65-2.72 (2H, d), 3.44-3.52 (1H, m), 3.69-3.77 (1H, m), 3.81-3.92 (2H, m), 4.74-4.82 (1H, m), 4.86-4.93 (1H, m); C50H97NO5 m/z calcd. 791.737 observed 792.8 [M+H]+ (LCMS).

### Example 41. Synthesis of Compound 41: 1-(heptadecan-9-yl) 17-(undecan-3-yl) 9-((oxetan-3-ylmethyl)amino)heptadecanedioate

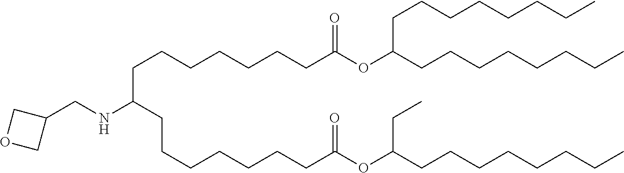 [SMILES: CCCCCCCCC(CC)OC(=O)CCCCCCCC(CCCCCCCC(=O)OC(CCCCCCCC)CCCCCCCC)NCC1COC1]

Compound 41 was prepared according to the protocol described for Compound 38 using 1-(heptadecan-9-yl) 17-(undecan-3-yl) 9-oxoheptadecanedioate with the following additional steps. Sodium triacetoxyborohydride (24.28 mg, 0.11 mmol) was added in one portion to a stirred solution of oxetan-3-ylmethanamine hydrochloride (12.58 mg, 0.10 mmol) and 1-(heptadecan-9-yl) 17-(undecan-3-yl) 9-oxoheptadecanedioate (30 mg, 0.04 mmol) in DCE (2 mL) and NMP (0.5 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford 1-(heptadecan-9-yl) 17-(undecan-3-yl) 9-((oxetan-3-ylmethyl)amino)heptadecanedioate (21.00 mg, 63.6%) as a colorless oil. ¹H NMR (500 MHz, METHANOL-d4, 27° C.) δ ppm 0 0.91 (12H, m), 1.20-1.71 (68H, m), 2.24-2.38 (4H, m), 2.46-2.57 (1H, m), 2.89-2.99 (2H, d), 3.06-3.17 (1H, m), 4.36-4.44 (2H, t), 4.77-4.82 (3H, m), 4.86-4.92 (1H, m); C49H95NO5 m/z calcd. 777.721 observed 778.6 [M+H]+ (LCMS).

Scheme 19 below illustrates the synthetic procedures for preparing Examples 42 and 43.

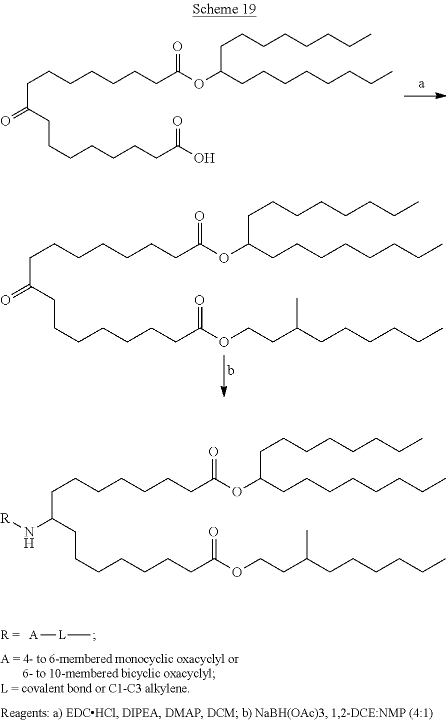

### Example 42. Synthesis of Compound 42: 1-(heptadecan-9-yl) 17-(3-methylnonyl) 9-((2-oxaspiro[3.3]heptan-6-yl)amino)heptadecanedioate

### Step 1: 1-(heptadecan-9-yl) 17-(3-methylnonyl) 9-oxoheptadecanedioate

EDC (58.3 mg, 0.30 mmol) was added in one portion to a stirred mixture of 17-(heptadecan-9-yloxy)-9,17-dioxoheptadecanoic acid (80 mg, 0.14 mmol), 3-methylnonan-1-ol (0.047 mL, 0.22 mmol), DIPEA (0.104 mL, 0.59 mmol), and DMAP (3.54 mg, 0.03 mmol) in DCM (5 mL) at 0° C. under argon. The resulting mixture was stirred at room temperature for 16 hours. The reaction mixture was diluted with DCM (25 mL) and 10% citric acid (25 mL). The layers were separated, and the aqueous layer was extracted with (DCM) (3×25 mL). The combined organic layers were dried over MgSO₄, filtered and concentrated under reduced pressure to dryness to afford crude product. The resulting residue was purified by flash silica chromatography, elution gradient 0 to 60% EtOAc in hexanes. Product fractions were concentrated under reduced pressure to dryness to afford 1-(heptadecan-9-yl) 17-(3-methylnonyl) 9-oxoheptadecanedioate (61.9 mg, 61.7%) as a white solid. ¹H NMR (500 MHz, CHLOROFORM-d, 27° C.) δ ppm 0.82-0.97 (12H, m), 1.10-1.70 (61H, m), 2.25-2.31 (4H, m), 2.38 (4H, t), 4.02-4.18 (2H, m), 4.80-4.93 (1H, m).

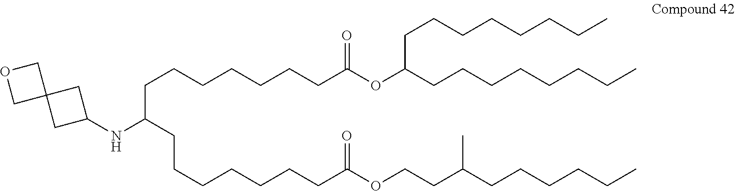 [SMILES: CCCCCCCCC(CCCCCCCC)OC(=O)CCCCCCCC(CCCCCCCC(=O)OCCC(C)CCCCCC)NC1CC2(COC2)C1]

Sodium triacetoxyborohydride (24.77 mg, 0.12 mmol) was added in one portion to a stirred solution of 2-oxaspiro[3.3]heptan-6-amine hydrochloride (15.54 mg, 0.10 mmol) and 1-(heptadecan-9-yl) 17-(3-methylnonyl) 9-oxoheptadecanedioate (30 mg, 0.04 mmol) in DCE (2 mL) and NMP (0.5 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford 1-(heptadecan-9-yl) 17-(3-methylnonyl) 9-((2-oxaspiro[3.3]heptan-6-yl)amino)heptadecanedioate (16.90 mg, 49.4%) as a colorless oil. ¹H NMR (500 MHz, METHANOL-d4, 27° C.) δ ppm 0.85-0.98 (12H, m), 1.13-1.73 (65H, m), 1.96-2.08 (2H, m), 2.31 (4H, t), 2.48-2.59 (3H, m), 3.18-3.27 (1H, m), 4.04-4.19 (2H, m), 4.59 (2H, s), 4.72 (2H, s), 4.85-4.92 (1H, m); C50H95NO5 m/z calcd. 789.721 observed 790.7 [M+H]+ (LCMS).

### Example 43. Synthesis of Compound 43: 1-(heptadecan-9-yl) 17-(3-methylnonyl) 9-(((tetrahydro-2H-pyran-4-yl)methyl)amino)heptadecanedioate

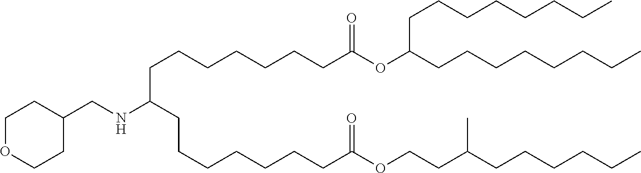 [SMILES: CCCCCCCCC(CCCCCCCC)OC(=O)CCCCCCCC(CCCCCCCC(=O)OCCC(C)CCCCCC)NCC1CCOCC1]

Compound 43 was prepared according to the protocol described for Compound 42 using 1-(heptadecan-9-yl) 17-(3-methylnonyl) 9-oxoheptadecanedioate with the following additional steps. Sodium triacetoxyborohydride (24.77 mg, 0.12 mmol) was added in one portion to a stirred solution of (tetrahydro-2H-pyran-4-yl)methanamine hydrochloride (15.75 mg, 0.10 mmol) and 1-(heptadecan-9-yl) 17-(3-methylnonyl) 9-oxoheptadecanedioate (30 mg, 0.04 mmol) in DCE (2 mL) and NMP (0.5 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford 1-(heptadecan-9-yl) 17-(3-methylnonyl) 9-(((tetrahydro-2H-pyran-4-yl)methyl)amino)heptadecanedioate (9.30 mg, 27.1%) as a colorless oil. ¹H NMR (500 MHz, METHANOL-d4, 27° C.) δ ppm 0.85-0.97 (12H, m), 1.14-1.84 (70H, m), 2.31 (4H, s), 2.56-2.65 (2H, m), 2.63-2.76 (1H, m), 3.36-3.49 (2H, m), 3.88-4.00 (2H, m), 4.04-4.19 (2H, m), 4.85-4.92 (1H, m); C50H97NO5 m/z calcd. 791.737 observed 792.7 [M+H]+ (LCMS).

Scheme 20 below illustrates the synthetic procedures for preparing Example 44.

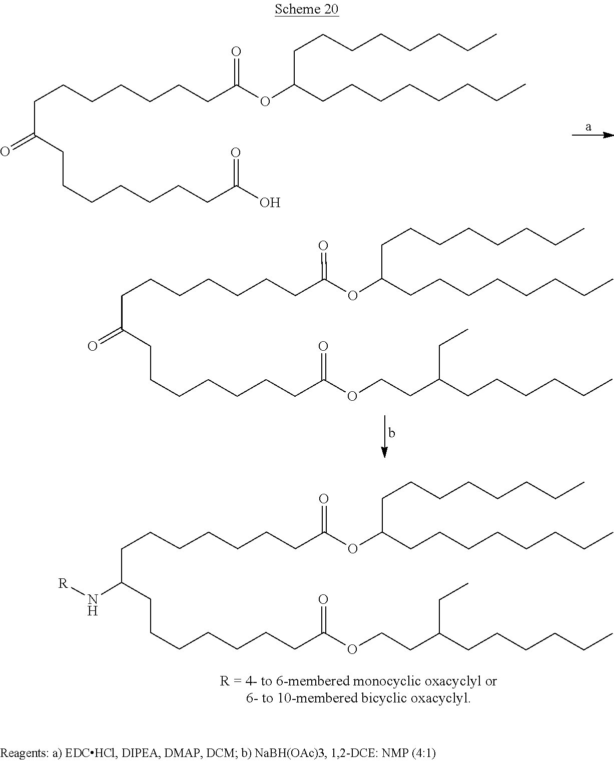

### Example 44. Synthesis of Compound 44: 1-(3-ethylnonyl) 17-(heptadecan-9-yl) 9-((2-oxaspiro[3.3]heptan-6-yl)amino)heptadecanedioate

### Step 1: 1-(3-ethylnonyl) 17-(heptadecan-9-yl) 9-oxoheptadecanedioate

EDC (0.058 g, 0.30 mmol) was added in one portion to a stirred mixture of 17-((3-ethylnonyl)oxy)-9,17-dioxoheptadecanoic acid (0.067 g, 0.14 mmol), heptadecan-9-ol (0.055 g, 0.21 mmol), DIPEA (0.102 mL, 0.59 mmol), and DMAP (3.49 mg, 0.03 mmol) in DCM (5 mL) at 0° C. under argon. The resulting mixture was stirred at room temperature for 16 hours. The reaction mixture was diluted with sodium bicarbonate (25 mL) and DCM (25 mL). The layers were separated, and the aqueous layer was extracted with (DCM) (4×25 mL). The combined organic layers were dried over MgSO₄, filtered and concentrated under reduced pressure to dryness to afford crude product. The resulting residue was purified by flash silica chromatography, elution gradient 0 to 40% EtOAc in hexanes. Product fractions were concentrated under reduced pressure to dryness to afford 1-(3-ethylnonyl) 17-(heptadecan-9-yl) 9-oxoheptadecanedioate (0.040 g, 39.6%) as a colorless oil. ¹H NMR (500 MHz, CHLOROFORM-d, 27° C.) δ ppm 0.81-0.92 (12H, m), 1.17-1.68 (63H, m), 2.21-2.31 (4H, m), 2.34-2.41 (4H, t), 3.98-4.14 (2H, t), 4.79-4.91 (1H, m).

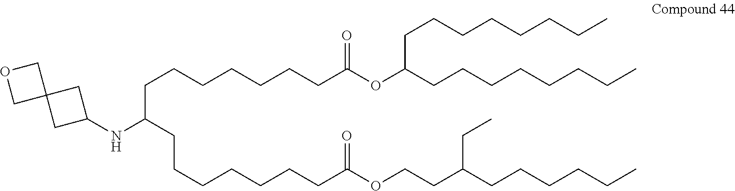 [SMILES: CCCCCCCCC(CCCCCCCC)OC(=O)CCCCCCCC(CCCCCCCC(=O)OCCC(CC)CCCCCC)NC1CC2(COC2)C1]

Sodium triacetoxyborohydride (0.032 g, 0.15 mmol) was added in one portion to a stirred solution of 1-(3-ethylnonyl) 17-(heptadecan-9-yl) 9-oxoheptadecanedioate (0.040 g, 0.06 mmol) and 1-(3-ethylnonyl) 17-(heptadecan-9-yl) 9-oxoheptadecanedioate (0.040 g, 0.06 mmol) in DCE (2 mL) and NMP (0.5 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford 1-(3-ethylnonyl) 17-(heptadecan-9-yl) 9-((2-oxaspiro[3.3]heptan-6-yl)amino)heptadecanedioate (0.029 g, 64.4%) as a colorless oil. ¹H NMR (500 MHz, METHANOL-d4, 27° C.) δ ppm 0.91 (12H, m), 1.22-1.69 (67H, m), 1.96-2.05 (2H, m), 2.31 (4H, t), 2.49-2.59 (3H, m), 3.17-3.27 (1H, m), 4.07-4.15 (2H, t), 4.59 (2H, s), 4.72 (2H, s), 4.84-4.91 (1H, m); C50H95NO5 m/z calcd. 803.737 observed 804.6 [M+H]+(LCMS).

### Example 45. Synthesis of Compound 45: bis(3-pentyloctyl) 7-((tetrahydro-2H-pyran-4-yl)amino)tridecanedioate

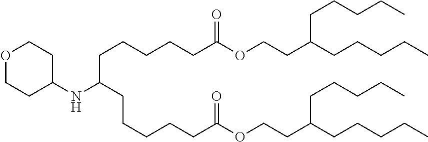 [SMILES: CCCCCC(CCCCC)CCOC(=O)CCCCCC(CCCCCC(=O)OCCC(CCCCC)CCCCC)NC1CCOCC1]

Compound 45 was prepared according to the protocol described for Compound 13 using tetraethyl 6-oxoundecane-1,5,7,11-tetracarboxylate, 7-oxotridecanedioic acid and bis(3-pentyloctyl) 7-oxotridecanedioate with the following additional steps. Sodium triacetoxyhydroborate (44.5 mg, 0.21 mmol) was added in one portion to a stirred solution of bis(3-pentyloctyl) 7-oxotridecanedioate (54.5 mg, 0.09 mmol), tetrahydro-2H-pyran-4-amine (19.02 μl, 0.18 mmol) and acetic acid (262 μl, 0.26 mmol) in DCM (2 mL) and NMP (0.5 mL) at 25° C. under argon. The resulting suspension was stirred at 25° C. for 40 hours. The reaction mixture was diluted with DCM (15 mL), water (5 mL) and sat. Na₂CO₃ (10 mL). The layers were separated, and the aqueous layer was extracted with DCM (3×15 mL). The combined organic layers were dried over MgSO₄, filtered and concentrated under reduced pressure to dryness to afford crude product. The resulting residue was purified by flash silica chromatography, elution gradient 0 to 50% of 20% MeOH/DCM (w/1% NH₄OH) in DCM. Product fractions were concentrated under reduced pressure to dryness to afford bis(3-pentyloctyl) 7-((tetrahydro-2H-pyran-4-yl)amino)tridecanedioate (5.10 mg, 8.23%) as a colorless oil. ¹H NMR (500 MHz, Methanol-d4) δ ppm 0.9 (s, 12H) 1.3 (br s, 48H) 1.6-1.7 (m, 8H) 1.8-1.9 (m, 2H) 2.3 (t, J=7.3 Hz, 4H) 2.7-2.8 (m, 1H) 2.9-3.0 (m, 1H) 3.4 (br d, J=1.7 Hz, 2H) 3.9-4.0 (m, 2H) 4.1 (t, J=6.8 Hz, 4H); C₄₄H₈₅NO₅ m/z calcd. 707.643 observed 708.7 [M+H]⁺ (LCMS).

Scheme 21 below illustrates the synthetic procedures for preparing Example 46. In step w below, AcOH as an additive was used in reductive amination when utilizing the respective amines as a free base.

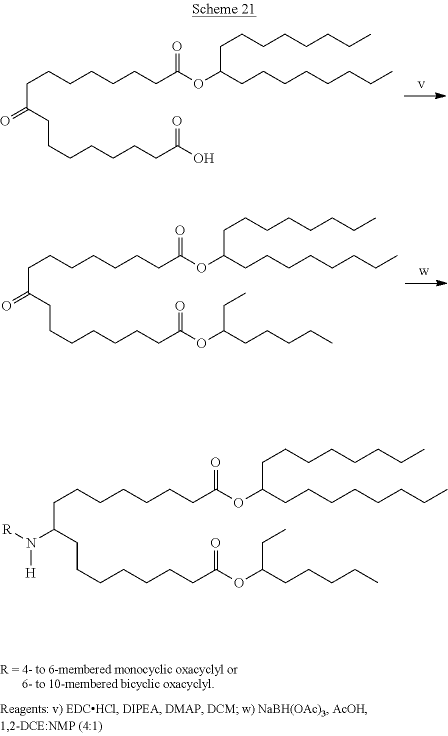

### Example 46. Synthesis of Compound 46: 1-(heptadecan-9-yl) 17-(octan-3-yl) 9-((2-oxaspiro[3.3]heptan-6-yl)amino)heptadecanedioate

Step v): 3-(((ethylimino)methylene)amino)-N,N-dimethylpropan-1-amine hydrochloride (52.0 mg, 0.27 mmol) was added in one portion to a stirred solution of 17-(heptadecan-9-yloxy)-9,17-dioxoheptadecanoic acid (70.7 mg, 0.13 mmol), N-ethyl-N-isopropylpropan-2-amine (0.080 mL, 0.46 mmol), N,N-dimethylpyridin-4-amine (2.343 mg, 0.02 mmol) and octan-3-ol (0.043 mL, 0.27 mmol) in DCM (3 mL) at 0° C. under argon. The resulting solution was stirred at 25° C. for 16 hours. The reaction mixture was diluted with EtOAc (20 mL), water (5 mL) and 5% citric acid solution (15 mL). The layers were separated, and the aqueous layer was extracted with EtOAc (3×20 mL). The combined organic layers were washed with saturated aqueous NaCl (20 mL). The organic layer was dried over MgSO₄, filtered and concentrated under reduced pressure to dryness to afford crude product. The resulting residue was purified by flash silica chromatography, elution gradient 0 to 35% EtOAc in hexanes. Product fractions were concentrated under reduced pressure to dryness to afford 1-(heptadecan-9-yl) 17-(octan-3-yl) 9-oxoheptadecanedioate (52.6 mg, 61.8%) as a colorless oil. ¹H NMR (500 MHz, Chloroform-d) 0.84-0.91 (m, 12H), 1.21-1.33 (m, 42H), 1.46-1.64 (m, 16H), 2.27 (td, J=7.5, 5.3 Hz, 4H), 2.37 (t, J=7.4 Hz, 4H), 4.78-4.89 (m, 2H).

Step w):

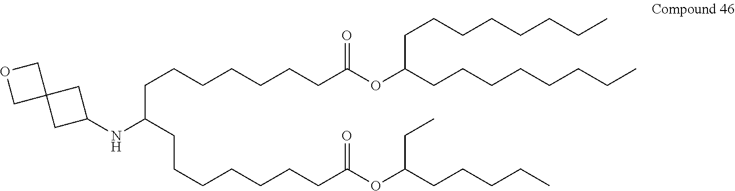 [SMILES: CCCCCCCCC(CCCCCCCC)OC(=O)CCCCCCCC(CCCCCCCC(=O)OC(CC)CCCCC)NC1CC2(COC2)C1]

sodium triacetoxyhydroborate (50.3 mg, 0.24 mmol) was added in one portion (after 10 min) to a stirred solution of 1-(heptadecan-9-yl) 17-(octan-3-yl) 9-oxoheptadecanedioate (52.6 mg, 0.08 mmol), and 2-oxaspiro[3.3]heptan-6-aminium chloride (34.3 mg, 0.23 mmol) in 1,2-DCE (2 mL) and NMP (0.5 mL) under argon. The resulting solution was stirred at 25° C. for 18 hours. The reaction mixture was diluted with DCM (15 mL), water (5 mL) and Sat. Na₂CO₃ (5 mL). The layers were separated, and the aqueous layer was extracted with DCM (3×15 mL). The combined organic layers were dried over MgSO₄, filtered and evaporated to dryness to afford crude product. The resulting residue was purified by flash silica chromatography, elution gradient 0 to 40% of 20% MeOH in DCM (w/1% NH₄OH) in DCM. Product fractions were concentrated under reduced pressure to dryness to afford 1-(heptadecan-9-yl) 17-(octan-3-yl) 9-((2-oxaspiro[3.3]heptan-6-yl)amino)heptadecanedioate (25.9 mg, 43.0%) as a colorless oil. ¹H NMR (400 MHz, Methanol-d4) 0.88-0.95 (m, 12H), 1.26-1.39 (m, 50H), 1.50-1.68 (m, 12H), 1.94-2.02 (m, 2H), 2.33 (td, J=7.2, 2.2 Hz, 4H), 2.46-2.59 (m, 3H), 3.19 (t, J=7.8 Hz, 1H), 4.60 (s, 2H), 4.73 (s, 2H), 4.76-4.83 (m, 1H), 4.88-4.94 (m, 1H); C₄₈H₉₁NO₅ m/z calcd. 761.690 observed 762.6 [M+H]⁺ (LCMS).

Scheme 22 below illustrates the synthetic procedures for preparing Example 47. In step y below, AcOH as an additive was used in reductive amination when utilizing the respective amines as a free base.

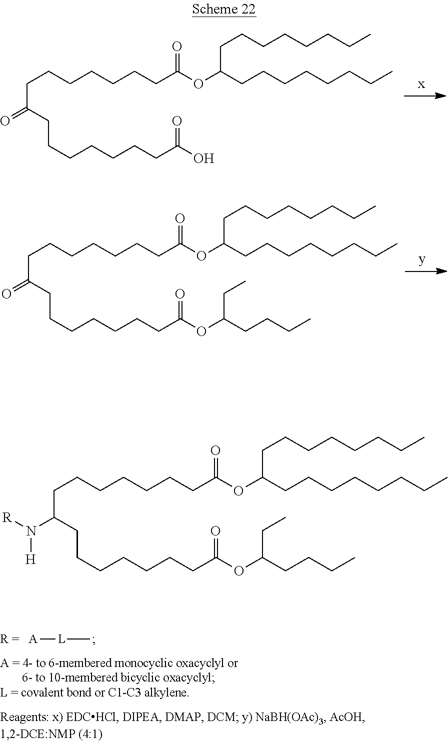

### Example 47. Synthesis of Compound 47: 1-(heptadecan-9-yl) 17-(heptan-3-yl) 9-(((tetrahydrofuran-3-yl)methyl)amino)heptadecanedioate

Step x): 3-(((ethylimino)methylene)amino)-N,N-dimethylpropan-1-amine hydrochloride (52.8 mg, 0.28 mmol) was added in one portion to a stirred solution of 17-(heptadecan-9-yloxy)-9,17-dioxoheptadecanoic acid (63.5 mg, 0.11 mmol), N-ethyl-N-isopropylpropan-2-amine (0.072 mL, 0.41 mmol), N,N-dimethylpyridin-4-amine (2.105 mg, 0.02 mmol) and heptan-3-ol (0.038 mL, 0.26 mmol) in DCM (3 mL) at 0° C. under argon. The resulting solution was stirred at 25° C. for 16 hours. The reaction mixture was diluted with EtOAc (20 mL), water (5 mL) and 5% citric acid solution (15 mL). The layers were separated, and the aqueous layer was extracted with EtOAc (3×20 mL). The combined organic layers were washed with saturated aqueous NaCl (20 mL). The organic layer was dried over MgSO₄, filtered and concentrated under reduced pressure to dryness to afford crude product. The resulting residue was purified by flash silica chromatography, elution gradient 0 to 35% EtOAc in hexanes. Product fractions were concentrated under reduced pressure to dryness to afford 1-(heptadecan-9-yl) 17-(heptan-3-yl) 9-oxoheptadecanedioate (37.0 mg, 49.5%) as a colorless oil. ¹H NMR (500 MHz, Chloroform-d) δ ppm 0.8-0.9 (m, 12H) 1.3 (br s, 40H) 1.5-1.6 (m, 16H) 2.2-2.3 (m, 4H) 2.4 (t, J=7.4 Hz, 4H) 4.8-4.9 (m, 2H).

Step y):

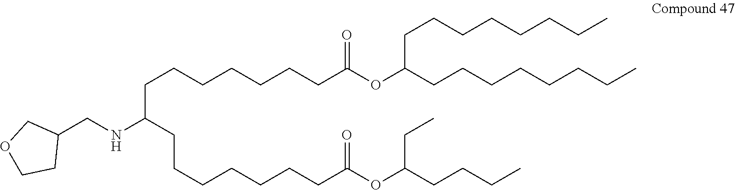 [SMILES: CCCCCCCCC(CCCCCCCC)OC(=O)CCCCCCCC(CCCCCCCC(=O)OC(CC)CCCC)NCC1CCOC1]

sodium triacetoxyhydroborate (33.7 mg, 0.16 mmol) was added in one portion (after 10 min) to a stirred solution of 1-(heptadecan-9-yl) 17-(heptan-3-yl) 9-oxoheptadecanedioate (37 mg, 0.06 mmol), and (tetrahydrofuran-3-yl)methanamine (0.016 mL, 0.15 mmol) in 1,2-DCE (2 mL) and NMP (0.5 mL) under argon. The resulting solution was stirred at 25° C. for 18 hours. The reaction mixture was diluted with DCM (15 mL), water (5 mL) and Sat. Na₂CO₃ (5 mL). The layers were separated, and the aqueous layer was extracted with DCM (3×15 mL). The combined organic layers were dried over MgSO₄, filtered and evaporated to dryness to afford crude product. The resulting residue was purified by flash silica chromatography, elution gradient 0 to 40% of 20% MeOH in DCM (w/1% NH₄OH) in DCM. Product fractions were concentrated under reduced pressure to dryness to afford 1-(heptadecan-9-yl) 17-(heptan-3-yl) 9-(((tetrahydrofuran-3-yl)methyl)amino)heptadecanedioate (29.8 mg, 71.2%) as a colorless oil. ¹H NMR (500 MHz, Methanol-d4) δ ppm 0.9-0.9 (m, 12H) 1.3 (br s, 44H) 1.4-1.4 (m, 4H) 1.5 (br s, 13H) 2.0-2.1 (m, 1H) 2.3 (td, J=7.1, 2.7 Hz, 4H) 2.3-2.4 (m, 1H) 2.5 (br t, J=5.7 Hz, 1H) 2.6 (br d, J=7.2 Hz, 2H) 3.4-3.5 (m, 1H) 3.7 (q, J=7.7 Hz, 1H) 3.8-3.9 (m, 2H) 4.8-4.8 (m, 1H) 4.9-4.9 (m, 1H); C₄₆H₈₉NO₅ m/z calcd. 735.674 observed 736.8 [M+H]⁺ (LCMS).

Scheme 23 below illustrates the synthetic procedures for preparing Example 48. In step aa below, AcOH as an additive was used in reductive amination when utilizing the respective amines as a free base.

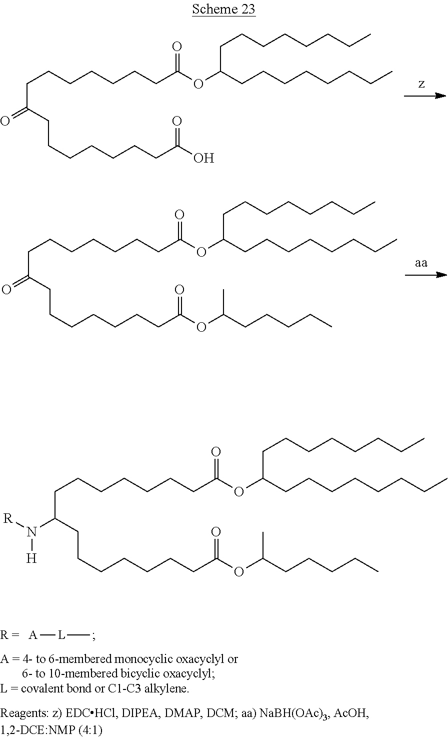

### Example 48. Synthesis of Compound 48: 1-(heptadecan-9-yl) 17-(heptan-2-yl) 9-(((tetrahydrofuran-3-yl)methyl)amino)heptadecanedioate

Step z): 3-(((ethylimino)methylene)amino)-N,N-dimethylpropan-1-amine hydrochloride (58.5 mg, 0.31 mmol) was added in one portion to a stirred solution of 17-(heptadecan-9-yloxy)-9,17-dioxoheptadecanoic acid (64.9 mg, 0.12 mmol), N-ethyl-N-isopropylpropan-2-amine (0.074 mL, 0.42 mmol), N,N-dimethylpyridin-4-amine (2.151 mg, 0.02 mmol) and heptan-2-ol (0.042 mL, 0.29 mmol) in DCM (3 mL) at 0° C. under argon. The resulting solution was stirred at 25° C. for 16 hours. The reaction mixture was diluted with EtOAc (20 mL), water (5 mL) and sat. NaCl solution (15 mL). The layers were separated, and the aqueous layer was extracted with EtOAc (3×20 mL). The combined organic layers were washed with saturated aqueous NaCl (20 mL). The organic layer was dried over MgSO₄, filtered and concentrated under reduced pressure to dryness to afford crude product. The resulting residue was purified by flash silica chromatography, elution gradient 0 to 35% EtOAc in hexanes. Product fractions were concentrated under reduced pressure to dryness to afford 1-(heptadecan-9-yl) 17-(heptan-2-yl) 9-oxoheptadecanedioate (64.0 mg, 84%) as a colorless oil. ¹H NMR (500 MHz, Chloroform-d) δ ppm 0.8-0.9 (m, 9H) 1.2-1.2 (m, 3H) 1.2-1.3 (m, 42H) 1.5-1.6 (m, 14H) 2.2-2.3 (m, 4H) 2.4 (t, J=7.5 Hz, 4H) 4.9 (td, J=13.1, 6.3 Hz, 2H).

Step aa):

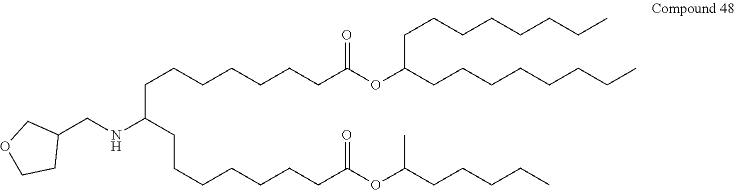 [SMILES: CCCCCCCCC(CCCCCCCC)OC(=O)CCCCCCCC(CCCCCCCC(=O)OC(C)CCCCC)NCC1CCOC1]

sodium triacetoxyhydroborate (58.3 mg, 0.28 mmol) was added in one portion (after 10 min) to a stirred solution of 1-(heptadecan-9-yl) 17-(heptan-2-yl) 9-oxoheptadecanedioate (64 mg, 0.10 mmol), and (tetrahydrofuran-3-yl)methanamine (0.027 mL, 0.27 mmol) in 1,2-DCE (2 mL) and NMP (0.5 mL) under argon. The resulting solution was stirred at 25° C. for 18 hours. The reaction mixture was diluted with DCM (15 mL), water (5 mL) and Sat. Na₂CO₃ (5 mL). The layers were separated, and the aqueous layer was extracted with DCM (3×15 mL). The combined organic layers were dried over MgSO₄, filtered and evaporated to dryness to afford crude product. The resulting residue was purified by flash silica chromatography, elution gradient 0 to 40% of 20% MeOH in DCM (w/1% NH₄OH) in DCM. Product fractions were concentrated under reduced pressure to dryness to afford 1-(heptadecan-9-yl) 17-(heptan-2-yl) 9-(((tetrahydrofuran-3-yl)methyl)amino)heptadecanedioate (52.5 mg, 72.5%) as a colorless oil. ¹H NMR (500 MHz, Methanol-d4) δ ppm 0.9 (s, 9H) 1.2-1.2 (m, 3H) 1.3-1.4 (m, 46H) 1.4-1.5 (m, 4H) 1.5-1.7 (m, 11H) 2.1-2.1 (m, 1H) 2.3-2.3 (m, 4H) 2.4-2.4 (m, 1H) 2.5 (br t, J=5.8 Hz, 1H) 2.6 (d, J=7.2 Hz, 2H) 3.5 (dd, J=8.2, 6.6 Hz, 1H) 3.7 (q, J=7.6 Hz, 1H) 3.8-3.9 (m, 2H) 4.9-4.9 (m, 2H); C₄₆H₈₉NO₅ m/z calcd. 735.674 observed 736.8 [M+H]⁺ (LCMS).

Scheme 24 below illustrates the synthetic procedures for preparing Example 49. In step aj below, AcOH as an additive was used in reductive amination when utilizing the respective amines as a free base.

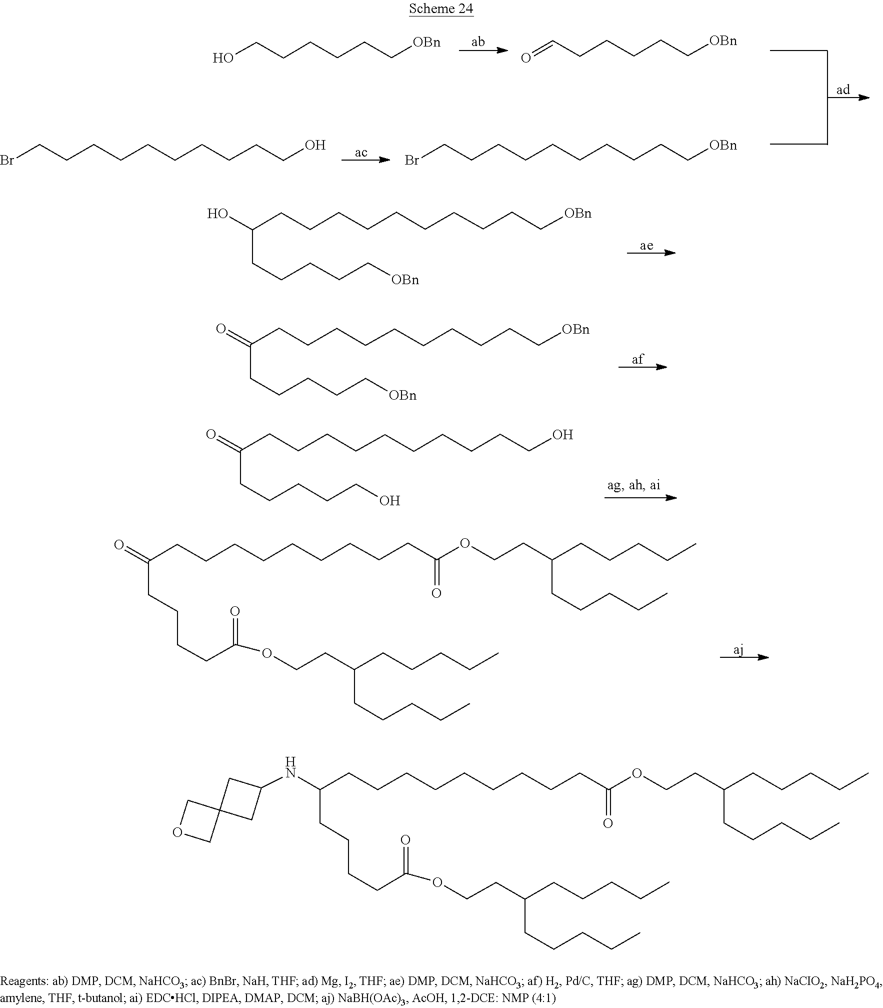

### Example 49. Synthesis of Compound 49: bis(3-pentyloctyl) 6-((2-oxaspiro[3.3]heptan-6-yl)amino)hexadecanedioate

Step ab): Dess-Martin periodinane (2810 mg, 6.63 mmol) was added in one portion to a stirred suspension of sodium bicarbonate (1670 mg, 19.88 mmol) and 6-(benzyloxy)hexan-1-ol (460 mg, 2.21 mmol) in DCM (15 mL) at 0° C. The resulting solution was allowed to come to 25° C. over 5 hours. The reaction mixture was diluted with DCM (20 mL) and washed sequentially with saturated aq. NaHCO₃ (20 mL) and sat. Na₂S₂O₃ (20 mL) The organic layer was dried over MgSO₄, filtered and concentrated under reduced pressure to dryness to afford crude product. The resulting residue was purified by flash silica chromatography, elution gradient 0 to 30% EtOAc in hexanes. Product fractions were concentrated under reduced pressure to dryness to afford 6-(benzyloxy)hexanal (229 mg, 50.2%) as a colorless liquid. ¹H NMR (500 MHz, Chloroform-d) 1.40-1.49 (m, 2H), 1.66 (br d, J=7.6 Hz, 4H), 2.42-2.49 (m, 2H), 3.48 (t, J=6.5 Hz, 2H), 4.51 (s, 2H), 7.28-7.32 (m, 1H), 7.32-7.37 (m, 4H), 9.77 (t, J=1.7 Hz, 1H).

Step ac): sodium hydride (529 mg, 13.22 mmol) was added portion wise to a stirred solution of 10-bromodecan-1-ol (0.872 mL, 4.01 mmol), and (bromomethyl)benzene (0.714 mL, 6.01 mmol) in tetrahydrofuran (9 mL) at 0° C. under argon. The resulting suspension was stirred at 25° C. for 20 hours. The reaction mixture was quenched with saturated aq. NaHCO₃ (10 mL) and diluted with water (10 mL), extracted with DCM (3×20 mL), the organic layer was dried over MgSO₄, filtered and concentrated under reduced pressure to afford crude product. The resulting residue was purified by flash silica chromatography, elution gradient 0 to 30% EtOAc in hexanes. Product fractions were concentrated under reduced pressure to dryness to afford (((10-bromodecyl)oxy)methyl)benzene (1136 mg, 87%) as a colorless oil. ¹H NMR (500 MHz, Chloroform-d) 1.27-1.32 (m, 8H), 1.34-1.45 (m, 4H), 1.58-1.66 (m, 2H), 1.86 (quin, J=7.2 Hz, 2H), 3.39-3.49 (m, 4H), 4.51 (s, 2H), 7.28-7.33 (m, 1H), 7.34-7.36 (m, 4H).

Step ad): iodine (11.63 mg, 0.05 mmol) was added in one portion to a stirred suspension of magnesium (134 mg, 5.50 mmol) and (((10-bromodecyl)oxy)methyl)benzene (600 mg, 1.83 mmol) in THE (5 mL) at 25° C. under argon. The reaction mixture was heated to 55° C. over 0.5 hours. At this point, the color of the reaction mixture changes from brown to cloudy white. 6-(benzyloxy)hexanal (189 mg, 0.92 mmol) was dissolved in 2 mL of THE and added dropwise to the solution at 25° C. Reaction mixture was then warmed up to 60° C. for 2 hours and allowed to come to 25° C. under argon for 15 hours. The reaction mixture was quenched and diluted with water (2 mL), followed by the addition of 1 M HCl (10 mL) and DCM (15 mL). The layers were separated, and the aqueous layer was extracted with DCM (3×15 mL). The combined organic layers were washed with saturated aq. NaCl (15 mL). The organic layer was dried over MgSO₄, filtered and concentrated under reduced pressure to dryness to afford crude product. TLC shows formation of a new less polar product (Rf=0.6; 4:1, hexanes:EtOAc). The resulting residue was purified by flash silica chromatography, elution gradient 0 to 55% hexanes in EtOAc. Product fractions were concentrated under reduced pressure to dryness to afford 1,16-bis(benzyloxy)hexadecan-6-ol (310 mg, 74.4%) as a colorless oil. ¹H NMR (500 MHz, Chloroform-d) 1.28 (br s, 9H), 1.32-1.51 (m, 14H), 1.59-1.69 (m, 4H), 3.48 (td, J=6.5, 4.5 Hz, 4H), 3.58 (br dd, J=7.1, 4.0 Hz, 1H), 4.51 (s, 4H), 7.28-7.31 (m, 2H), 7.33-7.36 (m, 8H).

Step ae): Dess-Martin periodinane (578 mg, 1.36 mmol) was added in one portion to a stirred suspension of sodium bicarbonate (344 mg, 4.09 mmol) and 1,16-bis(benzyloxy)hexadecan-6-ol (310 mg, 0.68 mmol) in DCM (10 mL) at 0° C. The resulting solution was allowed to come to 25° C. over 24 hours. The reaction mixture was diluted with DCM (20 mL) and washed sequentially with saturated aq. NaHCO₃ (20 mL) and sat. Na₂S₂O₃ (20 mL). The organic layer was dried over MgSO₄, filtered and concentrated under reduced pressure to dryness to afford crude product. The resulting residue was purified by flash silica chromatography, elution gradient 0 to 30% EtOAc in hexanes. Product fractions were concentrated under reduced pressure to dryness to afford 1,16-bis(benzyloxy)hexadecan-6-one (270 mg, 87%) as a colorless residue. ¹H NMR (500 MHz, Chloroform-d) 1.20-1.27 (m, 10H), 1.29-1.36 (m, 4H), 1.47-1.61 (m, 8H), 2.27-2.35 (m, 4H), 3.41 (t, J=6.6 Hz, 4H), 4.43 (d, J=4.1 Hz, 4H), 7.17-7.25 (m, 2H), 7.25-7.31 (m, 8H).

Step af): 1,16-bis(benzyloxy)hexadecan-6-one (270 mg, 0.60 mmol), palladium 10% on carbon (127 mg, 0.12 mmol) in THE (6 mL) were stirred under an atmosphere of hydrogen at 25° C. for 16 hours. The reaction mixture was filtered through Celite. The precipitate was obtained by evaporation of the solvent to afford 1,16-dihydroxyhexadecan-6-one (168 mg, 104%) as a white solid***. ¹H NMR (500 MHz, Chloroform-d) 1.29 (br s, 10H), 1.34-1.41 (m, 4H), 1.54-1.70 (m, 10H), 2.41 (dt, J=15.0, 7.4 Hz, 4H), 3.63-3.68 (m, 4H).

Steps ag, ah, ai): Dess-Martin periodinane (785 mg, 1.85 mmol) was added in one portion to a stirred suspension of sodium bicarbonate (466 mg, 5.55 mmol) and 1,16-dihydroxyhexadecan-6-one (168 mg, 0.62 mmol) in DCM (10 mL) at 0° C. The resulting solution was allowed to come to 25° C. over 24 hours. The reaction mixture was diluted with DCM (20 mL) and washed sequentially with saturated aq. NaHCO₃ (20 mL) and sat. Na₂S₂O₃ (20 mL) The organic layer was dried over MgSO₄, filtered and concentrated under reduced pressure to dryness to afford 6-oxohexadecanedial as a colorless dry film, which was used without further purification.

6-oxohexadecanedial (239 mg, 0.89 mmol) was added to a stirred solution of 2-methyl-2-butene (2.83 mL, 26.71 mmol), sodium dihydrogen phosphate (641 mg, 5.34 mmol) and sodium chlorite (5.34 mL, 5.34 mmol) in THE (15 mL) and t-butanol (7.50 mL) at 25° C. The resulting solution was stirred at 25° C. for 4 hours. The reaction mixture was diluted with DCM and Water (30 ml each). The reaction mixture was adjusted to pH=3 with 1 M HCl solution. The organic layer was dried over MgSO₄, filtered and concentrated under reduced pressure to dryness to afford desired product 6-oxohexadecanedioic acid as a white solid, which was used without further purification.

3-(((ethylimino)methylene)amino)-N,N-dimethylpropan-1-amine hydrochloride (392 mg, 2.05 mmol) was added in one portion to a stirred solution of 6-oxohexadecanedioic acid (186.3 mg, 0.62 mmol), 3-pentyloctan-1-ol (373 mg, 1.86 mmol), N-ethyl-N-isopropylpropan-2-amine (0.486 mL, 2.79 mmol) and N,N-dimethylpyridin-4-amine (11.37 mg, 0.09 mmol) in DCM (8 mL) at 0° C. under argon. The resulting solution was stirred at 25° C. for 18 hours. The reaction mixture was diluted with DCM (10 mL) and water (10 mL). The layers were separated, and the aqueous layer was extracted with DCM (3×15 mL). The combined organic layers were washed with saturated aq. NaCl (15 mL). The organic layer was dried over MgSO₄, filtered and concentrated under reduced pressure to dryness to afford crude product. The resulting residue was purified by flash silica chromatography, elution gradient 0 to 30% EtOAc in hexanes. Product fractions were concentrated under reduced pressure to dryness to afford bis(3-pentyloctyl) 6-oxohexadecanedioate (116 mg, 28.2%, 3 steps) as a colorless oil. ¹H NMR (500 MHz, Chloroform-d) 0.88-0.93 (m, 16H), 1.24-1.37 (m, 44H), 1.55-1.66 (m, 8H), 2.28-2.34 (m, 4H), 2.37-2.46 (m, 4H), 4.10 (t, J=7.0 Hz, 4H).

Step aj):

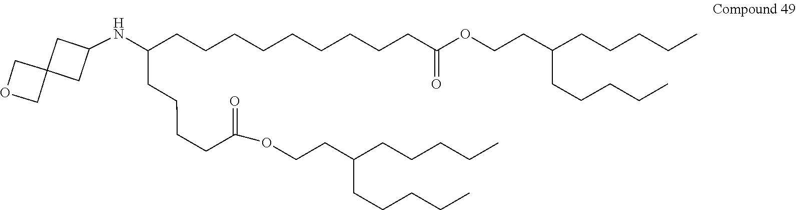 [SMILES: CCCCCC(CCCCC)CCOC(=O)CCCCCCCCCC(CCCCC(=O)OCCC(CCCCC)CCCCC)NC1CC2(COC2)C1]

sodium triacetoxyhydroborate (111 mg, 0.52 mmol) was added in one portion (after 10 min) to a stirred solution of bis(3-pentyloctyl) 6-oxohexadecanedioate (116.3 mg, 0.17 mmol), and 2-oxaspiro[3.3]heptan-6-aminium chloride (76 mg, 0.51 mmol) in 1,2-DCE (2.4 mL) and NMP (0.5 mL) under argon. The resulting solution was stirred at 25° C. for 40 hours. The reaction mixture was diluted with DCM (15 mL), water (5 mL) and Sat. Na₂CO₃ (5 mL). The layers were separated, and the aqueous layer was extracted with DCM (3×15 mL). The combined organic layers were dried over MgSO₄, filtered and evaporated to dryness to afford crude product. The resulting residue was purified by flash silica chromatography, elution gradient 0 to 40% of 20% MeOH in DCM (w/1% NH₄OH) in DCM. Product fractions were concentrated under reduced pressure to dryness to afford bis(3-pentyloctyl) 6-((2-oxaspiro[3.3]heptan-6-yl)amino)hexadecanedioate (18.6 mg, 13.95%) as a colorless oil. ¹H NMR (500 MHz, Methanol-d4) 0.93 (t, J=7.0 Hz, 12H), 1.28-1.67 (m, 60H), 2.11 (ddd, J=12.6, 8.4, 4.0 Hz, 2H), 2.34 (dt, J=18.8, 7.2 Hz, 4H), 2.57-2.65 (m, 2H), 2.65-2.71 (m, 1H), 3.34-3.40 (m, 1H), 4.13 (td, J=6.8, 1.8 Hz, 4H), 4.62 (s, 2H), 4.74 (s, 2H). C₄₈H₉₁NO₅ m/z calcd. 761.690 observed 762.9 [M+H]⁺ (LCMS).

Scheme 25 below illustrates the synthetic procedures for preparing Example 50.

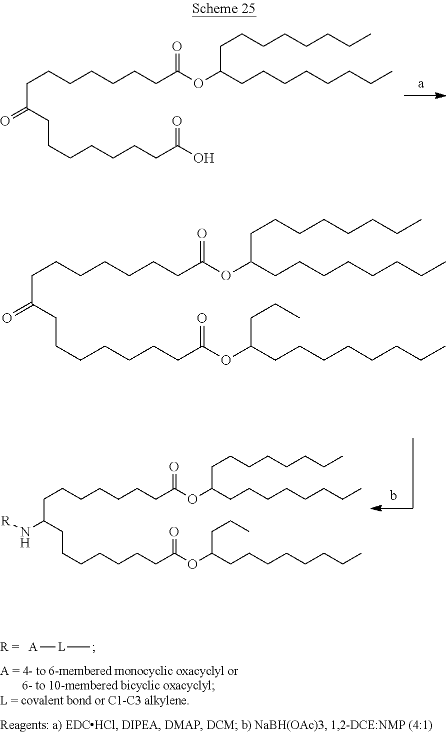

### Example 50. Synthesis of Compound 50

### Step 1: 1-(dodecan-4-yl) 17-(heptadecan-9-yl) 9-oxoheptadecanedioate

3-(((ethylimino)methylene)amino)-N,N-dimethylpropan-1-amine hydrochloride (118 mg, 0.62 mmol) was added in one portion to a stirred solution of 17-(heptadecan-9-yloxy)-9,17-dioxoheptadecanoic acid (262 mg, 0.47 mmol), N-ethyl-N-isopropylpropan-2-amine (0.289 mL, 1.66 mmol), N,N-dimethylpyridin-4-amine (11.58 mg, 0.09 mmol) and dodecan-4-ol (106 mg, 0.57 mmol) in DCM (10 mL) at 0° C. under argon. The resulting solution was stirred at RT for 16 hours. The reaction mixture was diluted with DCM (20 mL) and 10% citric acid solution (25 mL). The organic layer was separated, and the aqueous layer was extracted with (DCM) (3×25 mL). The combined organic layers were washed with saturated aqueous NaCl (20 mL). The organic layer was dried over MgSO₄, filtered and concentrated under reduced pressure to dryness to afford crude product. The resulting residue was purified by flash silica chromatography, elution gradient 0 to 20% EtOAc in hexanes. Product fractions were concentrated under reduced pressure to dryness to afford 1-(dodecan-4-yl) 17-(heptadecan-9-yl) 9-oxoheptadecanedioate (213 mg, 62.3%) as a colorless oil. ¹H NMR (500 MHz, CHLOROFORM-d, 27° C.) δ ppm 0.82-0.98 (12H, m), 1.18-1.68 (66H, m), 2.28 (4H, t), 2.34-2.43 (4H, t), 4.76-4.97 (2H, m).

### Compound 50: 1-(dodecan-4-yl) 17-(heptadecan-9-yl) 9-((2-oxaspiro[3.3]heptan-6-yl)amino)heptadecanedioate

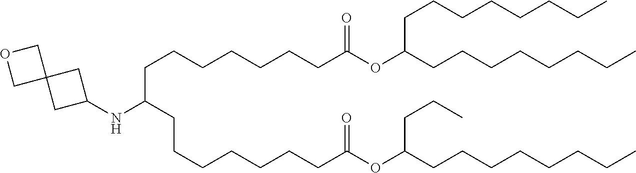 [SMILES: CCCCCCCCC(CCC)OC(=O)CCCCCCCC(CCCCCCCC(=O)OC(CCCCCCCC)CCCCCCCC)NC1CC2(COC2)C1]

Sodium triacetoxyborohydride (23.80 mg, 0.11 mmol) was added in one portion to a stirred solution of 2-oxaspiro[3.3]heptan-6-amine hydrochloride (14.94 mg, 0.10 mmol), and 1-(dodecan-4-yl) 17-(heptadecan-9-yl) 9-oxoheptadecanedioate (30 mg, 0.04 mmol) in DCE (2 mL) and NMP (0.5 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford 1-(dodecan-4-yl) 17-(heptadecan-9-yl) 9-((2-oxaspiro[3.3]heptan-6-yl)amino)heptadecanedioate (10.50 mg, 30.8%) as a colorless oil. ¹H NMR (500 MHz, CHLOROFORM-d, 27° C.) δ ppm 0.91 (12H, m), 1.23-1.68 (70H, m), 1.96-2.08 (2H, m), 2.25-2.36 (4H, t), 2.51-2.60 (3H, m), 3.21-3.28 (1H, m), 4.57-4.62 (2H, s), 4.72 (2H, s), 4.87-4.93 (2H, m); C52H99NO5 m/z calcd. 817.752 observed 818.90 [M+H]+ (LCMS).

Scheme 26 below illustrates the synthetic procedures for preparing Examples 51 and 52.

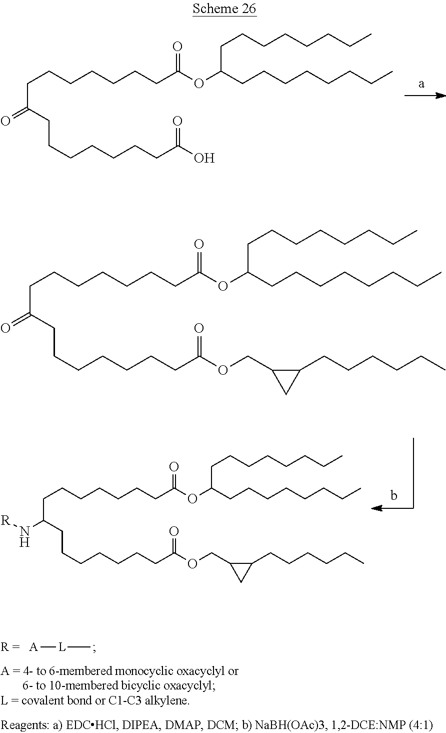

### Examples 51 and 52. Synthesis of Compounds 51 and 52

### Step 1: 1-(heptadecan-9-yl) 17-((2-hexylcyclopropyl)methyl) 9-oxoheptadecanedioate

3-(((ethylimino)methylene)amino)-N,N-dimethylpropan-1-amine hydrochloride (90 mg, 0.47 mmol) was added in one portion to a stirred solution of 17-(heptadecan-9-yloxy)-9,17-dioxoheptadecanoic acid (123 mg, 0.22 mmol), N-ethyl-N-isopropylpropan-2-amine (0.081 mL, 0.47 mmol), N,N-dimethylpyridin-4-amine (5.44 mg, 0.04 mmol) and (2-hexylcyclopropyl)methanol (41.7 mg, 0.27 mmol) in DCM (5 mL) at 0° C. under argon. The resulting solution was stirred at RT for 16 hours. The reaction mixture was diluted with DCM (20 mL) and 10% citric acid solution (25 mL). The organic layer was separated, and the aqueous layer was extracted with (DCM) (3×25 mL). The combined organic layers were washed with saturated aqueous NaCl (20 mL). The organic layer was dried over MgSO₄, filtered and concentrated under reduced pressure to dryness to afford crude product. The resulting residue was purified by flash silica chromatography, elution gradient 0 to 40% EtOAc in hexanes. Product fractions were concentrated under reduced pressure to dryness to afford 1-(heptadecan-9-yl) 17-((2-hexylcyclopropyl)methyl) 9-oxoheptadecanedioate (100 mg, 65.0%) as a colorless oil. ¹H NMR (500 MHz, CHLOROFORM-d, 27° C.) δ ppm −0.02-0.09 (1H, m), 0.70-0.80 (1H, m), 0.84-0.97 (1 OH, m), 1.09-1.70 (59H, m), 2.24-2.46 (8H, m), 3.88-4.27 (2H, m), 4.88 (1H, m).

### Compound 51: 1-(heptadecan-9-yl) 17-((2-hexylcyclopropyl)methyl) 9-((oxetan-3-ylmethyl)amino)heptadecanedioate

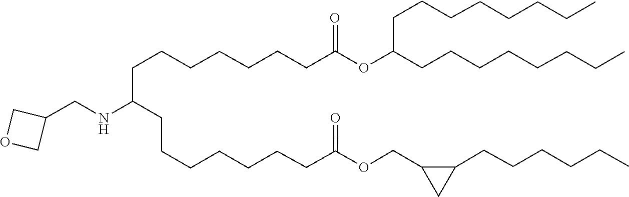 [SMILES: CCCCCCCCC(CCCCCCCC)OC(=O)CCCCCCCC(CCCCCCCC(=O)OCC1CC1CCCCCC)NCC1COC1]

Sodium triacetoxyborohydride (20.70 mg, 0.10 mmol) was added in one portion to a stirred solution of oxetan-3-ylmethanamine hydrochloride (10.73 mg, 0.09 mmol) and 1-(heptadecan-9-yl) 17-((2-hexylcyclopropyl)methyl) 9-oxoheptadecanedioate (25 mg, 0.04 mmol) in DCE (2 mL) and NMP (0.5 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford 1-(heptadecan-9-yl) 17-((2-hexylcyclopropyl)methyl) 9-((oxetan-3-ylmethyl)amino)heptadecanedioate (9.20 mg, 33.4%) as a colorless oil. ¹H NMR (500 MHz, METHANOL-d4, 27° C.) δ ppm 0.01-0.09 (1H, m), 0.72-0.81 (1H, m), 0.88-0.96 (10H, m), 1.12-1.73 (64H, m), 2.33 (4H, q), 2.54-2.63 (1H, m), 3.00 (2H, m), 3.10-3.23 (1H, m), 3.61-3.77 (1H, m), 3.85-3.97 (1H, m), 4.22-4.30 (1H, m), 4.42 (2H, t), 4.89-4.97 (1H, m); C48H91 NO5 m/z calcd. 761.690 observed 762.80 [M+H]+ (LCMS).

### Compound 52: 1-(heptadecan-9-yl) 17-((2-hexylcyclopropyl)methyl) 9-(((tetrahydrofuran-3-yl)methyl)amino)heptadecanedioate

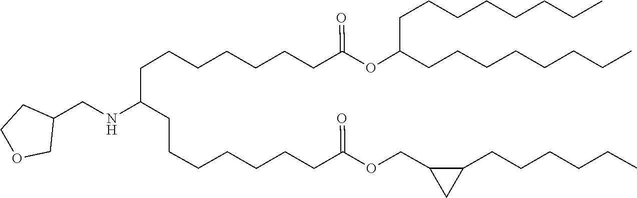 [SMILES: CCCCCCCCC(CCCCCCCC)OC(=O)CCCCCCCC(CCCCCCCC(=O)OCC1CC1CCCCCC)NCC1CCOC1]

Sodium triacetoxyborohydride (20.70 mg, 0.10 mmol) was added in one portion to a stirred solution of (tetrahydrofuran-3-yl)methanamine hydrochloride (11.95 mg, 0.09 mmol) and 1-(heptadecan-9-yl) 17-((2-hexylcyclopropyl)methyl) 9-oxoheptadecanedioate (25 mg, 0.04 mmol) in DCE (2 mL) and NMP (0.5 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford 1-(heptadecan-9-yl) 17-((2-hexylcyclopropyl)methyl) 9-(((tetrahydrofuran-3-yl)methyl)amino)heptadecanedioate (15.60 mg, 55.6%) as a colorless oil. 1H NMR (500 MHz, METHANOL-d4, 270C) δ ppm 1H NMR (500 MHz, METHANOL-d4, 270C) 0.01-0.09 (1H, m), 0.71-0.82 (1H, m), 0.87-0.98 (10H, m), 1.13-1.72 (64H, m), 2.08-2.18 (1H, m), 2.33 (4H, m), 2.41-2.50 (1H, m), 2.63-2.70 (1H, m), 2.70-2.75 (2H, m), 3.47-3.55 (1H, m), 3.70-3.81 (1H, m), 3.89 (3H, m), 4.22-4.30 (1H, m), 4.89-4.93 (1H, m); C49H93NO5 m/z calcd. 775.705 observed 776.90 [M+H]+ (LCMS).

Scheme 27 below illustrates the synthetic procedures for preparing Example 53.

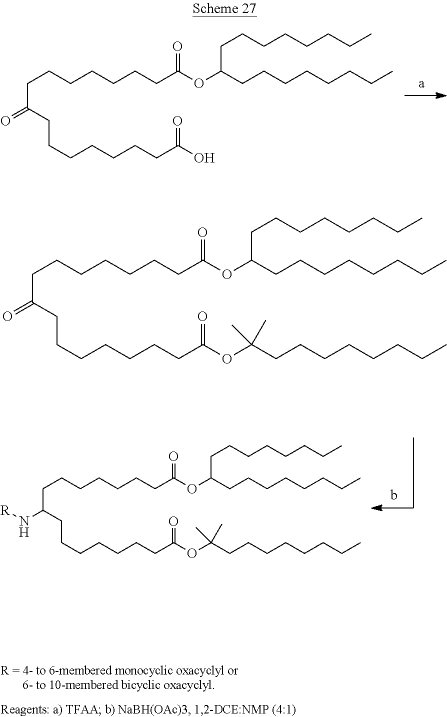

### Example 53. Synthesis of Compound 53

### Step 1: 1-(heptadecan-9-yl) 17-(2-methyldecan-2-yl) 9-oxoheptadecanedioate

To a solution of 17-(heptadecan-9-yloxy)-9,17-dioxoheptadecanoic acid (28 mg, 0.05 mmol) in DCM (2 mL) at 0° C., TFAA (0.016 mL, 0.11 mmol) was added dropwise. After 2.5 h, 2-methyldecan-2-ol (31.4 mg, 0.18 mmol) was slowly added. After 1 h the reaction was warmed to rt and allowed to stir for 2.5 h. The reaction was quenched with water and extracted with diethylether. The organic layer was separated and dried over MgSO₄, filtered and concentrated under reduced pressure to dryness to afford crude product. The residue was purified by silica gel chromatography with (0-10%) EtOAc in hexanes to obtain 1-(heptadecan-9-yl) 17-(2-methyldecan-2-yl) 9-oxoheptadecanedioate (24.50 mg, 68.4%) as a pale yellow oil. ¹H NMR (500 MHz, CHLOROFORM-d, 27° C.) δ ppm 0.85-0.95 (9H, t), 1.22-1.37 (48H, m), 1.43 (6H, s), 1.58 (14H, m), 2.18-2.24 (2H, t), 2.26-2.31 (2H, t), 2.36-2.42 (4H, t), 4.82-4.95 (1H, m).

### Compound 53: 1-(heptadecan-9-yl) 17-(3-methylnonyl) 9-(((tetrahydro-2H-pyran-4-yl)methyl)amino)heptadecanedioate

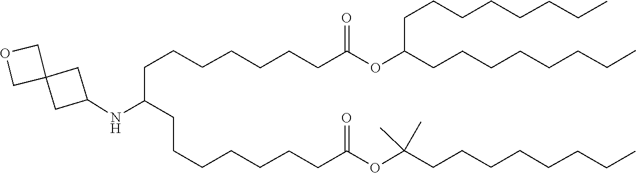 [SMILES: CCCCCCCCC(CCCCCCCC)OC(=O)CCCCCCCC(CCCCCCCC(=O)OC(C)(C)CCCCCCCC)NC1CC2(COC2)C1]

Sodium triacetoxyborohydride (19.83 mg, 0.09 mmol) was added in one portion to a stirred solution of 2-oxaspiro[3.3]heptan-6-amine hydrochloride (12.44 mg, 0.08 mmol) and 1-(heptadecan-9-yl) 17-(2-methyldecan-2-yl) 9-oxoheptadecanedioate (24.5 mg, 0.03 mmol) in DCE (2 mL) and NMP (0.5 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford 1-(heptadecan-9-yl) 17-(2-methyldecan-2-yl) 9-((2-oxaspiro[3.3]heptan-6-yl)amino)heptadecanedioate (18.20 mg, 65.3%) as a colorless oil. ¹H NMR (500 MHz, METHANOL-d4, 27° C.) δ ppm 0.92 (9H, t), 1.25-1.70 (70H, m), 1.73-1.82 (2H, m), 1.96-2.06 (2H, m), 2.20-2.26 (2H, m), 2.30-2.36 (2H, m), 2.49-2.54 (1H, m), 2.54-2.60 (2H, m), 3.18-3.27 (1H, m), 4.60 (2H, s), 4.73 (2H, s), 4.88-4.93 (1H, m); C50H97NO5 m/z calcd. 803.737 observed 803.90 [M+H]+ (LCMS).

Scheme 28 below illustrates the synthetic procedures for preparing Example 54.

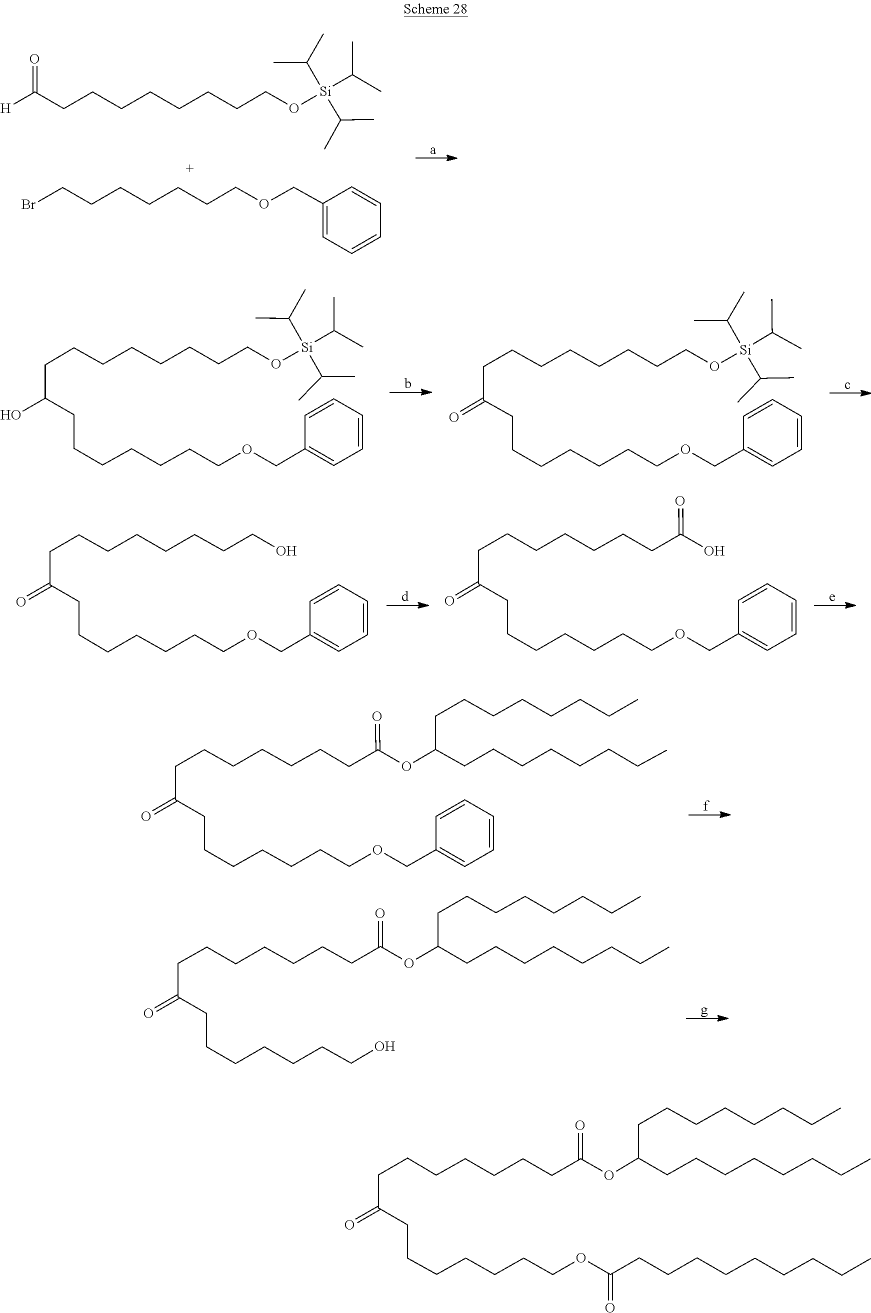

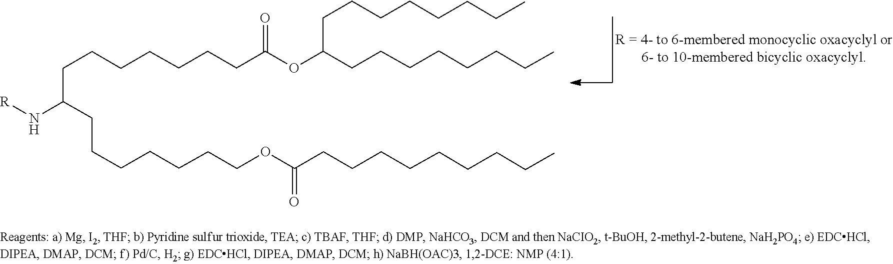

### Example 54. Synthesis of Compound 54

### Step 1: 16-(benzyloxy)-1-((triisopropylsilyl)oxy)hexadecan-8-ol

To a suspension of Mg turnings in THF (15 mL) containing a small iodine crystal were added few drops of the appropriate brominated compound (1 equiv) in THF (0.5 mL/mmol of substrate). The mixture was heated until the reaction started, then (((7-bromoheptyl)oxy)methyl)benzene (0.589 g, 2.07 mmol) was added drop by drop to maintain a non-assisted gentle reflux. After complete addition of the starting material, the mixture was heated under reflux for 1 h. The solution of Grignard reagent was cooled down and titrated prior to use. 9-((triisopropylsilyl)oxy)nonanal (0.500 g, 1.59 mmol) was added in one portion to the stirred mixture under argon. The resulting mixture was stirred at 70° C. for 16 hours. The reaction mixture was quenched with water (50 mL), extracted with DCM (3×25 mL), the organic layer was dried over MgSO₄, filtered and concentrated under reduced pressure to dryness to afford pale yellow oil. The resulting residue was purified by flash silica chromatography, elution gradient 0 to 20% EtOAc in hexanes. Product fractions were concentrated under reduced pressure to dryness to afford 1-(benzyloxy)-16-((triisopropylsilyl)oxy)hexadecan-8-ol (0.392 g, 47.3%) as a pale yellow oil. ¹H NMR (500 MHz, CHLOROFORM-d, 27° C.) δ ppm 1.04-1.14 (21H, m), 1.33 (22H, m), 1.51-1.57 (2H, m), 1.60-1.67 (2H, m), 3.44-3.52 (2H, t), 3.58-3.63 (1H, m), 3.65-3.71 (2H, t), 4.53 (2H, s), 7.29-7.40 (5H, m).

### Step 2: 1-(benzyloxy)-16-((triisopropylsilyl)oxy)hexadecan-8-one

To an oven-dried flask, 1-(benzyloxy)-16-((triisopropylsilyl)oxy)hexadecan-8-ol (0.392 g, 0.75 mmol) was dissolved in DCM (10 mL). DMSO (2.000 mL) was then added, followed by TEA (1.049 mL, 7.53 mmol) to the reaction mixture. The mixture was cool to 0° C. pyridine-sulfur trioxide (1/1) (0.958 g, 6.02 mmol) was added to the mixture and the reaction was allowed to warm to room temperature. The reaction mixture was stirred for 1 hour at room temperature. The reaction mixture was diluted with DCM and the reaction mixture was quenched with saturated aqueous NH₄Cl (100 mL). Layer were separated and the aqueous layer was extracted with EtOAc (3×50 mL), the combined organic layers were washed with brine (50 mL) and dried over MgSO₄, filtered and concentrated under reduced pressure to dryness to afford pale yellow oil. The resulting residue was purified by flash silica chromatography, elution gradient 0 to 20% EtOAc in hexanes. Product fractions were concentrated under reduced pressure to dryness to afford 1-(benzyloxy)-16-((triisopropylsilyl)oxy)hexadecan-8-one (0.200 g, 51.2%) as a colorless oil. ¹H NMR (500 MHz, CHLOROFORM-d, 27° C.) δ ppm 1.02-1.13 (21H, m), 1.24-1.45 (14H, m), 1.50-1.70 (8H, m), 2.34-2.45 (4H, t), 3.43-3.53 (2H, t), 3.62-3.76 (2H, t), 4.47-4.55 (2H, s), 7.36 (5H, m).

### Step 3: 1-(benzyloxy)-16-hydroxyhexadecan-8-one

TBAF (1.542 mL, 1.54 mmol) was added dropwise to a stirred solution of 1-(benzyloxy)-16-((triisopropylsilyl)oxy)hexadecan-8-one (0.200 g, 0.39 mmol) in THE (5 mL) at 0° C. under argon. The resulting mixture was stirred at RT for 16 hours. The reaction mixture was quenched with saturated aqueous NH₄Cl (50 mL), extracted with EtOAc (3×50 mL), the organic layer was dried over MgSO₄, filtered and concentrated under reduced pressure to dryness to afford orange oil. The resulting residue was purified by flash silica chromatography, elution gradient 0 to 40% EtOAc in hexanes. Product fractions were concentrated under reduced pressure to dryness to afford 1-(benzyloxy)-16-hydroxyhexadecan-8-one (0.124 g, 89%) as a colorless oil. ¹H NMR (500 MHz, CHLOROFORM-d, 27° C.) δ ppm 1.23-1.70 (22H, m), 2.40 (4H, t), 3.48 (2H, t), 3.66 (2H, t), 4.52 (2H, s), 7.30-7.42 (5H, m).

### Step 4: 16-(benzyloxy)-9-oxohexadecanoic acid

i) Dess-martin periodinane (435 mg, 1.03 mmol) was added in one portion to a stirred suspension of sodium bicarbonate (259 mg, 3.08 mmol) and 1-(benzyloxy)-16-hydroxyhexadecan-8-one (124 mg, 0.34 mmol) in DCM (5 mL) at 0° C. The resulting solution was allowed to come to room temp over 24 hours. The reaction mixture was diluted with DCM (20 mL), and washed sequentially with saturated aqueous NaHCO₃ (20 mL), and sat. Na₂S₂O₃ (20 mL) The organic layer was dried over MgSO₄, filtered and concentrated under reduced pressure to dryness to afford crude aldehyde precursor as a colorless dry film, which was used without further purification.

ii) The crude product was added to a stirred solution of 2-methyl-2-butene (1.087 mL, 10.26 mmol), sodium dihydrogen phosphate (246 mg, 2.05 mmol) and sodium chlorite (186 mg, 2.05 mmol) in THE (10 mL) and tert-butanol (5.00 mL) at 25° C. The resulting solution was stirred at RT for 4 hours. The reaction mixture was diluted with DCM and water (30 ml each). The reaction mixture was adjusted to pH=3 with 1 M HCl solution. The organic layer was dried over MgSO₄, filtered and concentrated under reduced pressure to dryness to afford crude product. The resulting residue was purified by flash silica chromatography, elution gradient 0 to 100% EtOAc in hexanes. Product fractions were concentrated under reduced pressure to dryness to afford 16-(benzyloxy)-9-oxohexadecanoic acid (126 mg, 98%) as a white solid. ¹H NMR (500 MHz, CHLOROFORM-d, 27° C.) δ ppm 1.24-1.45 (12H, m), 1.57 (8H, m), 2.30-2.44 (6H, m), 3.48 (2H, t), 4.52 (2H, s), 7.26-7.41 (5H, m).

### Step 5: Heptadecan-9-yl 16-(benzyloxy)-9-oxohexadecanoate

EDC (109 mg, 0.57 mmol) was added in one portion to a stirred mixture of 16-(benzyloxy)-9-oxohexadecanoic acid (125.9 mg, 0.33 mmol), heptadecan-9-ol (111 mg, 0.43 mmol), DIPEA (0.123 mL, 0.70 mmol), and DMAP (8.17 mg, 0.07 mmol) in DCM (5 mL) at 0° C. under argon. The resulting mixture was stirred at room temperature for 16 hours. The reaction mixture was diluted with 10% citric acid (25 mL) and DCM (25 mL). The layers were separated, and the aqueous layer was extracted with (DCM) (4×25 mL). The combined organic layers were dried over MgSO₄, filtered and concentrated under reduced pressure to dryness to afford crude product. The resulting residue was purified by flash silica chromatography, elution gradient 0 to 40% EtOAc in hexanes. Product fractions were concentrated under reduced pressure to dryness to afford heptadecan-9-yl 16-(benzyloxy)-9-oxohexadecanoate (126 mg, 61.3%) as a colorless oil. ¹H NMR (500 MHz, CHLOROFORM-d, 27° C.) δ ppm 0.90 (6H, t), 1.28 (48H, m), 2.26-2.32 (2H, t), 2.34-2.43 (4H, t), 3.39-3.54 (2H, t), 4.52 (2H, s), 4.89 (1H, m), 7.28-7.39 (5H, m).

### Step 6: Heptadecan-9-yl 16-hydroxy-9-oxohexadecanoate

Heptadecan-9-yl 16-(benzyloxy)-9-oxohexadecanoate (126 mg, 0.20 mmol) and Pd/C (65.4 mg, 0.06 mmol) in MeOH (5 mL) was stirred under an atmosphere of hydrogen at atmospheric pressure and RT for 16 hours. The reaction mixture was filtered through Celite. The resulting residue was purified by flash silica chromatography, elution gradient 0 to 40% EtOAc in hexanes. Product fractions were concentrated under reduced pressure to dryness to afford heptadecan-9-yl 16-hydroxy-9-oxohexadecanoate (95 mg, 88%) as a colorless oil. ¹H NMR (500 MHz, CHLOROFORM-d, 27° C.) δ ppm 0.90 (6H, t), 1.22-1.69 (48H, m), 2.25-2.33 (2H, t), 2.40 (4H, t), 3.66 (2H, t), 4.82-4.93 (1H, m).

### Step 7: Heptadecan-9-yl 16-(decanoyloxy)-9-oxohexadecanoate

EDC (38.4 mg, 0.20 mmol) was added in one portion to a stirred mixture of heptadecan-9-yl 16-hydroxy-9-oxohexadecanoate (50 mg, 0.10 mmol), decanoic acid (0.028 mL, 0.14 mmol), DIPEA (0.068 mL, 0.39 mmol), and DMAP (2.328 mg, 0.02 mmol) in DCM (5 mL) at 0° C. under argon. The resulting mixture was stirred at room temperature for 16 hours. The reaction mixture was diluted with sat. sodium bicarbonate (25 mL) and DCM (25 mL). The layers were separated, and the aqueous layer was extracted with (DCM) (4×25 mL). The combined organic layers were dried over MgSO₄, filtered, and concentrated under reduced pressure to dryness to afford crude product. The resulting residue was purified by flash silica chromatography, elution gradient 0 to 40% EtOAc in hexanes. Product fractions were concentrated under reduced pressure to dryness to afford heptadecan-9-yl 16-(decanoyloxy)-9-oxohexadecanoate (45.0 mg, 69.6%) as a colorless oil. ¹H NMR (500 MHz, CHLOROFORM-d, 27° C.) δ ppm 0.89 (9H, m), 1.20-1.42 (48H, m), 1.47-1.69 (14H, m), 2.23-2.33 (4H, t), 2.35-2.44 (4H, t), 3.98-4.13 (2H, t), 4.81-4.94 (1H, m).

### Compound 54: Heptadecan-9-yl 9-((2-oxaspiro[3.3]heptan-6-yl)amino)-16-(decanoyloxy)hexadecanoate

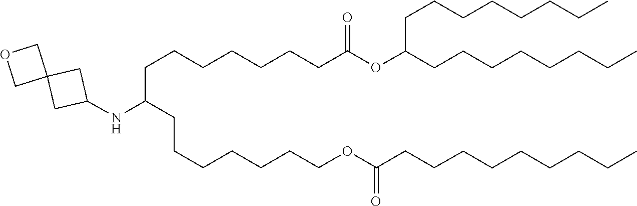 [SMILES: CCCCCCCCCC(=O)OCCCCCCCC(CCCCCCCC(=O)OC(CCCCCCCC)CCCCCCCC)NC1CC2(COC2)C1]

Sodium triacetoxyborohydride (37.9 mg, 0.18 mmol) was added in one portion to a stirred solution of heptadecan-9-yl 16-(decanoyloxy)-9-oxohexadecanoate (45 mg, 0.07 mmol) and 2-oxaspiro[3.3]heptan-6-amine hydrochloride (23.79 mg, 0.16 mmol) in DCE (2 mL) and NMP (0.5 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford heptadecan-9-yl 9-((2-oxaspiro[3.3]heptan-6-yl)amino)-16-(decanoyloxy)hexadecanoate (37.5 mg, 72.9%) as a colorless oil. ¹H NMR (500 MHz, METHANOL-d4, 27° C.) δ ppm 0.93 (9H, t), 1.32 (56H, m), 1.51-1.70 (1 OH, m), 1.96-2.03 (2H, m), 2.33 (4H, t), 2.48-2.54 (1H, m), 2.54-2.61 (2H, m), 3.16-3.27 (1H, m), 4.05-4.13 (2H, t), 4.58-4.63 (2H, s), 4.71-4.76 (2H, s), 4.93-4.98 (1H, m); C49H93NO5 m/z calcd. 776.705 observed 778.80 [M+H]+ (LCMS).

Scheme 29 below illustrates the synthetic procedures for preparing Example 55.

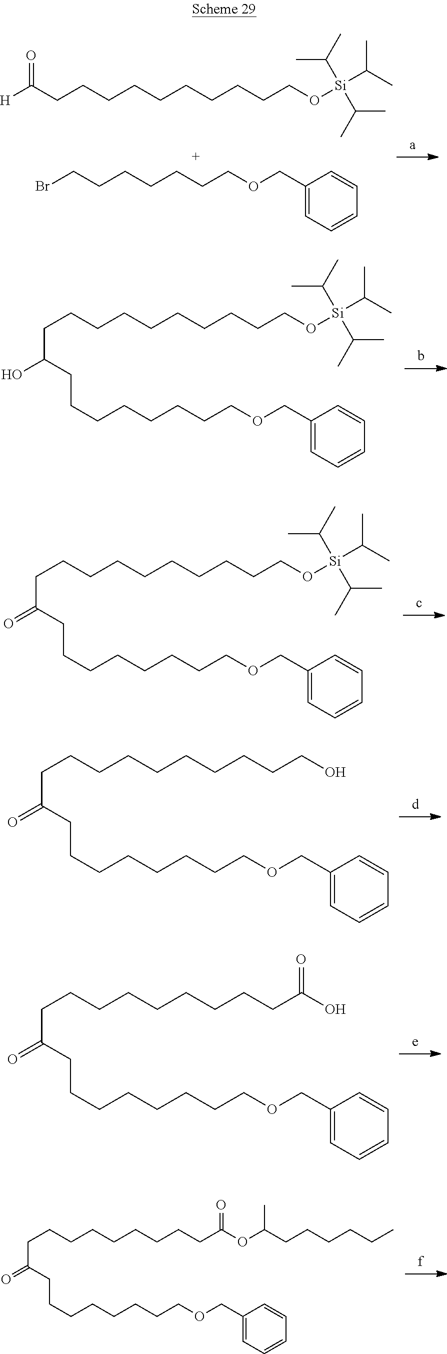

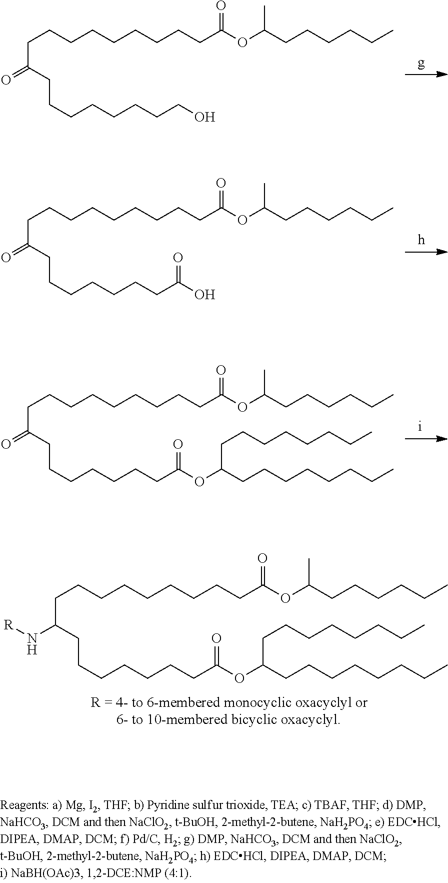

### Example 55. Synthesis of Compound 55

### Step 1: 1-(benzyloxy)-19-((triisopropylsilyl)oxy)nonadecan-9-ol

To a suspension of magnesium (0.284 g, 11.67 mmol) turnings in DMF (20 mL) containing a small iodine crystal were added few drops of the appropriate brominated compound in THE (10 mL). The mixture was heated until the reaction started, then the remaining (((8-bromooctyl)oxy)methyl)benzene (2.096 g, 7.00 mmol) was added drop by drop to maintain a non-assisted gentle reflux. After complete addition of the starting material, the mixture was heated under reflux for 1 h. The solution of Grignard reagent was cooled down and titrated prior to use. 11-((triisopropylsilyl)oxy)undecanal (2 g, 5.84 mmol) was added in one portion to the stirred mixture under argon. The resulting mixture was stirred at 70° C. for 16 hours. The reaction mixture was quenched with water (50 mL), extracted with DCM (3×25 mL), the organic layer was dried over MgSO₄, filtered and concentrated under reduced pressure to dryness to afford pale yellow oil. The resulting residue was purified by flash silica chromatography, elution gradient 0 to 40% EtOAc in hexanes. Product fractions were concentrated under reduced pressure to dryness to afford 1-(benzyloxy)-19-((triisopropylsilyl)oxy)nonadecan-9-ol (1.837 g, 74.4%) as a colorless oil. ¹H NMR (500 MHz, CHLOROFORM-d, 27° C.) δ ppm 1.03-1.14 (21H, m), 1.26-1.47 (28H, m), 1.53-1.58 (2H, m), 1.60-1.67 (2H, m), 3.44-3.54 (2H, t), 3.54-3.63 (1H, m), 3.65-3.73 (2H, t), 4.53 (2H, s), 7.36 (5H, m).

### Step 2: 1-(benzyloxy)-19-((triisopropylsilyl)oxy)nonadecan-9-one

To an oven-dried flask, 1-(benzyloxy)-19-((triisopropylsilyl)oxy)nonadecan-9-ol (2.477 g, 4.40 mmol) was dissolved in DCM (80 mL). DMSO (15 mL) was then added, followed by TEA (6.13 mL, 44.00 mmol) to the reaction mixture. The mixture was cool to RT. Pyridine sulfur trioxide was added to the mixture and the reaction was allowed to warm to room temperature. The reaction mixture was stirred for 1 hour at room temperature. The reaction mixture was diluted with DCM and the reaction mixture was quenched with saturated aqueous NH₄Cl (100 mL). Layer were separated and the aqueous layer was extracted with EtOAc (3×50 mL), the combined organic layers were washed with brine (50 mL) and dried over MgSO₄, filtered, and concentrated under reduced pressure to dryness to afford pale yellow oil. The resulting residue was purified by flash silica chromatography, elution gradient 0 to 20% EtOAc in hexanes. Product fractions were concentrated under reduced pressure to dryness to afford 1-(benzyloxy)-19-((triisopropylsilyl)oxy)nonadecan-9-one (1.837 g, 74.4%) as a colorless oil. ¹H NMR (500 MHz, CHLOROFORM-d, 27° C.) δ ppm 1.03-1.18 (21H, m), 1.29 (28H, m), 2.33-2.44 (4H, t), 3.42-3.55 (2H, t), 3.62-3.73 (2H, t), 4.52 (2H, s), 7.36 (5H, m).

### Step 3: 1-(benzyloxy)-19-hydroxynonadecan-9-one

Tetrabutylammonium fluoride (13.10 mL, 13.10 mmol) was added dropwise to a stirred solution of 1-(benzyloxy)-19-((triisopropylsilyl)oxy)nonadecan-9-one (1.837 g, 3.27 mmol) in THE (10 mL) at 0° C. under argon. The resulting mixture was stirred at RT for 16 hours. The reaction mixture was quenched with saturated aqueous NH₄Cl (50 mL), extracted with EtOAc (3×50 mL), the organic layer was dried over MgSO₄, filtered, and concentrated under reduced pressure to dryness to afford orange oil. The resulting residue was purified by flash silica chromatography, elution gradient 0 to 40% EtOAc in hexanes. Product fractions were concentrated under reduced pressure to dryness to afford 1-(benzyloxy)-19-hydroxynonadecan-9-one (1.266 g, 96%) as a colorless oil. ¹H NMR (500 MHz, CHLOROFORM-d, 27° C.) δ ppm 1.23-1.70 (30H, m), 2.40 (4H, t), 3.48 (2H, t), 3.66 (2H, t), 4.52 (2H, s), 7.30-7.42 (5H, m).

### Step 4: 19-(benzyloxy)-11-oxononadecanoic acid

i) 3-oxo-1l5-benzo[d][1,2]iodaoxole-1,1,1 (3H)-triyl triacetate (3.98 g, 9.39 mmol) was added in one portion to a stirred suspension of sodium hydrogen carbonate (2.365 g, 28.16 mmol) and 1-(benzyloxy)-19-hydroxynonadecan-9-one (1.266 g, 3.13 mmol) in DCM (20 mL) at 0° C. The resulting solution was allowed to come to room temp over 24 hours. The reaction mixture was diluted with DCM (20 mL) and washed sequentially with saturated aqueous NaHCO₃ (20 mL) and sat. Na₂S₂O₃ (20 mL) The organic layer was dried over MgSO₄, filtered, and concentrated under reduced pressure to dryness to afford crude aldehyde precursor as a colorless dry film, which was used without further purification.

ii) The crude product was added to a stirred solution of 2-methylbut-2-ene (9.94 mL, 93.86 mmol), sodium dihydrogen phosphate (2.252 g, 18.77 mmol), and sodium chlorite (1.698 g, 18.77 mmol) in THE (10 mL) and tert-butanol (5.00 mL) at 25° C. The resulting solution was stirred at RT for 4 hours. The reaction mixture was diluted with DCM and water (30 ml each). The reaction mixture was adjusted to pH=3 with 1 M HCl solution. The organic layer was dried over MgSO₄, filtered and concentrated under reduced pressure to dryness to afford crude product. The resulting residue was purified by flash silica chromatography, elution gradient 0 to 100% EtOAc in hexanes. Product fractions were concentrated under reduced pressure to dryness to afford product 19-(benzyloxy)-11-oxononadecanoic acid (1.275 g, 97%) as a white solid. ¹H NMR (500 MHz, CHLOROFORM-d, 27° C.) δ ppm 1.30 (26H, m), 2.28-2.50 (6H, m), 3.37-3.60 (2H, t), 4.53 (2H, s), 7.25-7.38 (5H, m).

### Step 5: Octan-2-yl 19-(benzyloxy)-11-oxononadecanoate

EDC (311 mg, 1.62 mmol) was added in one portion to a stirred mixture of 19-(benzyloxy)-11-oxononadecanoic acid (400 mg, 0.96 mmol), octan-2-ol (0.195 mL, 1.24 mmol), DIPEA (0.350 mL, 2.01 mmol), and DMAP (23.35 mg, 0.19 mmol) in DCM (5 mL) at 0° C. under argon. The resulting mixture was stirred at room temperature for 16 hours. The reaction mixture was diluted with sat. sodium bicarbonate (25 mL) and DCM (25 mL). The layers were separated, and the aqueous layer was extracted with (DCM) (4×25 mL). The combined organic layers were dried over MgSO₄, filtered, and concentrated under reduced pressure to dryness to afford crude product. The resulting residue was purified by flash silica chromatography, elution gradient 0 to 40% EtOAc in hexanes. Product fractions were concentrated under reduced pressure to dryness to afford octan-2-yl 19-(benzyloxy)-11-oxononadecanoate (410 mg, 81%) as a colorless oil. ¹H NMR (500 MHz, CHLOROFORM-d, 27° C.) δ ppm 0.83-0.97 (3H, m), 1.15-1.67 (39H, m), 2.18-2.31 (2H, m), 2.39 (4H, t), 3.39-3.54 (2H, m), 4.52 (2H, s), 4.80-5.00 (1H, m), 7.28-7.40 (5H, m).

### Step 6: Octan-2-yl 19-hydroxy-11-oxononadecanoate

Octan-2-yl 19-(benzyloxy)-11-oxononadecanoate (410 mg, 0.77 mmol) and Pd/C (247 mg, 0.23 mmol) in MeOH (10 mL) was stirred under an atmosphere of hydrogen and RT for 16 hours. The resulting residue was purified by flash silica chromatography, elution gradient 0 to 60% EtOAc in hexanes. Product fractions were concentrated under reduced pressure to dryness to afford octan-2-yl 19-hydroxy-11-oxononadecanoate (200 mg, 58.8%) as a pale yellow oil. ¹H NMR (500 MHz, CHLOROFORM-d, 27° C.) δ ppm 0.92 (3H, t), 1.17-1.68 (39H, m), 2.22-2.32 (2H, m), 2.40 (4H, t), 3.55-3.67 (2H, m), 4.90 (1H, m).

### Step 7: 19-(octan-2-yloxy)-9,19-dioxononadecanoic acid

i) DMP (577 mg, 1.36 mmol) was added in one portion to a stirred suspension of sodium bicarbonate (343 mg, 4.08 mmol) and octan-2-yl 19-hydroxy-11-oxononadecanoate (200 mg, 0.45 mmol) in DCM (5 mL) at 0° C. The resulting solution was allowed to come to room temp over 24 hours. The reaction mixture was diluted with DCM (20 mL), and washed sequentially with saturated aqueous NaHCO₃ (20 mL), and sat. Na₂S₂O₃ (20 mL) The organic layer was dried over MgSO₄, filtered, and concentrated under reduced pressure to dryness to afford crude aldehyde precursor as a colorless dry film, which was used without further purification.

ii) The crude product was added to a stirred solution of 2-methylbut-2-ene (1.442 mL, 13.61 mmol), sodium dihydrogen phosphate (327 mg, 2.72 mmol), and sodium chlorite (246 mg, 2.72 mmol) in THE (10 mL) and tert-butanol (5.00 mL) at 25° C. The resulting solution was stirred at RT for 4 hours. The reaction mixture was diluted with DCM and water (30 ml each). The reaction mixture was adjusted to pH=3 with 1 M HCl solution. The organic layer was dried over MgSO₄, filtered, and concentrated under reduced pressure to dryness to afford crude product. The resulting residue was purified by flash silica chromatography, elution gradient 0 to 100% EtOAc in hexanes. Product fractions were concentrated under reduced pressure to dryness to afford desired product 19-(octan-2-yloxy)-9,19-dioxononadecanoic acid as a white solid. ¹H NMR (500 MHz, CHLOROFORM-d, 27° C.) δ ppm 0.92 (3H, t), 1.24-1.72 (38H, m), 2.29 (2H, t), 2.35-2.47 (6H, m), 4.87 (1H, m).

### Step 8: 1-(heptadecan-9-yl) 19-(octan-2-yl) 9-oxononadecanedioate

EDC (227 mg, 1.18 mmol) was added in one portion to a stirred mixture of 19-(octan-2-yloxy)-9,19-dioxononadecanoic acid (256 mg, 0.56 mmol), heptadecan-9-ol (217 mg, 0.84 mmol), DIPEA (0.403 mL, 2.31 mmol), and DMAP (13.76 mg, 0.11 mmol) in DCM (5 mL) at 0° C. under argon. The resulting mixture was stirred at room temperature for 16 hours. The reaction mixture was diluted with sat. sodium bicarbonate (25 mL) and DCM (25 mL). The layers were separated, and the aqueous layer was extracted with (DCM) (4×25 mL). The combined organic layers were dried over MgSO₄, filtered, and concentrated under reduced pressure to dryness to afford crude product. The resulting residue was purified by flash silica chromatography, elution gradient 0 to 40% EtOAc in hexanes. Product fractions were concentrated under reduced pressure to dryness to afford 1-(heptadecan-9-yl) 19-(octan-2-yl) 9-oxononadecanedioate (141 mg, 36.1%) as a colorless oil. ¹H NMR (500 MHz, CHLOROFORM-d, 27° C.) δ ppm 0.86-0.95 (9H, m), 1.18-1.69 (65H, m), 2.29 (4H, m), 2.35-2.44 (4H, m), 4.80-4.98 (2H, m).

### Compound 55: 1-(heptadecan-9-yl) 19-(octan-2-yl) 9-((2-oxaspiro[3.3]heptan-6-yl)amino)nonadecanedioate

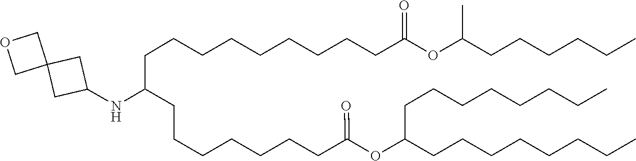 [SMILES: CCCCCCCCC(CCCCCCCC)OC(=O)CCCCCCCC(CCCCCCCCCC(=O)OC(C)CCCCCC)NC1CC2(COC2)C1]

Sodium triacetoxyborohydride (0.116 g, 0.55 mmol) was added in one portion to a stirred solution of 1-(heptadecan-9-yl) 19-(octan-2-yl) 9-oxononadecanedioate (0.141 g, 0.20 mmol) and 2-oxaspiro[3.3]heptan-6-amine hydrochloride (0.073 g, 0.49 mmol) in DCE (2 mL) and NMP (0.5 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered, and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford 1-(heptadecan-9-yl) 19-(octan-2-yl) 9-((2-oxaspiro[3.3]heptan-6-yl)amino)nonadecanedioate (0.099 g, 61.7%) as a colorless oil. ¹H NMR (500 MHz, METHANOL-d4, 27° C.) δ ppm 0.92 (9H, t), 1.18-1.71 (71H, m), 1.91-2.04 (2H, m), 2.27-2.37 (4H, m), 2.43-2.50 (1H, m), 2.52-2.59 (2H, m), 3.11-3.23 (1H, m), 4.56-4.64 (2H, s), 4.73 (2H, s); C49H93NO5 m/z calcd. 789.721 observed 790.80 [M+H]+ (LCMS).

Scheme 30 below illustrates the synthetic procedures for preparing Example 56.

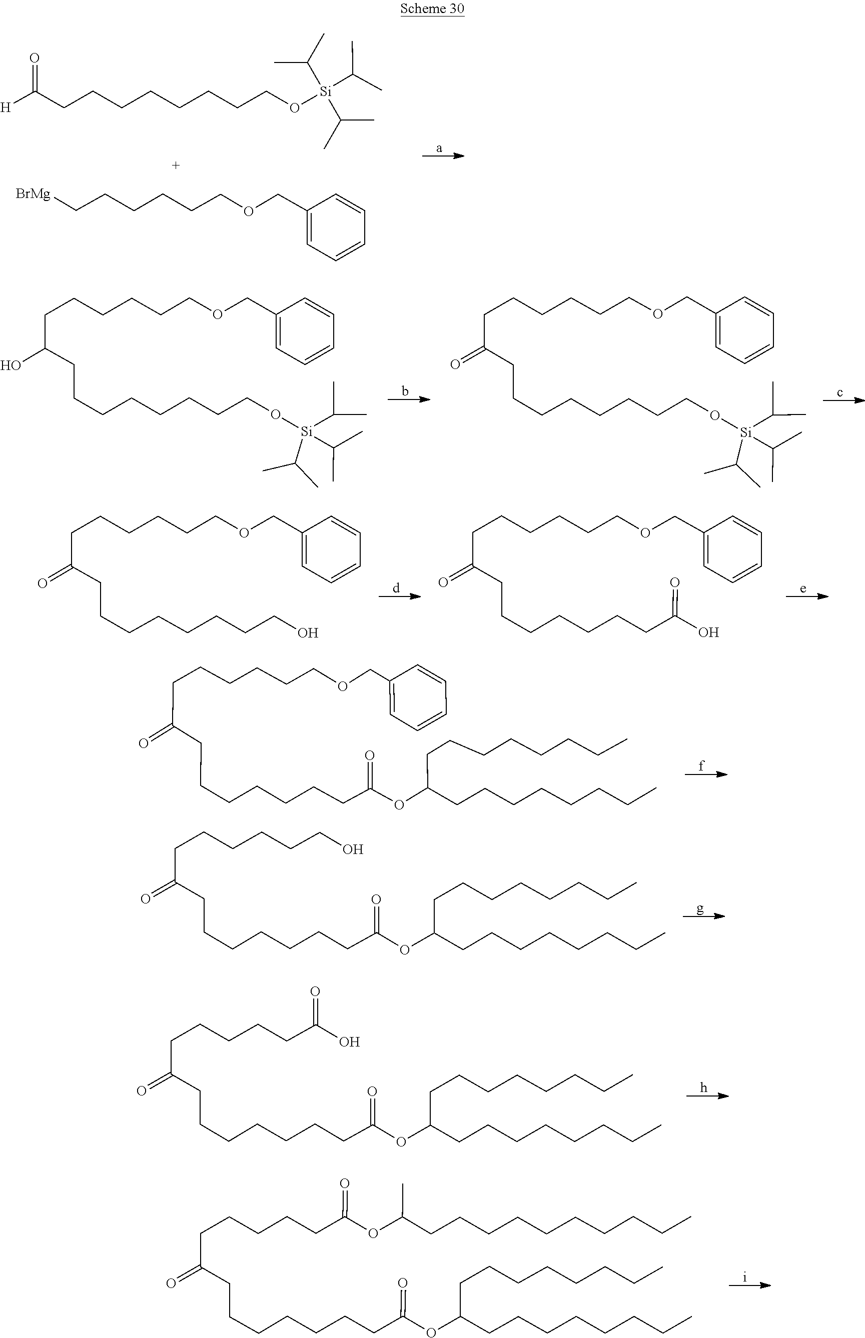

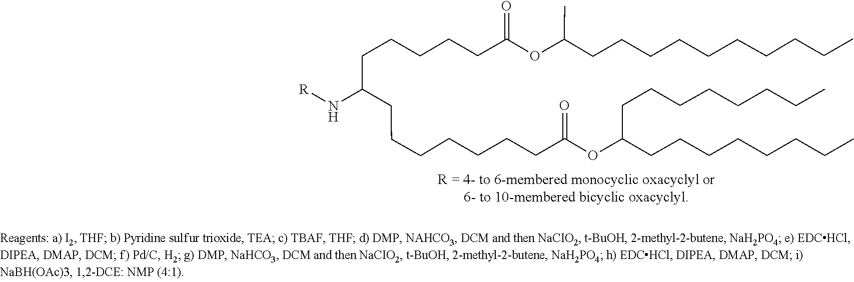

### Example 56. Synthesis of Compound 56

### Step 1: 1-(benzyloxy)-19-((triisopropylsilyl)oxy)nonadecan-9-ol

(6-(benzyloxy)hexyl)magnesium bromide (5.79 mL, 2.89 mmol) was diluted in THF (10 mL) containing a small crystal of iodine. The solution of Grignard reagent was cooled down to 0 C. 9-((triisopropylsilyl)oxy)nonanal (0.7 g, 2.23 mmol) was added in one portion to the stirred mixture under argon at 0 C. The resulting mixture was stirred at 70° C. for 16 hours. The reaction mixture was quenched with water (50 mL), extracted with DCM (3×25 mL), the organic layer was dried over MgSO₄, filtered, and concentrated under reduced pressure to dryness to afford pale yellow oil. The resulting residue was purified by flash silica chromatography, elution gradient 0 to 40% EtOAc in hexanes. Product fractions were concentrated under reduced pressure to dryness to afford 1-(benzyloxy)-15-((triisopropylsilyl)oxy)pentadecan-7-ol (0.550 g, 49%) as a colorless oil. ¹H NMR (500 MHz, CHLOROFORM-d, 27° C.) δ ppm 1.04-1.13 (21H, m), 1.25-1.49 (20H, m), 1.51-1.60 (2H, m), 1.60-1.69 (2H, m), 3.43-3.53 (2H, t), 3.55-3.63 (1H, m), 3.66-3.73 (2H, t), 4.53 (2H, s), 7.36 (5H, m).

### Step 2: 1-(benzyloxy)-19-((triisopropylsilyl)oxy)nonadecan-9-one

To an oven-dried flask, 1-(benzyloxy)-15-((triisopropylsilyl)oxy)pentadecan-7-ol (0.860 g, 1.70 mmol) was dissolved in DCM (40 mL). DMSO (7.50 mL) was then added, followed by TEA (2.365 mL, 16.97 mmol) to the reaction mixture. The mixture was cool to 0 C. pyridine sulfur trioxide (2.160 g, 13.57 mmol) was added to the mixture and the reaction was allowed to warm to room temperature. The reaction mixture was stirred for 1 hour at room temperature. The reaction mixture was diluted with DCM and the reaction mixture was quenched with saturated aqueous NH₄Cl (100 mL). Layer were separated and the aqueous layer was extracted with EtOAc (3×50 mL), the combined organic layers were washed with brine (50 mL) and dried over MgSO₄, filtered and concentrated under reduced pressure to dryness to afford pale yellow oil. The resulting residue was purified by flash silica chromatography, elution gradient 0 to 20% EtOAc in hexanes. Product fractions were concentrated under reduced pressure to dryness to afford 1-(benzyloxy)-15-((triisopropylsilyl)oxy)pentadecan-7-one (0.684 g, 80%) as a colorless oil. ¹H NMR (500 MHz, CHLOROFORM-d, 27° C.) δ ppm 0.98-1.15 (21H, m), 1.25-1.71 (20H, m), 2.32-2.50 (4H, m), 3.42-3.52 (2H, t), 3.62-3.74 (2H, t), 4.52 (2H, s), 7.36 (5H, m).

### Step 3: 1-(benzyloxy)-15-hydroxypentadecan-7-one

Tetrabutylammonium fluoride (5.42 mL, 5.42 mmol) was added dropwise to a stirred solution of 1-(benzyloxy)-15-((triisopropylsilyl)oxy)pentadecan-7-one (0.684 g, 1.35 mmol) in THE (10 mL) at 0° C. under argon. The resulting mixture was stirred at RT for 16 hours. The reaction mixture was quenched with saturated aqueous NH₄Cl (50 mL), extracted with EtOAc (3×50 mL), the organic layer was dried over MgSO₄, filtered and concentrated under reduced pressure to dryness to afford orange oil. The resulting residue was purified by flash silica chromatography, elution gradient 0 to 40% EtOAc in hexanes. Product fractions were concentrated under reduced pressure to dryness to afford 1-(benzyloxy)-15-hydroxypentadecan-7-one (0.385 g, 82%) as a colorless oil. ¹H NMR (500 MHz, CHLOROFORM-d, 27° C.) δ ppm 1.23-1.70 (20H, m), 2.40 (4H, t), 3.48 (2H, t), 3.66 (2H, t), 4.52 (2H, s), 7.30-7.42 (5H, m).

### Step 4: 15-(benzyloxy)-9-oxopentadecanoic acid

i) 3-oxo-1l5-benzo[d][1,2]iodaoxole-1,1,1 (3H)-triyl triacetate (1.515 g, 3.57 mmol) was added in one portion to a stirred suspension of sodium bicarbonate (0.900 g, 10.72 mmol) and 1-(benzyloxy)-15-hydroxypentadecan-7-one (0.415 g, 1.19 mmol) in DCM (10 mL) at 0° C. The resulting solution was allowed to come to room temp over 24 hours. The reaction mixture was diluted with DCM (20 mL), and washed sequentially with saturated aqueous NaHCO₃ (20 mL), and sat. Na₂S₂O₃ (20 mL) The organic layer was dried over MgSO₄, filtered, and concentrated under reduced pressure to dryness to afford crude aldehyde precursor as a colorless dry film, which was used without further purification.

ii) The crude product was added to a stirred solution of 2-methylbut-2-ene (3.78 mL, 35.72 mmol), sodium dihydrogen phosphate (0.857 g, 7.14 mmol) and sodium chlorite (0.646 g, 7.14 mmol) in THE (10.00 mL) and tBuOH (5 mL) at 25° C. The resulting solution was stirred at RT for 4 hours. The reaction mixture was diluted with DCM and water (30 ml each). The reaction mixture was adjusted to pH=3 with 1 M HCl solution. The organic layer was dried over MgSO₄, filtered and concentrated under reduced pressure to dryness to afford crude product. The resulting residue was purified by flash silica chromatography, elution gradient 0 to 100% EtOAc in hexanes. Product fractions were concentrated under reduced pressure to dryness to afford desired product 15-(benzyloxy)-9-oxopentadecanoic acid (0.433 g, 100%) as a white solid. ¹H NMR (500 MHz, CHLOROFORM-d, 27° C.) δ ppm 1.29 (18H, m), 2.39 (6H, m), 3.48 (2H t), 4.52 (2H, s), 7.30-7.42 (5H, m).

### Step 5: Heptadecan-9-yl 15-(benzyloxy)-9-oxopentadecanoate

EDC (389 mg, 2.03 mmol) was added in one portion to a stirred mixture of 15-(benzyloxy)-9-oxopentadecanoic acid (433 mg, 1.19 mmol), heptadecan-9-ol (398 mg, 1.55 mmol), DIPEA (0.438 mL, 2.51 mmol), and DMAP (29.2 mg, 0.24 mmol) in DCM (5 mL) at 0° C. under argon. The resulting mixture was stirred at room temperature for 16 hours. The reaction mixture was diluted with sat. sodium bicarbonate (25 mL) and DCM (25 mL). The layers were separated, and the aqueous layer was extracted with (DCM) (4×25 mL). The combined organic layers were dried over MgSO₄, filtered and concentrated under reduced pressure to dryness to afford crude product. The resulting residue was purified by flash silica chromatography, elution gradient 0 to 40% EtOAc in hexanes. Product fractions were concentrated under reduced pressure to dryness to afford heptadecan-9-yl 15-(benzyloxy)-9-oxopentadecanoate (276 mg, 38.4%) as a colorless oil. ¹H NMR (500 MHz, CHLOROFORM-d, 27° C.) δ ppm 0.83-0.95 (6H, t), 1.21-1.70 (46H, m), 2.29 (2H, t), 2.36-2.45 (4H, m), 3.42-3.53 (2H, t), 4.52 (2H, s), 4.89 (1H, m), 7.35 (5H, m).

### Step 6: Heptadecan-9-yl 15-hydroxy-9-oxopentadecanoate

Heptadecan-9-yl 15-(benzyloxy)-9-oxopentadecanoate (276 mg, 0.46 mmol) and Pd/C (147 mg, 0.14 mmol) in MeOH (10 mL) was stirred under an atmosphere of hydrogen and RT for 16 hours. The resulting residue was purified by flash silica chromatography, elution gradient 0 to 60% EtOAc in hexanes. Product fractions were concentrated under reduced pressure to dryness to afford heptadecan-9-yl 15-hydroxy-9-oxopentadecanoate as a pale yellow oil. ¹H NMR (500 MHz, CHLOROFORM-d, 27° C.) δ ppm 0.92 (6H, t), 1.23-1.71 (46H, m), 2.24-2.32 (2H, t), 2.37-2.45 (4H, m), 3.54-3.70 (2H, m), 4.87 (1H, m).

### Step 7: 15-(benzyloxy)-9-oxopentadecanoic acid

i) 3-oxo-1l5-benzo[d][1,2]iodaoxole-1,1,1 (3H)-triyl triacetate (473 mg, 1.12 mmol) was added in one portion to a stirred suspension of sodium bicarbonate (281 mg, 3.35 mmol) and heptadecan-9-yl 15-hydroxy-9-oxopentadecanoate (190 mg, 0.37 mmol) in DCM (10 mL) at 0° C. The resulting solution was allowed to come to room temp over 24 hours. The reaction mixture was diluted with DCM (20 mL), and washed sequentially with saturated aqueous NaHCO₃ (20 mL), and sat. Na₂S₂O₃ (20 mL) The organic layer was dried over MgSO₄, filtered and concentrated under reduced pressure to dryness to afford crude aldehyde precursor as a colorless dry film, which was used without further purification.

ii) The crude product was added to a stirred solution of 2-methylbut-2-ene (1.182 mL, 11.16 mmol), sodium dihydrogen phosphate (268 mg, 2.23 mmol), and sodium bicarbonate (281 mg, 3.35 mmol) in THE (10.00 mL) and tBuOH (5 mL) at 25° C. The resulting solution was stirred at RT for 4 hours. The reaction mixture was diluted with DCM and water (30 ml each). The reaction mixture was adjusted to pH=3 with 1 M HCl solution. The organic layer was dried over MgSO₄, filtered, and concentrated under reduced pressure to dryness to afford crude product. The resulting residue was purified by flash silica chromatography, elution gradient 0 to 100% EtOAc in hexanes. Product fractions were concentrated under reduced pressure to dryness to afford 15-(benzyloxy)-9-oxopentadecanoic acid (0.433 g, 100%) as a white solid. ¹H NMR (500 MHz, CHLOROFORM-d, 27° C.) δ ppm 0.92 (6H, t), 1.24-1.72 (44H, m), 2.29 (2H, t), 2.35-2.47 (6H, m), 4.87 (1H, m).

### Step 8: 1-(dodecan-2-yl) 15-(heptadecan-9-yl) 7-oxopentadecanedioate

EDC (153 mg, 0.80 mmol) was added in one portion to a stirred mixture of 15-(heptadecan-9-yloxy)-7,15-dioxopentadecanoic acid (200 mg, 0.38 mmol), dodecan-2-ol (0.128 mL, 0.57 mmol), DIPEA (0.273 mL, 1.56 mmol), and DMAP (9.31 mg, 0.08 mmol) in DCM (5 mL) at 0° C. under argon. The resulting mixture was stirred at room temperature for 16 hours. The reaction mixture was diluted with sat. sodium bicarbonate (25 mL) and DCM (25 mL). The layers were separated, and the aqueous layer was extracted with (DCM) (4×25 mL). The combined organic layers were dried over MgSO₄, filtered, and concentrated under reduced pressure to dryness to afford crude product. The resulting residue was purified by flash silica chromatography, elution gradient 0 to 40% EtOAc in hexanes. Product fractions were concentrated under reduced pressure to dryness to afford 1-(dodecan-2-yl) 15-(heptadecan-9-yl) 7-oxopentadecanedioate as a colorless oil. ¹H NMR (500 MHz, CHLOROFORM-d, 27° C.) δ ppm 0.90 (9H, t), 1.17-1.72 (66H, m), 2.29 (4H, t), 2.40 (4H, m), 4.90 (1H, m).

### Compound 56: 1-(dodecan-2-yl) 15-(heptadecan-9-yl) 7-((2-oxaspiro[3.3]heptan-6-yl)amino)pentadecanedioate

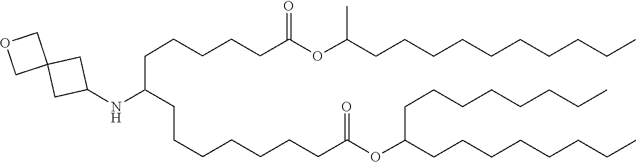 [SMILES: CCCCCCCCCCC(C)OC(=O)CCCCCC(CCCCCCCC(=O)OC(CCCCCCCC)CCCCCCCC)NC1CC2(COC2)C1]

Sodium triacetoxyborohydride (103 mg, 0.49 mmol) was added in one portion to a stirred solution of 1-(dodecan-2-yl) 15-(heptadecan-9-yl) 7-oxopentadecanedioate (125 mg, 0.18 mmol) and 2-oxaspiro[3.3]heptan-6-amine hydrochloride (64.8 mg, 0.43 mmol) in DCE (2 mL) and NMP (0.5 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford 1-(dodecan-2-yl) 15-(heptadecan-9-yl) 7-((2-oxaspiro[3.3]heptan-6-yl)amino)pentadecanedioate (85 mg, 59.9%) as a colorless oil. ¹H NMR (500 MHz, METHANOL-d4, 27° C.) δ ppm 0.92 (9H, t), 1.31 (71H, m), 1.91-2.05 (2H, m), 2.26-2.37 (4H, m), 2.43-2.50 (1H, m), 2.52-2.60 (2H, m), 3.11-3.23 (1H, m), 4.53-4.63 (2H, s), 4.69-4.78 (2H, s); C49H93NO5 m/z calcd. 789.721 observed 790.80 [M+H]+ (LCMS).

Scheme 31 below demonstrates the synthetic procedures for preparing Example 57.

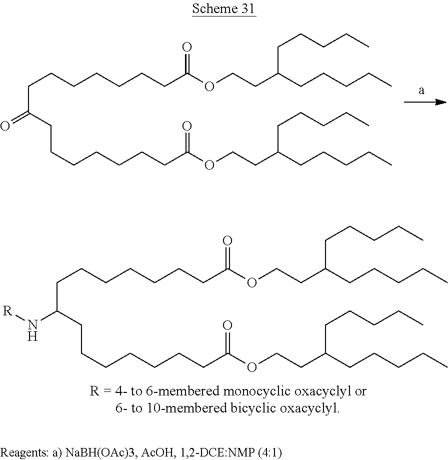

### Example 57. Synthesis of Compound 57

### Compound 57: Bis(3-pentyloctyl) 9-((6-oxaspiro[3.4]octan-2-yl)amino)heptadecanedioate

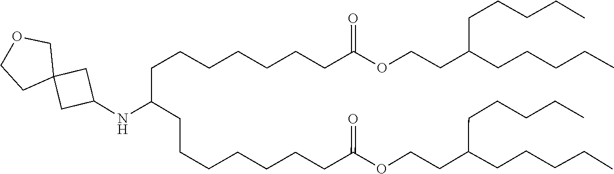 [SMILES: CCCCCC(CCCCC)CCOC(=O)CCCCCCCC(CCCCCCCC(=O)OCCC(CCCCC)CCCCC)NC1CC2(CCOC2)C1]

Bis(3-pentyloctyl) 9-oxoheptadecanedioate was prepared following the protocol described for Example 1. Then, sodium triacetoxyhydroborate (76 mg, 0.36 mmol) was added in one portion to a stirred solution of bis(3-pentyloctyl) 9-oxoheptadecanedioate (90 mg, 0.13 mmol) and 6-oxaspiro[3.4]octan-2-amine (40.5 mg, 0.32 mmol) in DCE (2 mL) and NMP (0.5 mL) at 0° C. under argon. The resulting solution was stirred at room temperature for 16 hours. The reaction mixture was diluted with DCM (50 mL) and sat. sodium carbonate (50 mL). The layers were separated, and the aqueous layer was extracted with (DCM) (3×25 mL). The organic layer was dried over MgSO₄, filtered, and concentrated under reduced pressure to dryness to afford crude product. The resulting residue was purified by flash silica chromatography, elution gradient 0 to 100% (20% MeOH and 1% NH₄OH in DCM) in DCM. Product fractions were concentrated under reduced pressure to dryness to afford bis(3-pentyloctyl) 9-((6-oxaspiro[3.4]octan-2-yl)amino)heptadecanedioate (87 mg, 83%) as a colorless oil. ¹H NMR (500 MHz, METHANOL-d4, 27° C.) δ ppm 0.93 (12H, t), 1.25-1.69 (62H, m), 1.83-2.04 (4H, m), 2.23-2.36 (6H, m), 2.47-2.57 (1H, m), 3.33-3.44 (1H, m), 3.58-3.73 (2H, m), 3.73-3.84 (2H, m), 4.12 (4H, t); C50H95NO5 m/z calcd. 789.721 observed 790.9 [M+H]+ (LCMS).

### Comparative Example 1. Synthesis of [(10Z,13Z)-1-[(9Z,12Z)-octadeca-9,12-dienyl]nonadeca-10,13-dienyl] 4-(dimethylamino)butanoate (DLin-MC3-DMA or MC3)

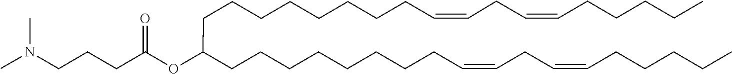 [SMILES: CCCCC/C=C\C/C=C\CCCCCCCCC(CCCCCCCC/C=C\C/C=C\CCCCC)OC(=O)CCCN(C)C]

DLin-MC3-DMA (MC3) was prepared following the method described in WO2010144740 (Example 5, p140). ¹H NMR (400 MHz, CDCl₃) δ 5.27-5.45 (m, 8H), 4.81-4.93 (m, 1H), 2.78 (t, 4H), 2.32 (q, 4H), 2.24 (s, 6H), 2.05 (q, 8H), 1.81 (q, 2H), 1.44-1.59 (m, 4H), 1.21-1.45 (m, 36H), 0.90 (t, 6H). Expected Number of Hs: 79; assigned Hs: 79. LCMS m/z 642.5 [M+H]⁺.

### Comparative Example 2. Synthesis of heptadecan-9-yl 8-((2-hydroxyethyl)(8-(nonyloxy)-8-oxooctyl)amino)octanoate (MOD5)

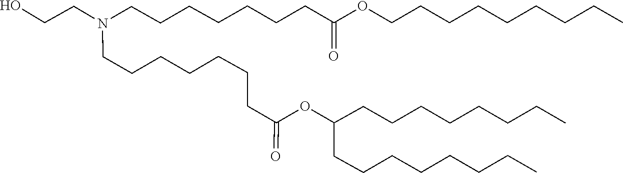 [SMILES: CCCCCCCCCOC(=O)CCCCCCCN(CCO)CCCCCCCC(=O)OC(CCCCCCCC)CCCCCCCC]

MOD5 was prepared according to the procedure for Lipid 5 in Sabnis et al. (Mol Ther. 2018, 26(6), pages 1509-1519).

### Comparative Example 3. Synthesis of heptadecan-9-yl 8-((2-hydroxyethyl)(6-oxo-6-(undecyloxy)hexyl)amino)octanoate (MOD8)

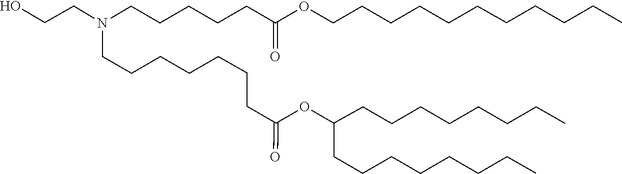 [SMILES: CCCCCCCCCCCOC(=O)CCCCCN(CCO)CCCCCCCC(=O)OC(CCCCCCCC)CCCCCCCC]

MOD8 was prepared according to the procedure for Lipid 8 in Sabnis et al. (Mol Ther. 2018, 26(6), pages 1509-1519).

### Example 58. Preparation of Lipid Nanoparticle (LNP) Formulations

A solution of eGFP mRNA (purchased from TriLink Biotechnologies) in citrate buffer was prepared by mixing mRNA dissolved in MilliQ-water, 100 mM citrate buffer (pH 3) and MilliQ-water to give a solution of 50 mM citrate. Lipid solution in ethanol (99.5%) was prepared with four different lipid components: ionizable lipid (see Table 1); cholesterol (Sigma-Aldrich); DSPC (distearoyl phosphatidyl choline, Avanti Polar Lipids Inc); and a polymer-conjugated lipid (see Table 1). The ratio of lipids in all experiments was ionizable lipid/cholesterol/DSPC/polymer-conjugated lipid (50/38.5/10/1.5 mol %). The total concentration of lipids in all experiments was 12.5 mM.

The mRNA and lipid solutions were mixed in a NanoAssemblr (Precision Nanosystems, Vancouver, BC, Canada) microfluidic mixing system at a mixing ratio of Aq:EtOH=3:1 and a constant flow rate of 12 mL/min. The mRNA in citrate buffer solution was prepared such that, at the time of mixing the ratio between the nitrogen atoms on the ionizable lipid and phosphorus atoms (N/P ratio) on the mRNA chain was either 3:1 or 6:1 (see Table 1).

The first 0.2-0.35 mL and the last 0.05-0.1 mL of the LNP suspension prepared were discarded while the rest of the volume was collected as the sample fraction. The size of the mRNA lipid nanoparticles was determined by dynamic light scattering measurements using a Zetasizer Nano ZS from Malvern Instruments Ltd, giving directly the z-average particle diameter. The number-weighted particle size distributions and averages were calculated using a particle refractive index of 1.45.

The final mRNA concentration and encapsulation efficiency percentage (% EE) was measured by Quant-it Ribogreen Assay Kit (ThermoFischer Scientific Inc.) using Triton-X100 to disrupt the LNPs. The mRNA encapsulation efficiency was determined according to the following equation:

$$\% E E = [ 1 - ( \frac{non - encapsulated mRNA}{encaspulated mRNA} ) ] \times 100$$

Table 1 summarizes the characterization of LNP formulations comprising Compound 1 or MC3.

TABLE 1
Characterization of LNP compositions

| For- | | | | | | |
|---|---|---|---|---|---|---|
| mulation | Ionizable | N/P | Polymer- | % | <d>_{Z} | <d>_{N} |
| No. | lipid | ratio | conjugated lipid | EE | (nm) | (nm) |
| 1 | Compound 1 | 3:1 | DMPE-PEG2000 | 97 | 84 | 68 |
| 2 | Compound 1 | 6:1 | DMPE-PEG2000 | 94 | 74 | 57 |
| 3 | Compound 2 | 3:1 | DMPE-PEG2000 | 96 | 73 | 58 |
| 4 | Compound 3 | 3:1 | DMPE-PEG2000 | 99 | 79 | 58 |
| 5 | Compound 4 | 3:1 | DMPE-PEG2000 | 96 | 81 | 62 |
| 6 | Compound 5 | 3:1 | DMPE-PEG2000 | 99 | 70 | 54 |
| 7 | MC3 | 3:1 | DMPE-PEG2000 | 99 | 86 | 72 |
| 8 | MOD5 | 6:1 | DMG-PEG2000 | 98 | 66 | 47 |
| 9 | MOD8 | 6:1 | DMG-PEG2000 | 98 | 73 | 49 |

DMPE-PEG2000 is dimyristoyl phosphatidyl ethanolamine-poly(ethylene glycol) 2000 (obtained from NOF Corporation).
DMG-PEG2000 is 1,2-dimyristoyl-rac-glycero-3-methoxypolyethylene glycol-2000.

### Example 59. In Vitro Expression of eGFP in 16HBE Cells

In vitro expression of EGFP protein from LNP formulation nos. 1 and 7 described in Example 58 was tested in the human broncho-epithelial cell line 16HBE (Sigma-Aldrich SCC150. 16HBE cells were maintained in DMEM, low glucose with GlutaMAX™+pyruvate (Gibco 21885-025) supplemented with MEM Non-Essential Amino Acids (Gibco 11140035) and 10% Heat-Inactivated Fetal Calf Serum (HI-FCS). The cells were cultured at 37° C. in a humidified atmosphere with 5% CO2. The day prior to an experiment, cells were detached from culture flasks using TrypLE™ (Gibco 12604013) and seeded in cell culture treated 96-well plates (Greiner Bio-One #655090) at a density of 20.000 cells per well. At the day of an experiment, 1 hour before incubation with LNPs, medium was removed and replaced with 95 ul DMEM including 1% HI-FCS. After 1 hour of conditioning, 5 ul of LNPs in PBS was added and mixed by a Bravo robot, resulting in a final concentration of 50-125 ng mRNA per well. Cells were then incubated for 24 h at 37° C. in a humidified atmosphere with 5% CO2. Absolute quantification of EGFP protein was done using ELISA (GFP SimpleStep ELISA kit, Abcam #ab171581). After 24 h of incubation, cells were lysed by adding 100 ul of lysis buffer to each well (Lysis buffer included in the kit, or Abcam products #ab193970 and #ab193971) Cells were then lysed by a freeze-thaw cycle in lysis buffer. Lysates were measured in appropriate dilutions according to the kit manufacturer's instructions. Results are reported as number of EGFP molecules expressed per mRNA dosed, after 24 h, calculated from the values of 125 ng mRNA per well. Results are plotted as Mean+SEM from triplicate values. The results are summarized in Table 2.

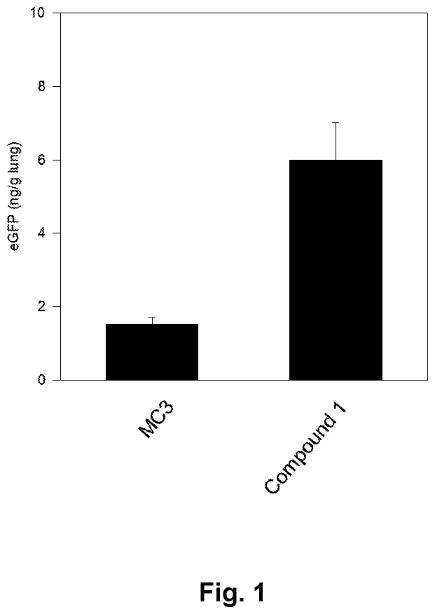

The expression of eGFP in 16HBE cytosol proves the function of the used LNP composition of eGFP mRNA in promoting gene transfection and transcription. The quantification of eGFP allows to compare different LNP compositions with the MC3-based lipid composition, used as a comparator, with regard to the induction of protein expression in 16HBE to obtain the ratio of lipid to MC3. In this application, the LNP compositions differ in their ionizable lipid (IL) component. Thus, the expression results indicate the potential of the ionizable lipid described here for applications in gene therapy. (see FIG. 1).

TABLE 2
eGFP expression 16HBE

| | | | Ratio |
|---|---|---|---|
| Formulation | | eGFP in | Lipid/ |
| no. | Lipid | 16HBE* | MC3 |
| 1 | Compound 1 | 0.044 (±0.004) | 1.9 |
| 7 | MC3 | 0.023 (±0.001) | 1 |

*number of protein molecules per number of dosed mRNA molecules (standard error of the mean).

### Example 60. In Vivo Intratracheal Administration of LNP Formulations to Rat

All experiments were performed in accordance with Swedish Animal Welfare and were approved by the Ethical Committee for Laboratory Animals in Gothenburg, Sweden. Male Wistar rats were purchased from Charles River Laboratories (Germany) at an average body weight of 250 g were. For the intratracheal (i.t.) treatment with either PBS or LNP formulations (Formulations 1 and 7 of Example 58) the rats were anaesthetised with an isoflurane mixture (air/oxygen and 4% isoflurane), put in a supine position with 30-40° angle and instilled with using a modified metal cannula with a bolus-bulb on the top. Following the i.t. dosing, rats were placed in cages in a supine position with their head up until regained consciousness. The instillation volume was 1 ml/kg rat. 24 hours after treatment the rats were terminated by an i.p injection of Allfatal vet (100 mg/ml) and cutting of the vena cava.

### Determination of Cytokine Levels in Rat BALF

Broncheo-alveolar lavage (BAL) was performed by manual perfusion of the whole lung. After the trachea had been exposed a polyethylene tube (PE120) was inserted and ligated with a 1-0 silk suture. The tube was connected to a syringe, prefilled with 4 ml of PBS at room temperature, and PBS was slowly injected into the lung. The BAL (BALF) fluid was recollected by slow aspiration into the syringe, then slowly re-injected into the lung and finally withdrawn and transferred to a test tube.

Tubes with BALF samples were kept on ice until centrifugation (Hettich ROTANTA 46R, 1200 rpm, 10 min, 4° C.). Following centrifugation the supernatant was removed and the cell pellet was resuspended in 0.5 ml of PBS, kept on ice and immediately processed to the cell counting. The total and differential number of cells was counted using an automated Hematology Analyzer SYSMEX XT-1800i Vet (Sysmex, Kobe Japan). Prior to Sysmex analysis the cell suspension was vortexed.

Determination of eGFP Protein in Lung Tissue Homogenates

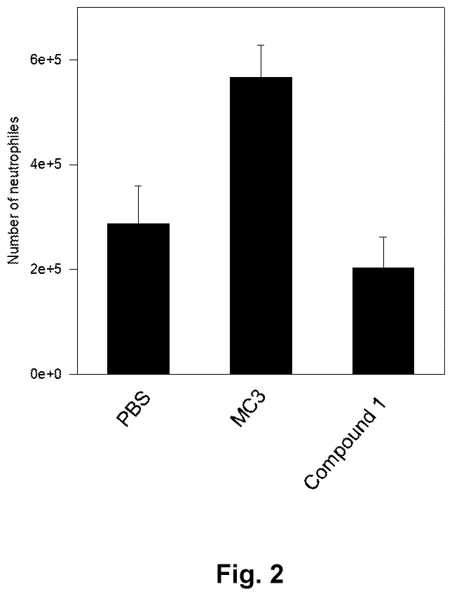

The expression of eGFP in rat lung confirmed the functionality of the applied LNP formulation in inducing the transcription of the cargo gene in vivo. The count of the eGFP molecules allows to assess efficacy relative to the MC3-based comparator formulation (see FIG. 1) with the LNP formulation comprising Compound 1. The measurement of chemokine release (e.g., neutrophil release) in the BAL assessed the inflammatory effect of each of the LNP formulations. The determination of the increase of chemokine release relative to the vehicle effect was used to compare LNP formulations with regard to the induction of an adverse inflammatory response in the treated tissue (see FIG. 2).

### Example 61. In Vivo Intracardiac Administration of LNP Formulations to Rat

Wistar rats were anaesthetized with Isoflurane and connected to a rat ventilator using a nose cone. The rats were ventilated with air ˜1100 ml/min and oxygen ˜100 ml/min (˜60) strokes/minute, (˜4 μl tidal volume). Core temperature was maintained at 37.5±1° C. by a heated operating table and a heating lamp controlled by a rectal thermometer.

The stomach and chest area was shaved and surgically scrubbed. Scissors was used to perform a left thoracotomy at the fifth intercostal space ˜2 to 3 mm to the left of the sternum. Marcain 5 ml/kg was given s.c as local analgesia at the site of thoracotomy. A rib spreader was used to keep the incision open. The pericardium was opened and a ligature with a 6-0 suture (Prolene) was placed to make it possible to lift the heart and for marking site of injections. The formulation was injected into the myocardium. The suture was tied with a loose knot and left in place, the chest was closed by suturing and the rat was kept heated and on ventilation until it regained consciousness. Temgesic 10 ml/kg was given s.c for long term analgesia before the rat was brought back to its cage.

For each formulation (Formulations 2 and 8 from Example 58), the rats were injected three times into the myocardium with a syringe (Myjector U-100 Insulin, 0.33 mm*12 mm) and with a compound volume of 20 μl per injection. The mRNA concentration of the formulation was 0.05 mg/mL and thus a total dose of 3 μg mRNA was given to each animal. N=3 animals per group. 24 h post dosing, the rats were anaesthetized with Isoflurane and when in a surgical plane of anesthesia heart puncture was performed to collect blood samples and to drain the heart from blood. Heart (right ventricle, divided into five 0.5-1 g sections) and liver (0.5-1 g of the right lobe) were harvested for protein quantification. All pieces of tissue were weighed before placed in Precellys tubes, snap frozen in liquid nitrogen and stored in −80° C. freezer until analysis. Blood was collected in EDTA tubes, placed on ice and the plasma was prepared within 30 minutes of sampling by centrifugation (3.000 RCF for 10 minutes at 4° C.). The plasma was separated into one 50 μl aliquot (Haptoglobin), and one 50 μl aliquot (Cytokines) and stored in −80° C. freezer until analysis.

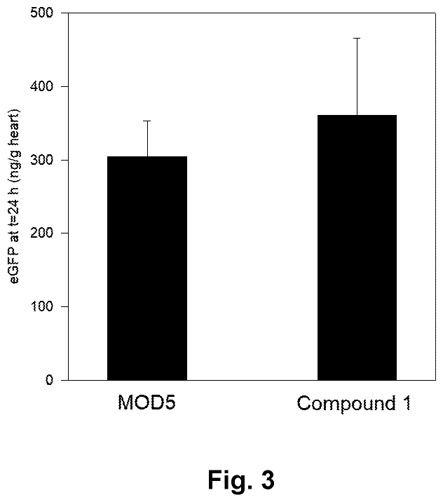

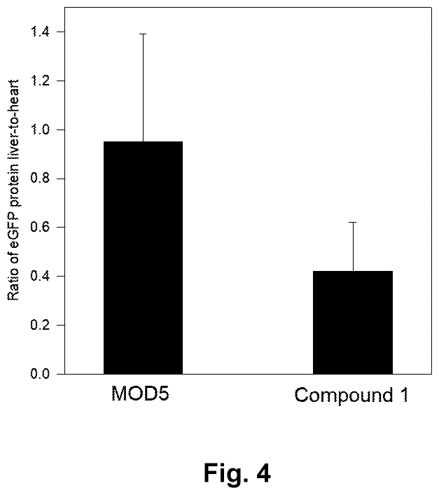

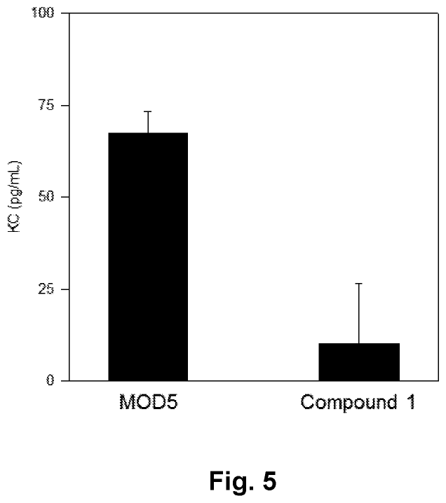

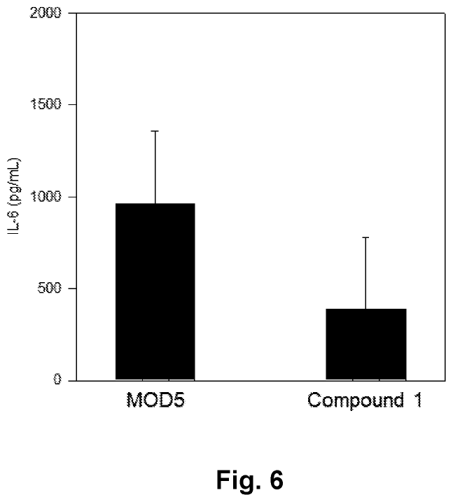

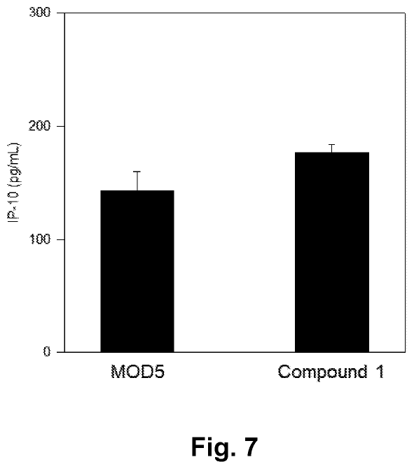

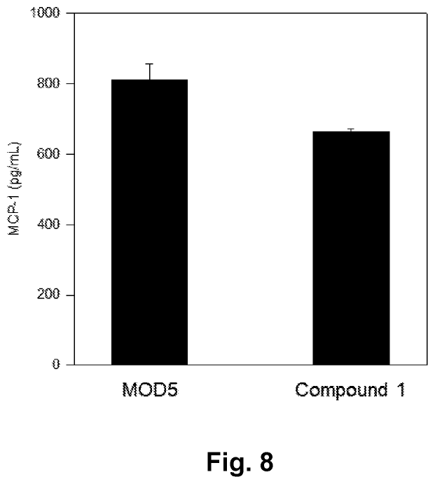

EGFP quantification in tissue samples was done by an EGFP ELISA (see FIGS. 3 and 4) and blood samples were used for analysis of Haptoglobin and Cytokines; IL-6, MCP-1, IP-10, KC (see FIGS. 5, 6, 7 and 8).

### Example 62. In Vivo Intravenous and Intramuscular Administration of LNP Formulations to Mouse

Female BALB/c mice were purchased (SPF (Beijing) Laboratory Animal Technology Co. Ltd.) and on arrival were caged in groups of 4 on corncob bedding, with normal diet and were provided tap drinking water ad libitum that was purified and autoclaved before being offered to the animal. The environment was maintained at a target temperature of 22±° C. and relative humidity of 40-80%, with a 12-hour light/dark cycle. Animals were acclimatised to the housing conditions for at least 7 days prior to any experimental procedures and were approximately 6-8 weeks of age at the start of dosing

Animals were assigned to respective groups such that the mean body weights for each treatment groups will be equal. N=4 for each treatment group.

Formulations 1, 3, 4, 5, and 6 from Example 58 were each dosed intravenously: Each mouse was removed from its cage and restrained, and then dosed with a slow IV bolus of a formulation in the lateral tail vein at a dose volume of 0.3 mg/kg.

Formulations 1, 5 and 9 from Example 58 were each dosed intramuscularly: Each mouse was removed from the cage and restrained, and then dosed in the caudal thigh area by slowly injecting a formulation into the muscle with a dose volume of 50 uL. All animals were checked for general condition post dosing to ensure the animals showed no ill signs following the treatment.

Blood samples were collected 6 hours after dose by the orbital plexus and 24 hours after dose at termination by cardiac puncture. The whole blood was collected into EDTA tubes and centrifuged for 10 minutes at 4000 rpm. The plasma was then analysed for cytokine, CRP and haptoglobin.

All animals were sacrificed 24 hours after dosing and flowing blood sampling the liver for the animals dosed by the IV route and the liver and muscle at the dose site for the IM dosed animals were harvested for eGFP analysis by ELISA.

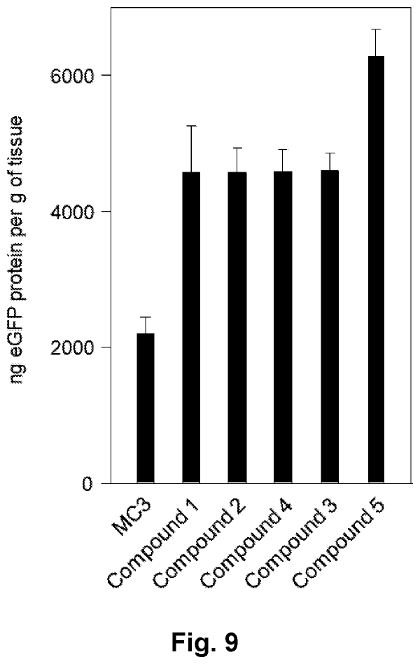

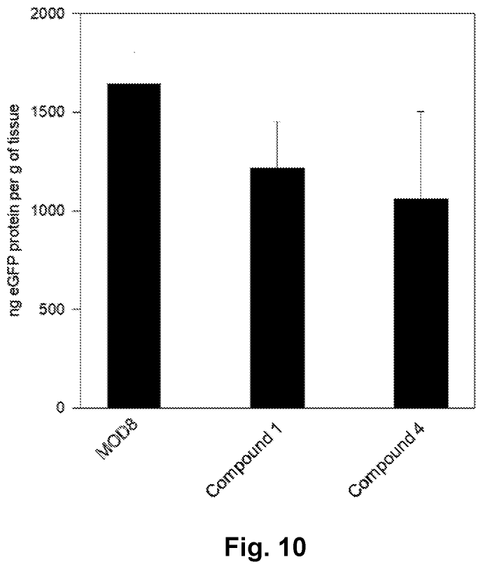

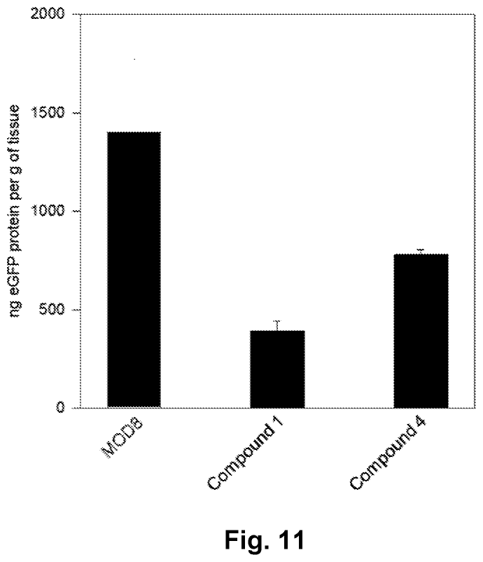

FIG. 9 shows the resulting eGFP expression in the liver at 24 hours after intravenous administration of LNP formulations. FIG. 10 shows the resulting eGFP expression in the muscle at 24 hours after intramuscular administration of LNP formulations. FIG. 11 shows the resulting eGFP expression in the liver at 24 hours after intramuscular administration of LNP formulations.

Additional LNP formulations were prepared according to the procedures described in Example 46. These formulations were tested using the same protocol as described herein. The results of eGFP expression in the liver at 24 hours after intravenous administration of the formulations were summarized in Tables 3, 4, 5 and 6 below.

TABLE 3

| | | | | | | | Liver eGFP | |
|---|---|---|---|---|---|---|---|---|
| Formulation | | N/P | Polymer | | <d>_{Z} | <d>_{N} | protein (ng/g | Std |
| No. | Ionizable lipid | ratio | conjugated lipid | % EE | (nm) | (nm) | tissue) | Error |
| 10 | Compound 6 | 3:1 | DMPE-PEG2000 | 87 | 77 | 59 | 3621.3 | 563.3 |
| 11 | Compound 8 | 3:1 | DMPE-PEG2000 | 95 | 76 | 61 | 6124.2 | 351.5 |
| 12 | Compound 7 | 3:1 | DMPE-PEG2000 | 94 | 77 | 54 | 6526.4 | 992.9 |
| 13 | Compound 9 | 3:1 | DMPE-PEG2000 | 93 | 81 | 61 | 3160.9 | 418.7 |
| 14 | Compound 13 | 3:1 | DMPE-PEG2000 | 93 | 66 | 49 | 8798.8 | 1737.7 |
| 15 | Compound 14 | 3:1 | DMPE-PEG2000 | 95 | 77 | 61 | 8056.3 | 842.1 |
| 16 | MC3 | 3:1 | DMPE-PEG2000 | 98 | 79 | 63 | 3091.5 | 350.2 |

TABLE 4

| | | | | | | | Liver eGFP | |
|---|---|---|---|---|---|---|---|---|
| Formulation | | N/P | Polymerconjugated | | <d>_{Z} | <d>_{N} | protein (ng/g | Std |
| No. | Ionizable lipid | ratio | lipid | % EE | (nm) | (nm) | tissue) | Error |
| 17 | Compound 24 | 3:1 | DMPE-PEG2000 | 94 | 71 | 52 | 748.9 | 162 |
| 18 | Compound 26 | 3:1 | DMPE-PEG2000 | 94 | 75 | 50 | 824.8 | 136 |
| 19 | Compound 25 | 3:1 | DMPE-PEG2000 | 94 | 69 | 50 | 15961.7 | 1990.1 |
| 20 | Compound 29 | 3:1 | DMPE-PEG2000 | 90 | 71 | 51 | 1288.4 | 158.7 |
| 21 | Compound 32 | 3:1 | DMPE-PEG2000 | 94 | 72 | 56 | 16518.2 | 2195 |
| 22 | Compound 33 | 3:1 | DMPE-PEG2000 | 97 | 67 | 53 | 1056.2 | 343.6 |
| 23 | Compound 19 | 3:1 | DMPE-PEG2000 | 92 | 75 | 57 | 9938.1 | 1036.3 |
| 24 | Compound 20 | 3:1 | DMPE-PEG2000 | 97 | 67 | 53 | 9757.7 | 752.7 |
| 25 | Compound 28 | 3:1 | DMPE-PEG2000 | 97 | 129 | 80 | 9557.1 | 346.5 |
| 26 | Compound 27 | 3:1 | DMPE-PEG2000 | 97 | 98 | 55 | 4982.6 | 764.2 |
| 27 | Compound 23 | 3:1 | DMPE-PEG2000 | 96 | 62 | 45 | 2318.8 | 472 |
| 28 | Compound 31 | 3:1 | DMPE-PEG2000 | 94 | 62 | 47 | 1319.3 | 440.5 |
| 29 | Compound 34 | 3:1 | DMPE-PEG2000 | 96 | 68 | 51 | 11015.4 | 876.7 |
| 30 | Compound 15 | 3:1 | DMPE-PEG2000 | 97 | 89 | 72 | 829.4 | 163.9 |
| 31 | Compound 16 | 3:1 | DMPE-PEG2000 | 91 | 78 | 61 | 1393.4 | 172.2 |
| 32 | Compound 17 | 3:1 | DMPE-PEG2000 | 93 | 96 | 77 | 6831.3 | 986.5 |
| 45 | Compound 45 | 3:1 | DMPE-PEG2000 | 88 | 75 | 60 | 9082.2 | 1227.3 |
| 33 | Compound 18 | 3:1 | DMPE-PEG2000 | 97 | 86 | 67 | 6817.5 | 496.9 |
| 34 | MC3 | 3:1 | DMPE-PEG2000 | 98 | 88 | 71 | 916.4 | 311 |

TABLE 5

| | | | | | | | Liver eGFP | |
|---|---|---|---|---|---|---|---|---|
| Formulation | | N/P | Polymer | | <d>_{Z} | <d>_{N} | protein (ng/g | Std |
| No. | Ionizable lipid | ratio | conjugated lipid | % EE | (nm) | (nm) | tissue) | Error |
| 35 | Compound 38 | 3:1 | DMPE-PEG2000 | 97 | 76 | 61 | 9730.7 | 1170.8 |
| 36 | Compound 22 | 3:1 | DMPE-PEG2000 | 95 | 90 | 63 | 4570.6 | 770.4 |
| 37 | Compound 42 | 3:1 | DMPE-PEG2000 | 94 | 84 | 66 | 1868.2 | 230.8 |
| 38 | Compound 44 | 3:1 | DMPE-PEG2000 | 97 | 80 | 65 | 6582.0 | 298.6 |
| 39 | Compound 37 | 3:1 | DMPE-PEG2000 | 93 | 60 | 47 | 1225.5 | 113.9 |
| 40 | Compound 41 | 3:1 | DMPE-PEG2000 | 98 | 69 | 54 | 7390.2 | 1155.4 |
| 41 | Compound 36 | 3:1 | DMPE-PEG2000 | 96 | 73 | 57 | 6133.5 | 587.8 |
| 42 | Compound 40 | 3:1 | DMPE-PEG2000 | 97 | 70 | 53 | 3658.4 | 450.2 |
| 43 | Compound 21 | 3:1 | DMPE-PEG2000 | 97 | 72 | 57 | 4103.6 | 397.8 |
| 44 | Compound 35 | 3:1 | DMPE-PEG2000 | 95 | 77 | 59 | 5974.6 | 686.1 |
| 45 | Compound 39 | 3:1 | DMPE-PEG2000 | 97 | 69 | 55 | 3383.8 | 291.0 |
| 46 | Compound 43 | 3:1 | DMPE-PEG2000 | 95 | 79 | 62 | 2590.9 | 609.2 |
| 47 | MC3 | 3:1 | DMPE-PEG2000 | 98 | 82 | 66 | 1114.4 | 185.2 |

TABLE 6

| | | | | | | | Liver eGFP | |
|---|---|---|---|---|---|---|---|---|
| Formulation | Ionizable | N/P | Polymer | | <d>_{Z} | <d>_{N} | protein (ng/g | Std |
| No. | lipid | ratio | conjugated lipid | % EE | (nm) | (nm) | tissue) | Error |
| 48 | Compound 46 | 3:1 | DMPE-PEG2000 | 93 | 73 | 54 | 15932.1 | 1203.3 |
| 49 | Compound 49 | 3:1 | DMPE-PEG2000 | 94 | 72 | 55 | 3934.6 | 587.3 |
| 50 | Compound 56 | 3:1 | DMPE-PEG2000 | 96 | 73 | 58 | 10270.4 | 1226.5 |
| 51 | Compound 55 | 3:1 | DMPE-PEG2000 | 93 | 77 | 62 | 18695.5 | 1269.5 |
| 52 | Compound 54 | 3:1 | DMPE-PEG2000 | 95 | 81 | 62 | 10704.5 | 530.2 |
| 53 | Compound 48 | 3:1 | DMPE-PEG2000 | 90 | 80 | 60 | 14749.9 | 802.5 |
| 54 | Compound 47 | 3:1 | DMPE-PEG2000 | 89 | 82 | 62 | 13883.6 | 1395.7 |
| 55 | Compound 50 | 3:1 | DMPE-PEG2000 | 97 | 65 | 51 | 7410.4 | 1062.9 |
| 56 | Compound 53 | 3:1 | DMPE-PEG2000 | 96 | 65 | 50 | 6947.2 | 612.9 |
| 57 | Compound 51 | 3:1 | DMPE-PEG2000 | 95 | 67 | 53 | 3473.8 | 362.9 |
| 58 | Compound 52 | 3:1 | DMPE-PEG2000 | 95 | 71 | 55 | 4701.7 | 1025.8 |
| 59 | MC3 | 3:1 | DMPE-PEG2000 | 98 | 74 | 61 | 3802.0 | 319.3 |

1. A compound of Formula (I): 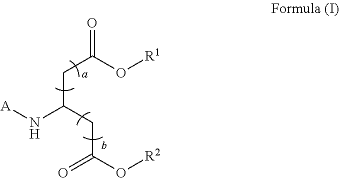
or a pharmaceutically acceptable salt thereof, wherein
A is 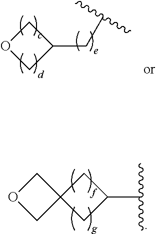
a and b are each independently 6, 7 or 8;
c, d, f and g are each independently 1 or 2;
e is 0, 1 or 2;
R¹ and R² are each independently 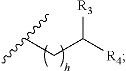
h is 0, 1, 2 or 3;
R³ and R⁴ are each independently —(CH₂)ᵢCH₃; and
i is 3, 4, 5, 6 or 7.

2. The compound of claim 1, wherein e is 0 or 1.

3. The compound of claim 1, wherein A is selected from

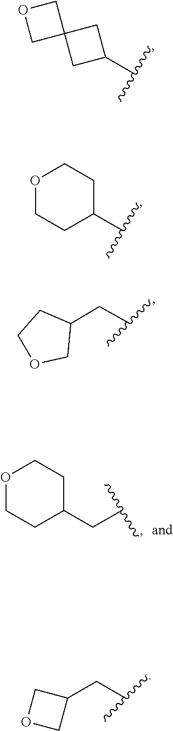 [SMILES: *C1CC2(COC2)C1 ; *C1CCOCC1 ; *CC1CCOC1 ; *CC1CCOCC1 ; *CC1COC1]

4. The compound of any one of the preceding claims, wherein a is 7.

5. The compound of any one of the preceding claims, wherein b is 7.

6. The compound of any one of the preceding claims, wherein h is 2.

7. The compound of any one of the preceding claims, wherein R³ and R⁴ are each —(CH₂)₄CH₃.

8. The compound of claim 1, wherein the compound is of Formula (II): 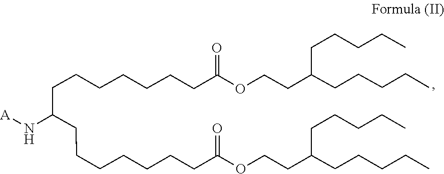
or a pharmaceutically acceptable salt thereof.

9-51. (canceled)

52. The compound of claim 1, or a pharmaceutically acceptable salt thereof, wherein the compound is selected from:
bis(3-pentyloctyl) 9-((2-oxaspiro[3.3]heptan-6-yl)amino)heptadecanedioate;
bis(3-pentyloctyl) 9-((tetrahydro-2H-pyran-4-yl)amino)heptadecanedioate;
bis(3-pentyloctyl) 9-(((tetrahydrofuran-3-yl)methyl)amino)heptadecanedioate;
bis(3-pentyloctyl) 9-(((tetrahydro-2H-pyran-4-yl)methyl)amino)heptadecanedioate; and
bis(3-pentyloctyl) 9-((oxetan-3-ylmethyl)amino)heptadecanedioate.

53. (canceled)

54. The compound of claim 1, wherein the compound is bis(3-pentyloctyl) 9-((2-oxaspiro[3.3]heptan-6-yl)amino)heptadecanedioate, or a pharmaceutically acceptable salt thereof.

55. A lipid nanoparticle comprising the compound of claim 1, or a pharmaceutically acceptable salt thereof.

56. The lipid nanoparticle of claim 55, wherein the lipid nanoparticle further comprises at least one neutral lipid, at least one sterol and at least one polymer-conjugated lipid.

57. The lipid nanoparticle of claim 56, wherein the neutral lipid is selected from distearoyl phosphatidylcholine (DSPC), dioleoyl phosphatidylethanolamine (DOPE), dipalmitoyl phosphatidylcholine (DPPC), dimyristoyl phosphatidylcholine (DMPC) or combinations thereof.

58. The lipid nanoparticle of claim 56, wherein the sterol is cholesterol.

59. The lipid nanoparticle of claim 56, wherein the polymer-conjugated lipid is selected from DMPE-PEG2000, DPPE-PEG2000, DMG-PEG2000, DPG-PEG2000, PEG2000-c-DOMG, PEG-C-DOPG or combinations thereof.

60. The lipid nanoparticle of claim 55, wherein the lipid nanoparticle further comprises distearoyl phosphatidylcholine (DSPC), cholesterol, and DMPE-PEG2000.

61. The lipid nanoparticle of claim 55, further comprising a nucleic acid segment.

62. The lipid nanoparticle of claim 61, wherein the nucleic acid segment is an RNA.

63. The lipid nanoparticle of claim 61, wherein the nucleic acid segment is a modified mRNA.

64. A pharmaceutical composition comprising a plurality of the lipid nanoparticles of claim 61.

65. A method of treating a disease or disorder in a subject comprising administering to the subject the pharmaceutical composition of claim 64, wherein the pharmaceutical composition comprises a therapeutically effective amount of the nucleic acid segment.

66-67. (canceled)

68. A lipid nanoparticle comprising the compound of claim 54, or a pharmaceutically acceptable salt thereof.

69. The lipid nanoparticle of claim 68, further comprising a nucleic acid segment.

70. A pharmaceutical composition comprising a plurality of the lipid nanoparticles of claim 69.
